(19) United States
(12) Patent Application Publication
(10) Pub. No.: US 2022/0267400 A1
(43) Pub. Date: Aug. 25, 2022

(54) IL-2 CYTOKINE PRODRUGS COMPRISING A CLEAVABLE LINKER

(71) Applicant: Trutino Biosciences Inc, San Diego, CA (US)

(72) Inventors: Phillip S. KIM, San Diego, CA (US); Emma LANGLEY, San Diego, CA (US); Hsieng LU, Thousand Oaks, CA (US); Xinjun LIU, San Diego, CA (US); Tani Ann LEE, San Diego, CA (US); Brian GROT, San Diego, CA (US)

(21) Appl. No.: 17/629,744

(22) PCT Filed: Jul. 24, 2020
(86) PCT No.: PCT/US2020/043616

§ 371 Date: Jan. 24, 2022

Related U.S. Application Data
(60) Provisional application No. 62/878,704, filed on Jul. 25, 2019.

(51) Int. Cl. C07K 14/55; A61K 47/68; A61P 37/04; C12N 15/62; A61K 47/65; A61P 35/00; C07K 16/30

(52) U.S. Cl. CPC C07K 14/55; A61K 47/68; A61P 37/04; C12N 15/62; A61K 47/65; A61P 35/00; C07K 16/30

(57) ABSTRACT

This disclosure relates to protease-cleavable IL-2 cytokine prodrugs. In some embodiments, the prodrugs comprise a pharmacokinetic modulator.

### CROSS REFERENCE

This application claims the benefit of priority to U.S. Provisional Patent Application No. 62/878,704, filed Jul. 25, 2019, which is incorporated herein by reference for all purposes.

### INCORPORATION BY REFERENCE OF SEQUENCE LISTING

The present application is being filed along with a Sequence Listing in electronic format. The Sequence Listing is provided as a file entitled 2020-07-22_54231-701_601_Sequence_ST25.txt, created Jul. 22, 2020, which is 199 kilobytes in size. The information in the electronic format of the Sequence Listing is incorporated by reference in its entirety.

### INTRODUCTION

This disclosure relates to the field of cytokine therapeutics, particularly cytokine prodrugs comprising a cleavable linker.

Cytokines, such as IL-2, are powerful immune growth factors that play a significant role in sustaining an effective immune cell response. IL-2 has been reported to induce complete and durable regressions in cancer patients but immune related adverse effects have reduced its therapeutic potential. In some cases, however, systemic IL-2 administration can activate immune cells throughout the body. Systemic activation can lead to systemic toxicity and indiscriminate activation of immune cells, including immune cells that respond to a variety of epitopes, antigens, and stimuli. The therapeutic potential of IL-2 therapy can be impacted by these severe toxicities.

IL-2 therapies can also suffer from a short serum half-life, sometimes on the order of several minutes. Thus, the high doses of IL-2 that can be necessary to achieve an optimal immune-modulatory effect can contribute to severe toxicities.

As a result, cytokine therapeutics that overcome the hurdles of systemic or untargeted function, severe toxicity, and poor pharmacokinetics, are needed. The present disclosure aims to meet one or more of these needs, provide other benefits, or at least provide the public with a useful choice.

In some aspects, protease-activated pro-cytokines (also referred to as cytokine prodrugs) are provided, which can be administered to a subject in an inactive form. The inactive form can include a cytokine polypeptide sequence, a protease-cleavable polypeptide sequence, and an inhibitory polypeptide sequence capable of blocking an activity of the cytokine polypeptide sequence. Such prodrugs can become activated when the protease-cleavable polypeptide sequence is cleaved by a protease. Cleaving the protease-cleavable polypeptide can allow the inhibitory polypeptide sequence to dissociate from the cytokine polypeptide sequence.

Many tumors and tumor microenvironments exhibit aberrant expression of proteases. The present disclosure provides cytokine prodrugs that are activatable through proteolytic cleavage, such that they become active when they come in contact with proteases in a tumor or tumor microenvironment. In some cases, this can lead to an increase in active cytokines in and around the tumor or tumor microenvironment relative to the rest of a subject's body or healthy tissue. One exemplary advantage that can result is the formation of cytokine gradients. Such a gradient can form when a cytokine prodrug is administered and selectively or preferentially becomes activated in the tumor or tumor microenvironment and subsequently diffuses out of these areas to the rest of the body. These gradients can increase the trafficking of immune cells to the tumor and tumor microenvironment. Immune cells that traffic to the tumor can infiltrate the tumor. Infiltrating immune cells can mount an immune response against the cancer. Infiltrating immune cells can also secrete their own chemokines and cytokines. The cytokines can have either or both of autocrine and paracrine effects within the tumor and tumor microenvironment. In some cases, the immune cells include T cells, such as T effector cells or cytotoxic T cells, or NK cells.

Also described herein are methods of treatment and methods of administrating the cytokine prodrugs described herein. Such administration can be systemic or local. In some embodiments, a cytokine prodrug described herein is administered systemically or locally to treat a cancer.

A further example of local administration is administration of a cytokine prodrug, such as an IL-2 cytokine prodrug, to boost T regulatory cells. In some cases, the local administration of an IL-2 cytokine prodrug is to an area of inflammation. Such a method can be used to treat chronic autoimmune and/or inflammatory diseases.

### SUMMARY

The following embodiments are encompassed.

Embodiment 1 is a protease-activated pro-cytokine comprising:

a cytokine polypeptide sequence;
a inhibitory polypeptide sequence capable of blocking an activity of the cytokine polypeptide sequence; and
a linker between the cytokine polypeptide sequence and the inhibitory polypeptide sequence, the linker comprising a protease-cleavable polypeptide sequence;
wherein:
i) the protease-cleavable polypeptide sequence is a protease-cleavable polypeptide sequence comprising any one of SEQ ID NOs: 80-94 or 201-242, or a variant having one or two mismatches relative to the sequence of any one of SEQ ID NOs: 80-90 or 201-242.

Embodiment 2 is the protease-activated pro-cytokine of the immediately preceding embodiment, further comprising a pharmacokinetic modulator. Embodiment 3 is the protease-activated pro-cytokine of the immediately preceding embodiment, wherein the pharmacokinetic modulator comprises an immunoglobulin constant domain. Embodiment 4 is the protease-activated pro-cytokine of embodiment 2, wherein the pharmacokinetic modulator comprises an immunoglobulin Fc region, optionally wherein the Fc region is a knob-into-hole heterodimeric Fc region. Embodiment 5 is the protease-activated pro-cytokine of the immediately preceding embodiment, wherein the immunoglobulin Fc region is a human immunoglobulin Fc region. Embodiment 6 is the protease-activated pro-cytokine of any one of embodiments 4-5, wherein the immunoglobulin Fc region is an IgG Fc region. Embodiment 7 is the protease-activated pro-cytokine of the immediately preceding embodiment, wherein the IgG Fc region is an IgG1, IgG2, IgG3, or IgG4 Fc region.

Embodiment 8 is the protease-activated pro-cytokine of embodiment 2, wherein the pharmacokinetic modulator comprises an albumin. Embodiment 9 is the protease-activated pro-cytokine of the immediately preceding embodiment, wherein the albumin is a serum albumin. Embodiment 10 is the protease-activated pro-cytokine of any one of embodiments 8-9, wherein the albumin is a human albumin. Embodiment 11 is the protease-activated pro-cytokine of embodiment 2, wherein the pharmacokinetic modulator comprises PEG. Embodiment 12 is the protease-activated pro-cytokine of embodiment 2, wherein the pharmacokinetic modulator comprises XTEN. Embodiment 13 is the protease-activated pro-cytokine of embodiment 2, wherein the pharmacokinetic modulator comprises CTP. Embodiment 14 is the protease-activated pro-cytokine of any one of embodiments 2-13, wherein the protease-cleavable polypeptide sequence is between the cytokine polypeptide sequence and the pharmacokinetic modulator. Embodiment 15 is the protease-activated pro-cytokine of any one of embodiments 2-13, wherein the pharmacokinetic modulator is between the cytokine polypeptide sequence and the protease-cleavable polypeptide sequence.

Embodiment 16 is the protease-activated pro-cytokine of any one of the preceding embodiments, comprising a plurality of protease-cleavable polypeptide sequences. Embodiment 17 is the protease-activated pro-cytokine of the immediately preceding embodiment, wherein the cytokine polypeptide sequence is flanked by protease cleavable polypeptide sequences. Embodiment 18 is the protease-activated pro-cytokine of the immediately preceding embodiment, having the structure PM-CL-CY-CL-IN (from N- to C-terminus or from C- to N-terminus), where PM is the pharmacokinetic modulator, each CL independently is a protease-cleavable polypeptide sequence, CY is the cytokine polypeptide sequence, and IN is the inhibitory polypeptide sequence.

Embodiment 19 is the protease-activated pro-cytokine of any one of the preceding embodiments, wherein the cytokine polypeptide sequence comprises a modification to prevent disulfide bond formation, and optionally otherwise comprises wild-type sequence.

Embodiment 20 is the protease-activated pro-cytokine of any one of the preceding embodiments, wherein the cytokine polypeptide sequence has at least 80, 85, 90, 95, 97, 98, or 99 percent identity to the sequence of a wild-type cytokine polypeptide sequence or to a cytokine polypeptide sequence in Table 1. Embodiment 21 is the protease-activated pro-cytokine of the immediately preceding embodiment, wherein the cytokine polypeptide sequence is a wild-type cytokine polypeptide sequence. Embodiment 22 is the protease-activated pro-cytokine of any one of the preceding embodiments, wherein the cytokine is a monomeric cytokine or a dimeric cytokine, wherein the monomers are associated noncovalently or covalently directly or indirectly via a linker. Embodiment 23 is the protease-activated pro-cytokine of any one of the preceding embodiments, wherein the inhibitory polypeptide sequence comprises a cytokine-binding domain. Embodiment 24 is the protease-activated pro-cytokine of the immediately preceding embodiment, wherein the cytokine-binding domain is a cytokine-binding domain of a cytokine receptor or a cytokine-binding domain of a fibronectin. Embodiment 25 is the protease-activated pro-cytokine of the immediately preceding embodiment, wherein the cytokine-binding domain comprises the sequence of any one of SEQ ID NOs: 10-29 or 40-51. Embodiment 26 is the protease-activated pro-cytokine of embodiment 24, wherein the cytokine-binding domain is an immunoglobulin cytokine-binding domain. Embodiment 27 is the protease-activated pro-cytokine of the immediately preceding embodiment, wherein the immunoglobulin cytokine-binding domain comprises a light chain variable domain and a heavy chain variable domain that bind the cytokine. Embodiment 28 is the protease-activated pro-cytokine of any one of embodiments 26-27, wherein the immunoglobulin cytokine-binding domain is an scFv or Fab.

Embodiment 29 is the protease-activated pro-cytokine of any one of the preceding embodiments, wherein the protease-cleavable polypeptide sequence is cleavable by at least one of a metalloprotease, a serine protease, a cysteine protease, an aspartate protease, a threonine protease, a glutamate protease, a gelatinase, an asparagine peptide lyase, a cathepsin, a kallikrein, a plasmin, a collagenase, a hK1, a hK10, a hK15, a stromelysin, a Factor Xa, a chymotrypsin-like protease, a trypsin-like protease, a elastase-like protease, a subtilisin-like protease, an actinidain, a bromelain, a calpain, a caspase, a Mir 1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, an ADAM 10, an ADAM17, an ADAM 12, an urokinase plasminogen activator (uPA), an enterokinase, a prostate-specific target (PSA, hK3), an interleukin-1b converting enzyme, a thrombin, a FAP (FAP-a), a dipeptidyl peptidase, or dipeptidyl peptidase IV (DPPIV/CD26), a type II transmembrane serine protease (TTSP), a neutrophil elastase, a proteinase 3, a mast cell chymase, a mast cell tryptase, or a dipeptidyl peptidase.

Embodiment 30 is the protease-activated pro-cytokine of any one of the preceding embodiments, wherein the protease-cleavable polypeptide sequence comprises the sequence of any one of SEQ ID NOs: 201-242, or a variant having one or two mismatches relative to the sequence of any one of SEQ ID NOs: 201-242.

Embodiment 31 is the protease-activated pro-cytokine of any one of the preceding embodiments, wherein the protease-cleavable polypeptide sequence is cleavable by a matrix metalloprotease. Embodiment 32 is the protease-activated pro-cytokine of any one of the preceding embodiments, wherein the protease-cleavable polypeptide sequence is cleavable by MMP-1. Embodiment 33 is the protease-activated pro-cytokine of any one of the preceding embodiments, wherein the protease-cleavable polypeptide sequence is cleavable by MMP-2. Embodiment 34 is the protease-activated pro-cytokine of any one of the preceding embodiments, wherein the protease-cleavable polypeptide sequence is cleavable by MMP-3. Embodiment 35 is the protease-activated pro-cytokine of any one of the preceding embodiments, wherein the protease-cleavable polypeptide sequence is cleavable by MMP-7. Embodiment 36 is the protease-activated pro-cytokine of any one of the preceding embodiments, wherein the protease-cleavable polypeptide sequence is cleavable by MMP-8. Embodiment 37 is the protease-activated pro-cytokine of any one of the preceding embodiments, wherein the protease-cleavable polypeptide sequence is cleavable by MMP-9.

Embodiment 38 is the protease-activated pro-cytokine of any one of the preceding embodiments, wherein the protease-cleavable polypeptide sequence is cleavable by MMP-12. Embodiment 39 is the protease-activated pro-cytokine of any one of the preceding embodiments, wherein the protease-cleavable polypeptide sequence is cleavable by MMP-13. Embodiment 40 is the protease-activated pro-cytokine of any one of the preceding embodiments, wherein the protease-cleavable polypeptide sequence is cleavable by MMP-14. Embodiment 41 is the protease-activated pro-cytokine of any one of the preceding embodiments, wherein the protease-cleavable polypeptide sequence is cleavable by more than one MMP. Embodiment 42 is the protease-activated pro-cytokine of any one of the preceding embodiments, wherein the protease-cleavable polypeptide sequence is cleavable by two, three, four, five, six, or seven of MMP-2, MMP-7, MMP-8, MMP-9, MMP-12, MMP-13, and MMP-14.

Embodiment 43 is the protease-activated pro-cytokine of any one of the preceding embodiments, wherein the protease-cleavable polypeptide sequence comprises the sequence of any one of SEQ ID NOs: 80-94 or a variant sequence having one or two mismatches relative to the sequence of any one of SEQ ID NOs: 80-90. Embodiment 44 is the protease-activated pro-cytokine of the immediately preceding embodiment, wherein the protease-cleavable polypeptide sequence comprises the sequence of SEQ ID NO: 80 or a variant sequence having one or two mismatches relative thereto. Embodiment 45 is the protease-activated pro-cytokine of any one of embodiments 1-43, wherein the protease-cleavable polypeptide sequence comprises the sequence of SEQ ID NO: 81 or a variant sequence having one or two mismatches relative thereto. Embodiment 46 is the protease-activated pro-cytokine of any one of embodiments 1-43, wherein the protease-cleavable polypeptide sequence comprises the sequence of SEQ ID NO: 82 or a variant sequence having one or two mismatches relative thereto. Embodiment 47 is the protease-activated pro-cytokine of any one of embodiments 1-43, wherein the protease-cleavable polypeptide sequence comprises the sequence of SEQ ID NO: 83 or a variant sequence having one or two mismatches relative thereto. Embodiment 48 is the protease-activated pro-cytokine of any one of embodiments 1-43, wherein the protease-cleavable polypeptide sequence comprises the sequence of SEQ ID NO: 84 or a variant sequence having one or two mismatches relative thereto. Embodiment 49 is the protease-activated pro-cytokine of any one of embodiments 1-43, wherein the protease-cleavable polypeptide sequence comprises the sequence of SEQ ID NO: 85 or a variant sequence having one or two mismatches relative thereto. Embodiment 50 is the protease-activated pro-cytokine of any one of embodiments 1-43, wherein the protease-cleavable polypeptide sequence comprises the sequence of SEQ ID NO: 86 or a variant sequence having one or two mismatches relative thereto. Embodiment 51 is the protease-activated pro-cytokine of any one of embodiments 1-43, wherein the protease-cleavable polypeptide sequence comprises the sequence of SEQ ID NO: 87 or a variant sequence having one or two mismatches relative thereto. Embodiment 52 is the protease-activated pro-cytokine of any one of embodiments 1-43, wherein the protease-cleavable polypeptide sequence comprises the sequence of SEQ ID NO: 88 or a variant sequence having one or two mismatches relative thereto. Embodiment 53 is the protease-activated pro-cytokine of any one of embodiments 1-43, wherein the protease-cleavable polypeptide sequence comprises the sequence of SEQ ID NO: 89 or a variant sequence having one or two mismatches relative thereto. Embodiment 54 is the protease-activated pro-cytokine of any one of embodiments 1-43, wherein the protease-cleavable polypeptide sequence comprises the sequence of SEQ ID NO: 90 or a variant sequence having one or two mismatches relative thereto. Embodiment 55 is the protease-activated pro-cytokine of any one of embodiments 1-43, wherein the protease-cleavable polypeptide sequence comprises the sequence of SEQ ID NO: 80-89 or 90. Embodiment 56 is the protease-activated pro-cytokine of any one of embodiments 1-43, wherein the protease-cleavable polypeptide sequence comprises the sequence of SEQ ID NO: 91. Embodiment 57 is the protease-activated pro-cytokine of any one of embodiments 1-43, wherein the protease-cleavable polypeptide sequence comprises the sequence of SEQ ID NO: 92. Embodiment 58 is the protease-activated pro-cytokine of any one of embodiments 1-43, wherein the protease-cleavable polypeptide sequence comprises the sequence of SEQ ID NO: 93. Embodiment 59 is the protease-activated pro-cytokine of any one of embodiments 1-43, wherein the protease-cleavable polypeptide sequence comprises the sequence of SEQ ID NO: 94. Embodiment 60 is the protease-activated pro-cytokine of any one of the preceding embodiments, wherein the cytokine polypeptide sequence is an IL-2 polypeptide sequence.

Embodiment 61 is the protease-activated pro-cytokine of the immediately preceding embodiment, wherein the IL-2 polypeptide sequence has at least 80, 85, 90, 95, 97, 98, or 99 percent identity to the sequence of any one of SEQ ID NOs: 1-4. Embodiment 62 is the protease-activated pro-cytokine of the immediately preceding embodiment, wherein the IL-2 polypeptide sequence comprises the sequence of any one of SEQ ID NOs: 1-4. Embodiment 63 is the protease-activated pro-cytokine of any one of embodiments 60-62, wherein the IL-2 polypeptide sequence is a human IL-2 polypeptide sequence. Embodiment 64 is the protease-activated pro-cytokine of the immediately preceding embodiment, wherein the IL-2 polypeptide sequence comprises the sequence of SEQ ID NO: 1. Embodiment 65 is the protease-activated pro-cytokine of any one of embodiment 62, wherein the IL-2 polypeptide sequence comprises the sequence of SEQ ID NO: 2.

Embodiment 66 is the protease-activated pro-cytokine of any one of embodiments 60-65, wherein the inhibitory polypeptide sequence comprises an IL-2 binding domain of an IL-2 receptor (IL-2R). Embodiment 67 is the protease-activated pro-cytokine of the immediately preceding embodiment, wherein the inhibitory polypeptide sequence comprises an amino acid sequence having at least 80, 85, 90, 95, 97, 98, or 99 percent identity to the sequence of any one of SEQ ID NOs: 10-29 or 40-51. Embodiment 68 is the protease-activated pro-cytokine of the immediately preceding embodiment, wherein the IL-2R is a human IL-2R. Embodiment 69 is the protease-activated pro-cytokine of any one of embodiments 60-65, wherein the inhibitory polypeptide sequence comprises an IL-2-binding immunoglobulin domain. Embodiment 70 is the protease-activated pro-cytokine of embodiment 69, wherein the IL-2-binding immunoglobulin domain is a human IL-2-binding immunoglobulin domain.

Embodiment 71 is the protease-activated pro-cytokine of the immediately preceding embodiment, wherein the IL-2-binding immunoglobulin domain comprises a VL region comprising hypervariable regions (HVRs) HVR-1, HVR-2, and HVR-3 having the sequences of SEQ ID NOs: 33, 34, and 35, respectively, and a VH region comprising HVR-1, HVR-2, and HVR-3 having the sequences of SEQ ID NOs: 36, 37, and 38, respectively; or the IL-2-binding immunoglobulin domain comprises a VL region comprising hypervariable regions (HVRs) HVR-1, HVR-2, and HVR-3 having the sequences of SEQ ID NOs: 250, 251, and 252, respectively, and a VH region comprising HVR-1, HVR-2, and HVR-3 having the sequences of SEQ ID NOs: 253, 254, and 255, respectively. Embodiment 72 is the protease-activated pro-cytokine of any one of embodiments 69-71, wherein the IL-2-binding immunoglobulin domain comprises a VL region comprising an amino acid sequence having at least 80, 85, 90, 95, 97, 98, or 99 percent identity to the sequence of SEQ ID NO: 32 and a VH region comprising an amino acid sequence having at least 80, 85, 90, 95, 97, 98, or 99 percent identity to the sequence of SEQ ID NO: 33; or the IL-2-binding immunoglobulin domain comprises a VL region comprising an amino acid sequence having at least 80, 85, 90, 95, 97, 98, or 99 percent identity to the sequence of SEQ ID NO: 249 and a VH region comprising an amino acid sequence having at least 80, 85, 90, 95, 97, 98, or 99 percent identity to the sequence of SEQ ID NO: 248. Embodiment 73 is the protease-activated pro-cytokine of the immediately preceding embodiment, wherein the IL-2-binding immunoglobulin domain comprises a VL region comprising the sequence of SEQ ID NO: 32 and a VH region comprising the sequence of SEQ ID NO: 33; or the IL-2-binding immunoglobulin domain comprises a VL region comprising the sequence of SEQ ID NO: 249 and a VH region comprising the sequence of SEQ ID NO: 248. Embodiment 74 is the protease-activated pro-cytokine of any one of embodiments 89-93, wherein the IL-2-binding immunoglobulin domain is an scFv. Embodiment 75 is the protease-activated pro-cytokine of the immediately preceding embodiment, wherein the IL-2-binding immunoglobulin domain comprises an amino acid sequence having at least 80, 85, 90, 95, 97, 98, or 99 percent identity to the sequence of SEQ ID NO: 30, 31, or 247. Embodiment 76 is the protease-activated pro-cytokine of the immediately preceding embodiment, wherein the IL-2-binding immunoglobulin domain comprises the sequence of SEQ ID NO: 30, 31, or 247.

Embodiment 77 is a pharmaceutical composition comprising the protease-activated pro-cytokine of any one of the preceding embodiments.

Embodiment 78 is the protease-activated pro-cytokine or pharmaceutical composition of any one of the preceding embodiments, for use in therapy.

Embodiment 79 is the protease-activated pro-cytokine or pharmaceutical composition of any one of the preceding embodiments, for use in treating a cancer.

Embodiment 80 is a method of treating a cancer, comprising administering the protease-activated pro-cytokine or pharmaceutical composition of any one of the preceding embodiments to a subject in need thereof.

Embodiment 81 is the use of the protease-activated pro-cytokine or pharmaceutical composition of any one of embodiments 1-77 for the manufacture of a medicament for treating cancer.

Embodiment 82 is a method of creating a cytokine gradient in a subject, comprising administering the protease-activated pro-cytokine or pharmaceutical composition of any one of embodiments 1-77 to a subject, wherein the subject comprises a site having an abnormally high level of a protease that cleaves the protease-cleavable polypeptide sequence, optionally wherein the site comprises a cancer.

Embodiment 83 is the protease-activated pro-cytokine or pharmaceutical composition of any one of embodiments 1-77, for use in a method of creating a cytokine gradient in a subject, comprising administering the protease-activated pro-cytokine or pharmaceutical composition to a subject, wherein the subject comprises a site having an abnormally high level of a protease that cleaves the protease-cleavable polypeptide sequence, optionally wherein the site comprises a cancer.

Embodiment 84 is the use of the protease-activated pro-cytokine or pharmaceutical composition of any one of embodiments 1-77 for the manufacture of a medicament for creating a cytokine gradient in a subject, comprising administering the protease-activated pro-cytokine or pharmaceutical composition to a subject, wherein the subject comprises a site having an abnormally high level of a protease that cleaves the protease-cleavable polypeptide sequence, optionally wherein the site comprises a cancer.

Embodiment 85 is the method, use, or protease-activated pro-cytokine for use of any one of embodiments 79-84, wherein the cancer is a solid tumor. Embodiment 86 is the method, use, or protease-activated pro-cytokine for use of the immediately preceding embodiment, wherein the solid tumor is metastatic and/or unresectable. Embodiment 87 is the method, use, or protease-activated pro-cytokine for use of any one of embodiments 79-86, wherein the cancer is a PD-L1-expressing cancer. Embodiment 88 is the method, use, or protease-activated pro-cytokine for use of any one of embodiments 79-87, wherein the cancer is a melanoma, a colorectal cancer, a breast cancer, a pancreatic cancer, a lung cancer, a prostate cancer, an ovarian cancer, a cervical cancer, a gastric or gastrointestinal cancer, a lymphoma, a colon or colorectal cancer, an endometrial cancer, a thyroid cancer, or a bladder cancer. Embodiment 89 is the method, use, or protease-activated pro-cytokine for use of any one of embodiments 79-88, wherein the cancer is a microsatellite instability-high cancer. Embodiment 90 is the method, use, or protease-activated pro-cytokine for use of any one of embodiments 79-89, wherein the cancer is mismatch repair deficient.

Embodiment 91 is a nucleic acid encoding the protease-activated pro-cytokine of any one of embodiments 1-76. Embodiment 92 is an expression vector comprising the nucleic acid of embodiment 91. Embodiment 93 is a host cell comprising the nucleic acid of embodiment 91 or the vector of embodiment 92.

Embodiment 94 is a method of producing a protease-activated pro-cytokine, comprising culturing the host cell of embodiment 93 under conditions wherein the protease-activated pro-cytokine is produced. Embodiment 95 is the method of the immediately preceding embodiment, further comprising isolating the protease-activated pro-cytokine.

Embodiment 96 is a method of boosting T regulatory cells and/or reducing inflammation or autoimmune activity, comprising administering the protease-activated pro-cytokine of any one of embodiments 1-77 to an area of interest in a subject, e.g., an area of inflammation in the subject.

Embodiment 97 is a method of treating an inflammatory or autoimmune disease or disorder in a subject, comprising administering the protease-activated pro-cytokine of any one of embodiments 1-77 to an area of interest in a subject, e.g., an area of inflammation or autoimmune activity in the subject.

### BRIEF DESCRIPTION OF THE DRAWINGS

FIG. 1A shows an illustration of an exemplary cytokine prodrug structure and an SDS-PAGE gel characterizing a purified cytokine prodrug (Construct B). Abbreviations: PM, pharmacokinetic modulator; HMW, high molecular weight.

FIG. 1B shows an illustration of an exemplary cytokine prodrug structure comprising human IL-2 and IL-2Ra sequences and an MMP-cleavable linker, and an SDS-PAGE gel and Western blot characterizing a purified cytokine prodrug (Construct E). Abbreviations: Hu, human; MMP, matrix metalloprotease; other abbreviations are as above.

FIG. 2A illustrates a cleavage reaction of a cytokine prodrug by a protease and shows Western blot evidence of cleavage of Construct A by MMP-9 at time points of 1, 2, and 4 hours and overnight. Each of the Western blots contains +MMP digestion lanes and −MMP mock-digestion lanes. Cleavage product was detectable at 1 hour, and the full-length cytokine prodrug was substantially undetectable at the overnight +MMP time point.

FIG. 2B illustrates a cleavage reaction of a cytokine prodrug comprising a pharmacokinetic modulator by a protease and shows Western blot evidence of cleavage of Construct B by MMP-9 at time points of 1, 4, and 20 hours. Each of the Western blots contains +MMP digestion lanes and −MMP mock-digestion lanes. Cleavage product was detectable at 1 hour, and the full-length cytokine prodrug gave only a faint band at the 20 hour +MMP time point.

FIG. 2C illustrates a cleavage reaction of a cytokine prodrug comprising a pharmacokinetic modulator by a protease and shows Western blot evidence of cleavage of Construct E by MMP-9 at time points of 1, 4, and 22 hours. Each of the Western blots contains +MMP9 digestion lanes and −MMP9 mock-digestion lanes. Cleavage product was detectable at 1 hour, and the full-length cytokine prodrug gave essentially no band at the 22 hour +MMP time point.

FIG. 3A shows results of a CTLL-2 proliferation assay with Construct A or cleavage products thereof. Construct A was cleaved by MMP-9 and the resulting products were incubated with CTLL-2 cells. The data shows that MMP-9 treated Construct A stimulates CTLL-2 cell proliferation in a dose dependent manner and exhibits 10-fold greater activity than untreated Construct A (EC50 comparison). EC50 values are shown in nM.

FIG. 3B shows results of a CTLL-2 proliferation assay with Construct B or cleavage products thereof. Construct B was cleaved by MMP-9 and the resulting products were incubated with CTLL-2 cells. For comparison, mIL2 was also incubated with CTLL-2 cells. The data show that MMP-9 treated Construct B stimulates CTLL-2 cell proliferation in a dose dependent manner. Uncleaved Construct B was minimally stimulatory. EC50 values are shown in nM.

FIG. 3C-FIG. 3J show HEK-Blue™ IL2 assay results. Cells were treated with various concentrations Construct E, uncleaved or cleaved with mMMP9 for 22 hours (FIG. 3C); human IL2 (FIG. 3D); Construct B, uncleaved or cleaved with mMMP9 for 19 hours; Construct J, Construct K, Construct F, Construct L, or Construct I, each uncleaved or cleaved with mMMP9 for 22 hours (FIGS. 3E-J, respectively); and the EC50 was determined based on OD630 as a readout of IL-2 stimulation.

FIG. 3K-FIG. 3L show results of a CTLL-2 proliferation assay with Construct M, Construct N, or cleavage products thereof. Cleavage was by MMP-2 for 2 hr and the resulting products were incubated with CTLL-2 cells. The data show that MMP-2 treated Construct M and Construct N stimulate CTLL-2 cell proliferation in a dose dependent manner. EC50 values are shown in nM.

FIG. 3M shows Coomassie-stained SDS-PAGE results comparing Construct E, Construct M, and Construct N. Construct M and Construct N showed decreased aggregation and greater stability and homogeneity.

FIG. 3N-FIG. 3P show results of a CTLL-2 proliferation assay with Construct O, Construct P, Construct Q, or cleavage products thereof. Cleavage was by MMP2 for 2 hr and the resulting products were incubated with CTLL-2 cells. The data show that MMP2 treated Construct O, Construct P, and Construct Q stimulate CTLL-2 cell proliferation in a dose dependent manner. EC50 values are shown in nM.

FIG. 4 illustrates a serum stability assay using Construct B and provides results thereof indicating that Construct B was stable when incubated with serum collected from control or tumor-bearing over a time course of 72 hours. Concentrations were measured by quantitative sandwich ELISA using an mIL2 capture antibody and mIL2Ra detection antibody.

FIG. 5 shows a study design, graphical results, and pharmacokinetic (PK) parameters for Construct B in mice. PK parameters were calculated using WinNonlin 7.0 (non-compartmental model).

FIG. 6A shows a study design and results for intratumoral dosing of Construct A in mice injected subcutaneously with MC38 cells at day −7 and then treated with Construct A, vehicle, or human IL-2 on each of days 0-4 and 7-11. Construct A substantially inhibited tumor growth. In contrast, human IL-2 adversely affected tumor control relative to vehicle. Necrosis attributable to tumor growth was observed in the control and human IL-2 groups.

FIG. 6B shows a study design in which mice treated as in FIG. 6A were re-challenged with 2×10⁶ MC38 cells at day 40. Tumor growth was rejected, indicating that the treatment resulted in a durable response including anti-tumor immune memory.

FIG. 7A shows a study design in mice injected subcutaneously with MC38 cells at day −10 where Construct B or vehicle was administered intravenously once per three days (Q3D) during a three week period (eight total administrations). Essentially no systemic toxicity was observed. Construct B-treated mice showed virtually no tumor growth after initiation of treatment, in contrast to vehicle-treated mice where tumor growth continued through day 21. Following day 21, several vehicle-treated mice were euthanized due to tumor volume exceeding 3000 mm³ and accordingly subsequent tumor volume data for vehicle-treated mice is not shown as it would be biased toward mice with smaller tumor volumes relative to the population average through day 21.

FIG. 7B shows body weight data for the same mice as in FIG. 7A. Mouse body weight was substantially constant during treatment with Construct B, consistent with lack of any apparent toxicity.

FIG. 8 shows immunohistochemistry results for tumor-infiltrating immune cells at day 21 for vehicle group tissues and at day 25 for Construct B treated tumors of the study described above for FIG. 7A. Significantly more immune cells of all tested types were observed in Construct B-treated mice compared to vehicle-treated mice. Additionally, the proportion of cells with markers consistent with a effector T cell phenotype was substantially greater than the proportion of CD4+Foxp3+ (regulatory T) cells. Statistical analysis was performed using unpaired t test by Prism 5.0 software. P value between groups was calculated, and the differences with p value <0.05 were considered statistically significant. *p<0.05, **p<0.01, ***p<0.001.

FIG. 9 shows quantification of MMP activity in the indicated tumor-bearing mouse models by fluorescence intensity over time following MMPSense 680™ injection.

FIG. 10A-FIG. 10D show tumor volume over time for mice treated with vehicle or Construct B as indicated in the indicated cancer models.

FIG. 11A-FIG. 11D show tumor volume over time (11A) and levels of the indicated enzymes (11B-D) for mice treated with vehicle or Construct B as indicated in the B16F10 melanoma model.

FIG. 12A-FIG. 12D show tumor volume over time (12A) and levels of the indicated enzymes (12B-D) for mice treated with vehicle or Construct B as indicated in the RM-1 prostate cancer model.

FIG. 13A shows MMP activity, measured as described for FIG. 9, in the indicated groups.

FIG. 13B-FIG. 13C show tumor volume over time for mice treated with vehicle or Construct B as indicated in the indicated cancer models.

### DETAILED DESCRIPTION OF CERTAIN EMBODIMENTS

This specification describes exemplary embodiments and applications of the disclosure. The disclosure, however, is not limited to these exemplary embodiments and applications or to the manner in which the exemplary embodiments and applications operate or are described herein. The term “or” is used in an inclusive sense, i.e., equivalent to “and/or,” unless the context dictates otherwise. It is noted that, as used in this specification and the appended claims, the singular forms “a,” “an,” and “the,” and any singular use of any word, include plural referents unless expressly and unequivocally limited to one referent. As used herein, the terms “comprise,” “include,” and grammatical variants thereof are intended to be non-limiting, such that recitation of items in a list is not to the exclusion of other like items that can be substituted or added to the listed items. Section divisions in the specification are provided for the convenience of the reader only and do not limit any combination of elements discussed. In case of any contradiction or conflict between material incorporated by reference and the expressly described content provided herein, the expressly described content controls.

### Overview

Provided herein are protease-activated pro-cytokines (also referred to herein as cytokine prodrugs) comprising a linker comprising a protease-cleavable linker. The cleavable linker can be between a cytokine polypeptide sequence and an inhibitory polypeptide sequence, such that the ability of the cytokine polypeptide sequence to activate immune cells is reduced or eliminated compared to a free cytokine polypeptide sequence. Proteolysis of the linker can liberate the cytokine so that it can activate immune cells.

In some embodiments, the protease-cleavable linker is cleavable by a protease expressed at higher levels in the tumor microenvironment (TME) than in healthy tissue of the same type. In some embodiments, the protease-cleavable linker is a matrix metalloprotease (MMP)-cleavable linker, such as any of the MMP-cleavable linkers described herein. Without wishing to be bound by any particular theory, increased expression of proteases, including but not necessarily limited to MMPs, in the tumor microenvironment (TME) can provide a mechanism for achieving selective or preferential activation of the cytokine prodrug at or near a tumor site. Certain protease-cleavable linkers described herein are considered particularly suitable for achieving such selective or preferential activation.

In any of the foregoing embodiments, the cytokine prodrug may further comprise a pharmacokinetic modulator, e.g., which extends the half-life of the prodrug and which may optionally also extend the half-life of the active cytokine.

Sequences of exemplary cytokine prodrugs and components thereof are shown in Table 1. In Table 1, “X_{Hy}” designates a hydrophobic amino acid residue. In some embodiments, the hydrophobic amino acid residue is any one of glycine (Gly), alanine (Ala), valine (Val), leucine (Leu), isoleucine (Ile), proline (Pro), phenylalanine (Phe), methionine (Met), and tryptophan (Trp). In some embodiments, the hydrophobic amino acid residue is any one of Ala, Leu, Val, Ile, Pro, Phe, Met, and Trp. In some embodiments, the hydrophobic amino acid residue is any one of Leu, Val, Ile, Pro, Phe, Met, and Trp. In some embodiments, the hydrophobic amino acid residue is any one of Ala, Leu, Val, Ile, Phe, Met, and Trp. In some embodiments, the hydrophobic amino acid residue is any one of Leu, Val, Ile, Phe, Met, and Trp. “(Pip)” represents piperidine. “(Hof)” represents homophenylalanine. “(Cit)” represents citrulline. “(Et)” represents ethionine. “C(me)” represents methylcysteine. In certain sequences, underlining is used to indicate mutated positions.

This disclosure further provides uses of these cytokine prodrugs, e.g., for treating cancer. In some embodiments, the cytokine prodrug is selectively or preferentially cleaved in the tumor microenvironment, which may result in beneficial effects, e.g., improved recruitment and/or activation of immune cells in the vicinity of the tumor, and/or reduced systemic exposure to active cytokines.

TABLE 1
Table of Sequences of Cytokine Prodrugs and Components Thereof

| | | | | | |
|---|---|---|---|---|---|
| SEQ | | | | | |
| ID NO | Description | Sequence | Species | Function | Notes |

IL-2 sequences

| | | | | | |
|---|---|---|---|---|---|
| 1 | hIL-2 | APTSSSTKKTQLQLEHLLLDLQM | human | cytokine | wild-type |
| | | ILNGINNYKNPKLTRMLTFKFYM | | | |
| | | PKKATELKHLQCLEEELKPLEEV | | | |
| | | LNLAQSKNFHLRPRDLISNINVI | | | |
| | | VLELKGSETTFMCEYADETATIV | | | |
| | | EFLNRWITFCQSIISTLT | | | |
| 2 | hIL-2 | APTSSSTKKTQLQLEHLLLDLQM | human | cytokine | C125 to |
| | (C125S) | ILNGINNYKNPKLTRMLTFKFYM | | | S |
| | | PKKATELKHLQCLEEELKPLEEV | | | mutation |
| | | LNLAQSKNFHLRPRDLISNINVI | | | |
| | | VLELKGSETTFMCEYADETATIV | | | |
| | | EFLNRWITFSQSIISTLT | | | |
| 3 | mIL-2 | APTSSSTSSSTAEAQQQQQQQQQ | mouse | cytokine | wild-type |
| | | QQQHLEQLLMDLQELLSRMENYR | | | |
| | | NLKLPRMLTFKFYLPKQATELKD | | | |
| | | LQCLEDELGPLRHVLDLTQSKSF | | | |
| | | QLEDAENFISNIRVTVVKLKGSD | | | |
| | | NTFECQFDDESATVVDFLRRWIA | | | |
| | | FCQSIISTSPQ | | | |
| 4 | mIL-2 | APTSSSTSSSTAEAQQQQQQQQQ | mouse | cytokine | C140 to |
| | (C140S) | QQQHLEQLLMDLQELLSRMENYR | | | S |
| | | NLKLPRMLTFKFYLPKQATELKD | | | mutation |
| | | LQCLEDELGPLRHVLDLTQSKSF | | | |
| | | QLEDAENFISNIRVTVVKLKGSD | | | |
| | | NTFECQFDDESATVVDFLRRWIA | | | |
| | | FSQSIISTSPQ | | | |
| 5-9 | Not Used | | | | |

Blockers: IL-2R sequences

| | | | | | |
|---|---|---|---|---|---|
| 10 | hIL-2Ralpha | ELCDDDPPEIPHATFKAMAYKEG | human | blocker | wild-type |
| | | TMLNCECKRGFRRIKSGSLYMLC | | | amino |
| | | TGNSSHSSWDNQCQCTSSATRNT | | | acids 1- |
| | | TKQVTPQPEEQKERKTTEMQSPM | | | 219 |
| | | QPVDQASLPGHCREPPPWENEAT | | | |
| | | ERIYHFVVGQMVYYQCVQGYRAL | | | |
| | | HRGPAESVCKMTHGKTRWTQPQL | | | |
| | | ICTGEMETSQFPGEEKPQASPEG | | | |
| | | RPESETSCLVTTTDFQIQTEMAA | | | |
| | | TMETSIFTTEYQ | | | |
| 11 | hIL-2Ralpha | ELCDDDPPEIPHATFKAMAYKEG | human | Mocker | sushi |
| | (1-63) | TMLNCECKRGFRRIKSGSLYMLC | | | domain 1 |
| | | TGNSSHSSWDNQCQCTS | | | wild-type |
| 12 | hIL-2Ralpha | ELCDDDPPEIPHATFKAMAYKEG | human | blocker | M25 to I |
| | (M25I) | TILNCECKRGFRRIKSGSLYMLC | | | mutation |
| | | TGNSSHSSWDNQCQCTSSATRNT | | | |
| | | TKQVTPQPEEQKERKTTEMQSPM | | | |
| | | QPVDQASLPGHCREPPPWENEAT | | | |
| | | ERIYHFVVGQMVYYQCVQGYRAL | | | |
| | | HRGPAESVCKMTHGKTRWTQPQL | | | |
| | | ICTGEMETSQFPGEEKPQASPEG | | | |
| | | RPESETSCLVTTTDFQIQTEMAA | | | |
| | | TMETSIFTTEYQ | | | |
| 13 | hIL-2Ralpha | ELCDDDPPEIPHATFKAMAYKEG | human | blocker | L42 to V |
| | (L42V) | TMLNCECKRGFRRIKSGSVYMLC | | | mutation |
| | | TGNSSHSSWDNQCQCTSSATRNT | | | |
| | | TKQVTPQPEEQKERKTTEMQPM | | | |
| | | QPVDQASLPGHCREPPPWENEAT | | | |
| | | ERIYHFVVGQMVYYQCVQGYRAL | | | |
| | | HRGPAESVCKMTHGKTRWTQPQL | | | |
| | | ICTGEMETSQFPGEEKPQASPEG | | | |
| | | RPESETSCLVTTTDFQIQTEMAA | | | |
| | | TMETSIFTTEYQ | | | |
| 14 | hIL-2Ralpha | ELCDDDPPEIPHATFKAMAYKEG | human | blocker | M25 to I |
| | (M251 L42V) | TILNCECKRGFRRIKSGSVYMLC | | | mutation; |
| | | TGNSSHSSWDNQCQCTSSATRNT | | | L42 to V |
| | | TKQVTPQPEEQKERKTTEMQSPM | | | mutation |
| | | QPVDQASLPGHCREPPPWENEAT | | | |
| | | ERIYHFVVGQMVYYQCVQGYRAL | | | |
| | | HRGPAESVCKMTHGKTRWTQPQL | | | |
| | | ICTGEMETSQFPGEEKPQASPEG | | | |
| | | RPESETSCLVTTTDFQIQTEMAA | | | |
| | | TMETSIFTTEYQ | | | |
| 15 | Human | LNTTILTPNGNEDTTADFFLTTM | human | blocker | |
| | IL2Rgamma | PTDSLSVSTLPLPEVQCFVFNVE | | | |
| | polypeptide | YMNCTWNSSSEPQPTNLTLHYWY | | | |
| | sequence | KNSDNDKVQKCSHYLFSEEITSG | | | |
| | | CQLQKKEIHLYQTFVVQLQDPRE | | | |
| | | PRRQATQMLKLQNLVIPWAPENL | | | |
| | | TLHKLSESQLELNWNNRFLNHCL | | | |
| | | EHLVQYRTDWDHSWTEQSVDYRH | | | |
| | | KFSLPSVDGQKRYTFRVRSRFNP | | | |
| | | LCGSAQHWSEWSHPIHWGSNTSK | | | |
| | | ENPFLFALEA | | | |
| 16 | Human | AVNGTSQFTCFYNSRANISCVWS | human | blocker | |
| | IL2Rbeta | QDGALQDTSCQVHAWPDRRRWNQ | | | |
| | polypeptide | TCELLPVSQASWACNLILGAPDS | | | |
| | sequence | QKLTTVDIVTLRVLCREGVRWRV | | | |
| | | MAIQDFKPFENLRLMAPISLQVV | | | |
| | | HVETHRCNISWEISQASHYFERH | | | |
| | | LEFEARTLSPGHTWEEAPLLTLK | | | |
| | | QKQEWICLETLTPDTQYEFQVRV | | | |
| | | KPLQGEFTTWSPWSQPLAFRTKP | | | |
| | | AALGKDT | | | |
| 17 | chimeric IL- | ELCLYDPPEVPNATFKALSYKNG | human/ | blocker | mouse |
| | 2Ralpha | TILNCECKRGFRRLKELVYMRCL | mouse | | IL2Ralpha |
| | | GNSWSSNCQCTSSATRNTTKQVT | | | (1-58)- |
| | | PQPEEQKERKTTEMQSPMQPVDQ | | | hu |
| | | ASLPGHCREPPPWENEATERIYH | | | IL2Ralpha |
| | | FVVGQMVYYQCVQGYRALHRGPA | | | (64-219) |
| | | ESVCKMTHGKTRWTQPQLICTGE | | | |
| | | METSQFPGEEKPQASPEGRPESE | | | |
| | | TSCLVTTTDFQIQTEMAATMETS | | | |
| | | IFTTEYQ | | | |
| 18 | mIL-2Ralpha | ELCLYDPPEVPNATFKALSYKNG | mouse | blocker | wild-type |
| | | TILNCECKRGFRRLKELVYMRCL | | | amino |
| | | GNSWSSNCQCTSNSHDKSRKQVT | | | acids 1- |
| | | AQLEHQKEQQTTTDMQKPTQSMH | | | 215 |
| | | QENLTGHCREPPPWKHEDSKRIY | | | |
| | | HFVEGQSVHYECIPGYKALQRGP | | | |
| | | AISICKMKCGKTGWTQPQLTCVD | | | |
| | | EREHHRFLASEESQGSRNSSPES | | | |
| | | ETSCPITTTDFPQPTETTAMTET | | | |
| | | FVLTMEYK | | | |
| 19 | mIL-2Ralpha | ELCLYDPPEVPNATFKALSYKNG | mouse | blocker | sushi |
| | (1-58) | TILNCECKRGFRRLKELVYMRCL | | | domain 1 |
| | | GNSWSSNCQCTS | | | wild-type |
| 20 | hIL-2Ralpha | ELCLYDPPEIPHATFKAMAYKEG | human | blocker | D4 to L |
| | (1-219) | TILNCECKRGFRRIKSGSLYMLC | | | mutation; |
| | M25I/D4L/D5Y | TGNSSHSSWDNQCQCTSSATRNT | | | D5 to Y |
| | | TKQVTPQPEEQKERKTTEMQSPM | | | mutation; |
| | | QPVDQASLPGHCREPPPWENEAT | | | M25 to I |
| | | ERIYHFVVGQMVYYQCVQGYRAL | | | mutation |
| | | HRGPAESVCKMTHGKTRWTQPQL | | | |
| | | ICTGEMETSQFPGEEKPQASPEG | | | |
| | | RPESETSCLVTTTDFQ | | | |
| | | IQTEMAATMETSIFTTEYQ | | | |
| 21 | hIL-2Ralpha | ELCLYDPPEIPHATFKAMAYKEG | human | blocker | D4 to L |
| | (1-219) | TMLNCECKRGFRRIKSGSVYMLC | | | mutation; |
| | L42V/D4L/D5Y | TGNSSHSSWDNQCQCTSSATRNT | | | D5 to Y |
| | | TKQVTPQPEEQKERKTTEMQSPM | | | mutation; |
| | | QPVDQASLPGHCREPPPWENEAT | | | L42 to V |
| | | ERIYHFVVGQMVYYQCVQGYRAL | | | mutation |
| | | HRGPAESVCKMTHGKTRWTQPQL | | | |
| | | ICTGEMETSQFPGEEKPQASPEG | | | |
| | | RPESETSCLVTTTDFQ | | | |
| | | IQTEMAATMETSIFTTEYQ | | | |
| 22 | hIL-2Ralpha | ELCLYDPPEIPHATFKAMAYKEG | human | blocker | D4 to L |
| | (1-219) | TILNCECKRGFRRIKSGSVYMLC | | | mutation; |
| | M25I/L42V/D4L/ | TGNSSHSSWDNQCQCTSSATRNT | | | D5 to Y |
| | D5Y | TKQVTPQPEEQKERKTTEMQSPM | | | mutation; |
| | | QPVDQASLPGHCREPPPWENEAT | | | M25 to I |
| | | ERIYHFVVGQMVYYQCVQGYRAL | | | mutation; |
| | | HRGPAESVCKMTHGKTRWTQPQL | | | L42 to V |
| | | ICTGEMETSQFPGEEKPQASPEG | | | mutation |
| | | RPESETSCLVTTTDFQ | | | |
| | | IQTEMAATMETSIFTTEYQ | | | |
| 23 | hIL-2Ralpha | ELCLYDPPEIPHATFKAMAYKEG | human | blocker | D4 to L |
| | (1-219) | TMLNCECKRGFRRIKSGSLYMLC | | | mutation; |
| | D4L/D5Y | TGNSSHSSWDNQCQCTSSATRNT | | | D5 to Y |
| | | TKQVTPQPEEQKERKTTEMQSPM | | | mutation |
| | | QPVDQASLPGHCREPPPWENEAT | | | |
| | | ERIYHFVVGQMVYYQCVQGYRAL | | | |
| | | HRGPAESVCKMTHGKTRWTQPQL | | | |
| | | ICTGEMETSQFPGEEKPQASPEG | | | |
| | | RPESETSCLVTTTDFQ | | | |
| | | IQTEMAATMETSIFTTEYQ | | | |
| 24 | hIL-2Ralpha | ELCDDDPPEIPHATFKAMAYKEG | human | blocker | Wild- |
| | (1-219) | TMLNCECKRGFRRIKELVYMLCT | | | type |
| | SGSL₃₉₋₄₂ | GNSSHSSWDNQCQCTSSATRNTT | | | residues |
| | ELV | KQVTPQPEEQKERKTTEMQSPMQ | | | 39-42 |
| | | PVDQASLPGHCREPPPWENEATE | | | replaced |
| | | RIYHFVVGQMVYYQCVQGYRALH | | | with ELV |
| | | RGPAESVCKMTHGKTRWTQPQLI | | | |
| | | CTGEMETSQFPGEEKPQASPEGR | | | |
| | | PESETSCLVTTTDFQ | | | |
| | | IQTEMAATMETSIFTTEYQ | | | |
| 25 | hIL-2Ralpha | ELCDDDPPEIPHATFKAMAYKEG | human | blocker | Wild- |
| | (1-192) | TMLNCECKRGFRRIKSGSLYMLC | | | type |
| | | TGNSSHSSWDNQCQCTSSATRNT | | | amino |
| | | TKQVTPQPEEQKERKTTEMQSPM | | | acids 1- |
| | | QPVDQASLPGHCREPPPWENEAT | | | 192 |
| | | ERIYHFVVGQMVYYQCVQGYRAL | | | |
| | | HRGPAESVCKMTHGKTRWTQPQL | | | |
| | | ICTGEMETSQFPGEEKPQASPEG | | | |
| | | RPESETSC | | | |
| 26 | hIL-2Ralpha | ELCDDDPPEIPHATFKAMAYKEG | human | blocker | M25 to I |
| | (1-192) M25I | TILNCECKRGFRRIKSGSLYMLC | | | mutation |
| | | TGNSSHSSWDNQCQCTSSATRNT | | | |
| | | TKQVTPQPEEQKERKTTEMQSPM | | | |
| | | QPVDQASLPGHCREPPPWENEAT | | | |
| | | ERIYHFVVGQMVYYQCVQGYRAL | | | |
| | | HRGPAESVCKMTHGKTRWTQPQL | | | |
| | | ICTGEMETSQFPGEEKPQASPEG | | | |
| | | RPESETSC | | | |
| 27 | hIL-2Ralpha | ELCDDDPPEIPHATFKAMAYKEG | human | blocker | L42 to V |
| | (1-192) L42V | TMLNCECKRGFRRIKSGSVYMLC | | | mutation |
| | | TGNSSHSSWDNQCQCTSSATRNT | | | |
| | | TKQVTPQPEEQKERKTTEMQSPM | | | |
| | | QPVDQASLPGHCREPPPWENEAT | | | |
| | | ERIYHFVVGQMVYYQCVQGYRAL | | | |
| | | HRGPAESVCKMTHGKTRWTQPQL | | | |
| | | ICTGEMETSQFPGEEKPQASPEG | | | |
| | | RPESETSC | | | |
| 28 | hIL-2Ralpha | ELCLYDPPEIPHATFKAMAYKEG | human | blocker | D4 to L |
| | (1-192) | TMLNCECKRGFRRIKSGSLYMLC | | | mutation; |
| | D4L/D5Y | TGNSSHSSWDNQCQCTSSATRNT | | | D5 to Y |
| | | TKQVTPQPEEQKERKTTEMQSPM | | | mutation |
| | | QPVDQASLPGHCREPPPWENEAT | | | |
| | | ERIYHFVVGQMVYYQCVQGYRAL | | | |
| | | HRGPAESVCKMTHGKTRWTQPQL | | | |
| | | ICTGEMETSQFPGEEKPQASPEG | | | |
| | | RPESETSC | | | |
| 29 | hIL-2Ralpha | ELCDDDPPEIPHATFKAMAYKEG | human | blocker | Wild- |
| | (1-192) | TMLNCECKRGFRRIKELVYMLCT | | | type |
| | SGSL₃₉₋₄₂ | GNSSHSSWDNQCQCTSSATRNTT | | | residues |
| | ELV | KQVTPQPEEQKERKTTEMQSPMQ | | | 39-42 |
| | | PVDQASLPGHCREPPPWENEATE | | | replaced |
| | | RIYHFVVGQMVYYQCVQGYRALH | | | with ELV |
| | | RGPAESVCKMTHGKTRWTQPQLI | | | |
| | | CTGEMETSQFPGEEKPQASPEGR | | | |
| | | PESETSC | | | |

IL2 Blockers: anti-IL2 sequences

| | | | | | |
|---|---|---|---|---|---|
| 30 | scFv2 | QSVLTQPPSVSGAPGQRVTISCT | human | blocker | wild-type |
| | | GTSSNIGAHYDVHWYQQFPGTAP | | | |
| | | KRLIYGNNNRPSGVPARFSGSKS | | | |
| | | GTSASLAITGLQAEDEADYYCQS | | | |
| | | YDRSLRGWVFGGGTKLTVLGEGK | | | |
| | | SSGSGSESKASEVQLVESGGGLV | | | |
| | | QPGRSLRLSCAASGFTFDDYAMH | | | |
| | | WVRQAPGKGLEWVSGISWNSGSI | | | |
| | | GYADSVKGRFTISRDNSKNTLYL | | | |
| | | QMNSLRAEDTAVYYCAKDVNWNY | | | |
| | | GYYFDYWGQGTLVTVSS | | | |
| 31 | scFv2 (18mer | QSVLTQPPSVSGAPGQRVTISCT | human | blocker | 18 mer |
| | linker) | GTSSNIGAHYDVHWYQQFPGTAP | | | linker |
| | | KRLIYGNNNRPSGVPARFSGSKS | | | between |
| | | GTSASLAITGLQAEDEADYYCQS | | | VL and VH |
| | | YDRSLRGWVFGGGTKLTVLGGST | | | |
| | | SGSGKPGSGEGSTKGEVQLVESG | | | |
| | | GGLVQPGRSLRLSCAASGFTFDD | | | |
| | | YAMHWVRQAPGKGLEWVSGISWN | | | |
| | | SGSIGYADSVKGRFTISRDNSKN | | | |
| | | TLYLQMNSLRAEDTAVYYCAKDV | | | |
| | | NWNYGYYFDYWGQGTLVTVSS | | | |
| 32 | VL domain of | QSVLTQPPSVSGAPGQRVTISCT | human | blocker | wild-type |
| | scFv2 | GTSSNIGAHYDVHWYQQFPGTAP | | | |
| | | KRLIYGNNNRPSGVPARFSGSKS | | | |
| | | GTSASLAITGLQAEDEADYYCQS | | | |
| | | YDRSLRGWVFGGGTKLTVLG | | | |
| 33 | VH domain of | EVQLVESGGGLVQPGRSLRLSCA | human | blocker | wild-type |
| | scFv2 | ASGFTFDDYAMHWVRQAPGKGLE | | | |
| | | WVSGISWNSGSIGYADSVKGRFT | | | |
| | | ISRDNSKNTLYLQMNSLRAEDTA | | | |
| | | VYYCAKDVNWNYGYYFDYWGQGT | | | |
| | | LVTVSS | | | |
| 34 | scFv2 VL | TGTSSNIGAHYDVH | | | |
| | HVR1 | | | | |
| 35 | scFv2 VL | GNNNRPS | | | |
| | HVR2 | | | | |
| 36 | scFv2 VL | QSYDRSLRGWV | | | |
| | HVR3 | | | | |
| 37 | scFv2 VH | DDYAMH | | | |
| | HVR1 | | | | |
| 38 | scFv2 VH | GISWNSGSIGYADSVKG | | | |
| | HVR2 | | | | |
| 39 | scFv2 VH | KDVNWNYGYYFDY | | | |
| | HVR3 | | | | |
| 247 | scFv183 | DIVMTQSPDSLAVSLGERATINC | human | blocker | linker |
| | | KSSQSVLYSNNNKNYLAWYQQKP | | | between |
| | | GQPPKLLIYGASTRESWVPDRFS | | | VL and |
| | | GSGSGTDFTLTISSLQAEDVAVY | | | VH |
| | | YCQQWYYYPYTFGQGTKVEIKGG | | | |
| | | GGSGGGGSGGGSGGGGSEVQLLE | | | |
| | | SGGGLVQPGGSLRLSCAASGFTF | | | |
| | | SSYYMSWVRQAPGKGLEWVSDIS | | | |
| | | GRGGQTNYADSVKGRFTISRDNS | | | |
| | | KNTLYLQMNSLRAEDTAVYYCAR | | | |
| | | GGGSFANWGRGTLVTVSS | | | |
| 248 | VH domain of | DIVMTQSPDSLAVSLGERATINC | human | blocker | |
| | scFv2 | KSSQSVLYSNNNKNYLAWYQQKP | | | |
| | | GQPPKLLIYGASTRESWVPDRFS | | | |
| | | GSGSGTDFTLTISSLQAEDVAVY | | | |
| | | YCQQWYYYPYTFGQGTKVEIK | | | |
| 249 | VL domain of | EVQLLESGGGLVQPGGSLRLSCA | human | blocker | |
| | scFv2 | ASGFTFSSYYMSWVRQAPGKGLE | | | |
| | | WVSDISGRGGQTNYADSVKGRFT | | | |
| | | ISRDNSKNTLYLQMNSLRAEDTA | | | |
| | | VYYCARGGGSFANWGRGTLVTVS | | | |
| | | S | | | |
| 250 | scFv2 VL | KSSQSVLYSNNNKNYLA | | | |
| | HVR1 | | | | |
| 251 | scFv2 VL | GASTRES | | | |
| | HVR2 | | | | |
| 252 | scFv2 VL | QQWYYYPYT | | | |
| | HVR3 | | | | |
| 253 | scFv2 VH | SSYYMS | | | |
| | HVR1 | | | | |
| 254 | scFv2 VH | DISGRGGQTNYADSVKG | | | |
| | HVR2 | | | | |
| 255 | scFv2 VH | RGGGSFAN | | | |
| | HVR3 | | | | |

Blockers: IL-2R sequences

| | | | | | |
|---|---|---|---|---|---|
| 40 | hIL-2Ralpha | ELCLYDPPEIPHATFKAMAYKEG | human | blocker | D4 to L |
| | (1-192) | TILNCECKRGFRRIKSGSLYMLC | | | mutation; |
| | M25I/D4L/ | TGNSSHSSWDNQCQCTSSATRNT | | | D5 to Y |
| | D5Y | TKQVTPQPEEQKERKTTEMQSPM | | | mutation; |
| | | QPVDQASLPGHCREPPPWENEAT | | | M25 to I |
| | | ERIYHFVVGQMVYYQCVQGYRAL | | | mutation |
| | | HRGPAESVCKMTHGKTRWTQPQL | | | |
| | | ICTGEMETSQFPGEEKPQASPEG | | | |
| | | RPESETSC | | | |
| 41 | hIL-2Ralpha | ELCDDDPPEIPHATFKAMAYKEG | human | blocker | M25 to I |
| | (1-192) | TILNCECKRGFRRIKSGSVYMLC | | | mutation; |
| | M25I/L42V | TGNSSHSSWDNQCQCTSSATRNT | | | L42 to V |
| | | TKQVTPQPEEQKERKTTEMQSPM | | | mutation |
| | | QPVDQASLPGHCREPPPWENEAT | | | |
| | | ERIYHFVVGQMVYYQCVQGYRAL | | | |
| | | HRGPAESVCKMTHGKTRWTQPQL | | | |
| | | ICTGEMETSQFPGEEKPQASPEG | | | |
| | | RPESETSC | | | |
| 42 | hIL-2Ralpha | ELCLYDPPEIPHATFKAMAYKEG | human | blocker | D4 to L |
| | (1-192) | TMLNCECKRGFRRIKSGSVYMLC | | | mutation; |
| | D4L/D5Y/L42V | TGNSSHSSWDNQCQCTSSATRNT | | | D5 to Y |
| | | TKQVTPQPEEQKERKTTEMQSPM | | | mutation; |
| | | QPVDQASLPGHCREPPPWENEAT | | | L42 to V |
| | | ERIYHFVVGQMVYYQCVQGYRAL | | | mutation |
| | | HRGPAESVCKMTHGKTRWTQPQL | | | |
| | | ICTGEMETSQFPGEEKPQASPEG | | | |
| | | RPESETSC | | | |
| 43 | hIL-2Ralpha | ELCLYDPPEIPHATFKAMAYKEG | human | blocker | D4 to L |
| | (1-192) | TILNCECKRGFRRIKSGSVYMLC | | | mutation; |
| | M25I/D4L/D5Y/ | TGNSSHSSWDNQCQCTSSATRNT | | | D5 to Y |
| | L42V | TKQVTPQPEEQKERKTTEMQSPM | | | mutation; |
| | | QPVDQASLPGHCREPPPWENEAT | | | M25 to I |
| | | ERIYHFVVGQMVYYQCVQGYRAL | | | mutation; |
| | | HRGPAESVCKMTHGKTRWTQPQL | | | L42 to V |
| | | ICTGEMETSQFPGEEKPQASPEG | | | mutation |
| | | RPESETSC | | | |
| 44 | hIL-2Ralpha | ELCDDDPPEIPHATFKAMAYKEG | human | blocker | Wild- |
| | (1-178) | TMLNCECKRGFRRIKSGSLYMLC | | | type |
| | | TGNSSHSSWDNQCQCTSSATRNT | | | amino |
| | | TKQVTPQPEEQKERKTTEMQSPM | | | acids 1- |
| | | QPVDQASLPGHCREPPPWENEAT | | | 178 |
| | | ERIYHFVVGQMVYYQCVQGYRAL | | | |
| | | HRGPAESVCKMTHGKTRWTQPQL | | | |
| | | ICTGEMETSQFPGEEKP | | | |
| 45 | hIL-2Ralpha | ELCDDDPPEIPHATFKAMAYKEG | human | blocker | M25 to I |
| | (1-178) M25I | TILNCECKRGFRRIKSGSLYMLC | | | mutation |
| | | TGNSSHSSWDNQCQCTSSATRNT | | | |
| | | TKQVTPQPEEQKERKTTEMQSPM | | | |
| | | QPVDQASLPGHCREPPPWENEAT | | | |
| | | ERIYHFVVGQMVYYQCVQGYRAL | | | |
| | | HRGPAESVCKMTHGKTRWTQPQL | | | |
| | | ICTGEMETSQFPGEEKP | | | |
| 46 | hIL-2Ralpha | ELCDDDPPEIPHATFKAMAYKEG | human | blocker | L42 to V |
| | (1-178) L42V | TMLNCECKRGFRRIKSGSVYMLC | | | mutation |
| | | TGNSSHSSWDNQCQCTSSATRNT | | | |
| | | TKQVTPQPEEQKERKTTEMQSPM | | | |
| | | QPVDQASLPGHCREPPPWENEAT | | | |
| | | ERIYHFVVGQMVYYQCVQGYRAL | | | |
| | | HRGPAESVCKMTHGKTRWTQPQL | | | |
| | | ICTGEMETSQFPGEEKP | | | |
| 47 | hIL-2Ralpha | ELCLYDPPEIPHATFKAMAYKEG | human | blocker | D4 to L |
| | (1-178) | TMLNCECKRGFRRIKSGSLYMLC | | | mutation; |
| | D4L/D5Y | TGNSSHSSWDNQCQCTSSATRNT | | | D5 to Y |
| | | TKQVTPQPEEQKERKTTEMQSPM | | | mutation |
| | | QPVDQASLPGHCREPPPWENEAT | | | |
| | | ERIYHFVVGQMVYYQCVQGYRAL | | | |
| | | HRGPAESVCKMTHGKTRWTQPQL | | | |
| | | ICTGEMETSQFPGEEKP | | | |
| 48 | hIL-2Ralpha | ELCDDDPPEIPHATFKAMAYKEG | human | blocker | Wild- |
| | (1-178) | TMLNCECKRGFRRIKELVYMLCT | | | type |
| | SGSL39-42 | GNSSHSSWDNQCQCTSSATRNTT | | | residues |
| | ELV | KQVTPQPEEQKERKTTEMQSPMQ | | | 39-42 |
| | | PVDQASLPGHCREPPPWENEATE | | | replaced |
| | | RIYHFVVGQMVYYQCVQGYRALH | | | with ELV |
| | | RGPAESVCKMTHGKTRWTQPQLI | | | |
| | | CTGEMETSQFPGEEKP | | | |
| 49 | hIL-2Ralpha | ELCDDDPPEIPHATFKAMAYKEG | human | blocker | M25 to I |
| | (1-178) | TILNCECKRGFRRIKSGSVYMLC | | | mutation; |
| | M25I/L42V | TGNSSHSSWDNQCQCTSSATRNT | | | L42 to V |
| | | TKQVTPQPEEQKERKTTEMQSPM | | | mutation |
| | | QPVDQASLPGHCREPPPWENEAT | | | |
| | | ERIYHFVVGQMVYYQCVQGYRAL | | | |
| | | HRGPAESVCKMTHGKTRWTQPQL | | | |
| | | ICTGEMETSQFPGEEKP | | | |
| 50 | hIL-2Ralpha | ELCLYDPPEIPHATFKAMAYKEG | human | blocker | D4 to L |
| | (1-178) | TMLNCECKRGFRRIKSGSVYMLC | | | mutation; |
| | D4L/D5Y/L42V | TGNSSHSSWDNQCQCTSSATRNT | | | D5 to Y |
| | | TKQVTPQPEEQKERKTTEMQSPM | | | mutation; |
| | | QPVDQASLPGHCREPPPWENEAT | | | L42 to V |
| | | ERIYHFVVGQMVYYQCVQGYRAL | | | mutation |
| | | HRGPAESVCKMTHGKTRWTQPQL | | | |
| | | ICTGEMETSQFPGEEKP | | | |
| 51 | hIL-2Ralpha | ELCLYDPPEIPHATFKAMAYKEG | human | blocker | D4 to L |
| | (1-178) | TILNCECKRGFRRIKSGSVYMLC | | | mutation; |
| | D4L/D5Y/M25I/ | TGNSSHSSWDNQCQCTSSATRNT | | | D5 to Y |
| | L42V | TKQVTPQPEEQKERKTTEMQSPM | | | mutation; |
| | | QPVDQASLPGHCREPPPWENEAT | | | M25 to I |
| | | ERIYHFVVGQMVYYQCVQGYRAL | | | mutation; |
| | | HRGPAESVCKMTHGKTRWTQPQL | | | L42 to V |
| | | ICTGEMETSQFPGEEKP | | | mutation |
| 52-69 | Not Used | | | | |

Pharmacokinetic modulators

| | | | | | |
|---|---|---|---|---|---|
| 70 | hIgG1 Fc | DKTHTCPPCPAPELLGGPSVFLF | human | half-life | C- |
| | | PPKPKDTLMISRTPEVTCVVVDV | | extension | terminal |
| | | SHEDPEVKFNWYVDGVEVHNAKT | | | K residue |
| | | KPREEQYNSTYRVVSVLTVLHQD | | | deleted |
| | | WLNGKEYKCKVSNKALPAPIEKT | | | |
| | | ISKAKGQPREPQVYTLPPSRDEL | | | |
| | | TKNQVSLTCLVKGFYPSDIAVEW | | | |
| | | ESNGQPENNYKTTPPVLDSDGSF | | | |
| | | FLYSKLTVDKSRWQQGNVFSCSV | | | |
| | | MHEALHNHYTQKSLSLSPG | | | |
| 71 | Human IgG1 | DKTHTCPPCPAPELLGGPSVFLF | human | half-life | |
| | K392D | PPKPKDTLMISRTPEVTCVVVDV | | extension | |
| | K409D Fc | SHEDPEVKFNWYVDGVEVHNAKT | | | |
| | domain | KPREEQYNSTYRVVSVLTVLHQD | | | |
| | polypeptide | WLNGKEYKCKVSNKALPAPIEKT | | | |
| | sequence | ISKAKGQPREPQVYTLPPSRDEL | | | |
| | | TKNQVSLTCLVKGFYPSDIAVEW | | | |
| | | ESNGQPENNYDTTPPVLDSDGSF | | | |
| | | FLYSDLTVDKSRWQQGNVFSCSV | | | |
| | | MHEALHNHYTQKSLSLSPG | | | |
| 72 | Human serum | RGVFRRDAHKSEVAHRFKDLGEE | human | half-life | wild-type |
| | albumin | NFKALVLIAFAQYLQQCPFEDHV | | extension | |
| | | KLVNEVTEFAKTCVADESAENCD | | | |
| | | KSLHTLFGDKLCTVATLRETYGE | | | |
| | | MADCCAKQEPERNECFLQHKDDN | | | |
| | | PNLPRLVRPEVDVMCTAFHDNEE | | | |
| | | TFLKKYLYEIARRHPYFYAPELL | | | |
| | | FFAKRYKAAFTECCQAADKAACL | | | |
| | | LPKLDELRDEGKASSAKQRLKCA | | | |
| | | SLQKFGERAFKAWAVARLSQRFP | | | |
| | | KAEFAEVSKLVTDLTKVHTECCH | | | |
| | | GDLLECADDRADLAKYICENQDS | | | |
| | | ISSKLKECCEKPLLEKSHCIAEV | | | |
| | | ENDEMPADLPSLAADFVESKDVC | | | |
| | | KNYAEAKDVFLGMFLYEYARRHP | | | |
| | | DYSVVLLLRLAKTYETTLEKCCA | | | |
| | | AADPHECYAKVFDEFKPLVEEPQ | | | |
| | | NLIKQNCELFEQLGEYKFQNALL | | | |
| | | VRYTKKVPQVSTPTLVEVSRNLG | | | |
| | | KVGSKCCKHPEAKRMPCAEDYLS | | | |
| | | VVLNQLCVLHEKTPVSDRVTKCC | | | |
| | | TESLVNRRPCFSALEVDETYVPK | | | |
| | | EFNAETFTFHADICTLSEKERQI | | | |
| | | KKQTALVELVKHKPKATKEQLKA | | | |
| | | VMDDFAAFVEKCCKADDKETCFA | | | |
| | | EEGKKLVAASQAALGL | | | |
| 73 | mIgG1 Fc | GCKPCICTVPEVSSVFIFPPKPK | mouse | half-life | wild-type |
| | | DVLTITLTPKVTCVVVDISKDDP | | extension | |
| | | EVQFSWFVDDVEVHTAQTQPREE | | | |
| | | QFNSTFRSVSELPIMHQDWLNGK | | | |
| | | EFKCRVNSAAFPAPIEKTISKTK | | | |
| | | GRPKAPQVYTIPPPKEQMAKDKV | | | |
| | | SLTCMITDFFPEDITVEWQWNGQ | | | |
| | | PAENYKNTQPIMDTDGSYFVYSK | | | |
| | | LNVQKSNWEAGNTFTCSVLHEGL | | | |
| | | HNHHTEKSLSHSPGK | | | |
| 74 | Murine IgG1 | GCKPCICTVPEVSSVFIFPPKPK | mouse | half-life | |
| | T252M Fc | DVLMITLTPKVTCVVVDISKDDP | | extension | |
| | domain | EVQFSWFVDDVEVHTAQTQPREE | | | |
| | polypeptide | QFNSTFRSVSELPIMHQDWLNGK | | | |
| | sequence | EFKCRVNSAAFPAPIEKTISKTK | | | |
| | | GRPKAPQVYTIPPPKEQMAKDKV | | | |
| | | SLTCMITDFFPEDITVEWQWNGQ | | | |
| | | PAENYKNTQPIMDTDGSYFVYSK | | | |
| | | LNVQKSNWEAGNTFTCSVLHEGL | | | |
| | | HNHHTEKSLSHSPG | | | |
| 75-79 | Not Used | | | | |
| 256 | IgG1 Fc | DKTHTCPPCPAPELLGGPSVFLF | human | half-life | Knob |
| | (K360E/K409W) | PPKPKDTLMISRTPEVTCVVVDV | | extension | mutations |
| | Knob | SHEDPEVKFNWYVDGVEVHNAKT | | | |
| | | KPREEQYNSTYRVVSVLTVLHQD | | | |
| | | WLNGKEYKCKVSNKALPAPIEKT | | | |
| | | ISKAKGQPREPQVYTLPPSRDEL | | | |
| | | TENQVSLTCLVKGFYPSDIAVEW | | | |
| | | ESNGQPENNYKTTPPVLDSDGSF | | | |
| | | FLYSWLTVDKSRWQQGNVFSCSV | | | |
| | | MHEALHNHYTQKSLSLSPG | | | |
| 257 | hIgG1 Fc | DKTHTCPPCPAPELLGGPSVFLF | human | half-life | Hole |
| | Q347R/D399V/ | PPKPKDTLMISRTPEVTCVVVDV | | extension | mutations |
| | F405T) | SHEDPEVKFNWYVDGVEVHNAKT | | | |
| | Hole | KPREEQYNSTYRVVSVLTVLHQD | | | |
| | | WLNGKEYKCKVSNKALPAPIEKT | | | |
| | | ISKAKGQPREPRVYTLPPSRDEL | | | |
| | | TKNQVSLTCLVKGFYPSDIAVEW | | | |
| | | ESNGQPENNYKTTPPVLVSDGSF | | | |
| | | TLYSKLTVDKSRWQQGNVFSCSV | | | |
| | | MHEALHNHYTQKSLSLSPG | | | |

MMP cleavable segments

| | | |
|---|---|---|
| 80 | MMP | GPLGVRG |
| | cleavage site | |
| | polypeptide | |
| | sequence | |
| 81 | G112631 | GPLGVRG |
| | polypeptide | |
| | sequence | |
| 82 | G112632 | GPLGLRG |
| | polypeptide | |
| | sequence | |
| 83 | G112633 | GPLGLAR |
| | polypeptide | |
| | sequence | |
| 84 | G112634 | GPAALVGA |
| | polypeptide | |
| | sequence | |
| 85 | G112635 | GPAALIGG |
| | polypeptide | |
| | sequence | |
| 86 | G112636 | GPLNLVGR |
| | polypeptide | |
| | sequence | |
| 87 | G112637 | GPAGLVAD |
| | polypeptide | |
| | sequence | |
| 88 | G112638 | GPANLVAP |
| | polypeptide | |
| | sequence | |
| 89 | G112639 | VPLSLYSG |
| | polypeptide | |
| | sequence | |
| 90 | G112640 | SGESPAYYTA |
| | polypeptide | |
| | sequence | |
| 91 | MMP | PXXX_{Hy} |
| | consensus | |
| | motif | |
| 92 | MMP-2 | (L/I)XXX_{Hy} |
| | consensus | |
| | motif | |
| 93 | MMP-2 | X_{Hy}SXL |
| | consensus | |
| | motif | |
| 94 | MMP-2 | FIXXX_{Hy} |
| | consensus | |
| | motif | |
| 95-99 | Not Used | |

IL-2 Fusion polypeptides

| | | |
|---|---|---|
| 100 | Construct A | APTSSSTSSSTAEAQQQQQQQQQQQQHLEQLLMDLQELLSRMENYRN |
| | polypeptide | LKLPRMLTFKFYLPKQATELKDLQCLEDELGPLRHVLDLTQSKSFQL |
| | sequence: | EDAENFISNIRVTVVKLKGSDNTFECQFDDESATVVDFLRRWIAFCQ |
| | mIL2- | SIISTSPQSGGGGSGGGGGPLGVRGGGGGSGGGGSELCLYDPPEVPN |
| | 2x(SG4)- | ATFKALSYKNGTILNCECKRGFRRLKELVYMRCLGNSWSSNCQCTSN |
| | MMPcs1- | SHDKSRKQVTAQLEHQKEQQTTTDMQKPTQSMHQENLTGHCREPPPW |
| | 2x(G4S)- | KHEDSKRIYHFVEGQSVHYECIPGYKALQRGPAISICKMKCGKTGWT |
| | IL2Ralpha- | QPQLTCVDEREHHRFLASEESQGSRNSSPESETSCPITTTDFPQPTE |
| | 6His | TTAMTETFVLTMEYKHHHHHH |
| 101 | Construct B | APTSSSTSSSTAEAQQQQQQQQQQQQHLEQLLMDLQELLSRMENYRN |
| | polypeptide | LKLPRMLTFKFYLPKQATELKDLQCLEDELGPLRHVLDLTQSKSFQL |
| | sequence: | EDAENFISNIRVTVVKLKGSDNTFECQFDDESATVVDFLRRWIAFCQ |
| | mIL2-2x(SG4)- | SIISTSPQSGGGGSGGGGGPLGVRGGGGGSGGGGSELCLYDPPEVPN |
| | MMPcs1- | ATFKALSYKNGTILNCECKRGFRRLKELVYMRCLGNSWSSNCQCTSN |
| | 2x(G4S)- | SHDKSRKQVTAQLEHQKEQQTTTDMQKPTQSMHQENLTGHCREPPPW |
| | IL2Ralpha- | KHEDSKRIYHFVEGQSVHYECIPGYKALQRGPAISICKMKCGKTGWT |
| | mIgG1 Fc | QPQLTCVDEREHHRFLASEESQGSRNSSPESETSCPITTTDFPQPTE |
| | | TTAMTETFVLTMEYKGCKPCICTVPEVSSVFIFPPKPKDVLTITLTP |
| | | KVTCVVVDISKDDPEVQFSWFVDDVEVHTAQTQPREEQFNSTFRSVS |
| | | ELPIMHQDWLNGKEFKCRVNSAAFPAPIEKTISKTKGRPKAPQVYTI |
| | | PPPKEQMAKDKVSLTCMITDFFPEDITVEWQWNGQPAENYKNTQPIM |
| | | DTDGSYFVYSKLNVQKSNWEAGNTFTCSVLHEGLHNHHTEKSLSHSP |
| | | GK |
| 102 | Construct C | APTSSSTSSSTAEAQQQQQQQQQQQQHLEQLLMDLQELLSRMENYRN |
| | polypeptide | LKLPRMLTFKFYLPKQATELKDLQCLEDELGPLRHVLDLTQSKSFQL |
| | sequence: | EDAENFISNIRVTVVKLKGSDNTFECQFDDESATVVDFLRRWIAFSQ |
| | mIL2(C140S)- | SIISTSPQSGGGGSGGGGGPLGVRGGGGGSGGGGSELCLYDPPEVPN |
| | 2x(SG4)- | ATFKALSYKNGTILNCECKRGFRRLKELVYMRCLGNSWSSNCQCTSN |
| | MMPcs1- | SHDKSRKQVTAQLEHQKEQQTTTDMQKPTQSMHQENLTGHCREPPPW |
| | 2x(G4S)- | KHEDSKRIYHFVEGQSVHYECIPGYKALQRGPAISICKMKCGKTGWT |
| | IL2Ralpha- | QPQLTCVDEREHHRFLASEESQGSRNSSPESETSCPITTTDFPQPTE |
| | mIgG1 | TTAMTETFVLTMEYKGCKPCICTVPEVSSVFIFPPKPKDVLMITLTP |
| | Fc(T252M)- | KVTCVVVDISKDDPEVQFSWFVDDVEVHTAQTQPREEQFNSTFRSVS |
| | 6xHIS | ELPIMHQDWLNGKEFKCRVNSAAFPAPIEKTISKTKGRPKAPQVYTI |
| | | PPPKEQMAKDKVSLTCMITDFFPEDITVEWQWNGQPAENYKNTQPIM |
| | | DTDGSYFVYSKLNVQKSNWEAGNTFTCSVLHEGLHNHHTEKSLSHSP |
| | | GHHHHHH |
| 103 | Construct R | APTSSSTSSSTAEAQQQQQQQQQQQQHLEQLLMDLQELLSRMENYRN |
| | polypeptide | LKLPRMLTFKFYLPKQATELKDLQCLEDELGPLRHVLDLTQSKSFQL |
| | sequence: | EDAENFISNIRVTVVKLKGSDNTFECQFDDESATVVDFLRRWIAFSQ |
| | mIL2(C140S)- | SIISTSPQSGGGGSGGGGGPLGVRGGGGGSGGGGSELCLYDPPEVPN |
| | 2x(SG4)- | ATFKALSYKNGTILNCECKRGFRRLKELVYMRCLGNSWSSNCQCTSN |
| | MMPcs1- | SHDKSRKQVTAQLEHQKEQQTTTDMQKPTQSMHQENLTGHCREPPPW |
| | 2x(G4S)- | KHEDSKRIYHFVEGQSVHYECIPGYKALQRGPAISICKMKCGKTGWT |
| | IL2Ralpha- | QPQLTCVDEREHHRFLASEESQGSRNSSPESETSCPITTTDFPQPTE |
| | hu IgG1 Fc- | TTAMTETFVLTMEYKDKTHTCPPCPAPELLGGPSVFLFPPKPKDTLM |
| | 6xHIS | ISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREP |
| | | QVYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQKS |
| | | LSLSPGHHHHHH |
| 104 | Construct D | APTSSSTKKTQLQLEHLLLDLQMILNGINNYKNPKLTRMLTFKFYMP |
| | polypeptide | KKATELKHLQCLEEELKPLEEVLNLAQSKNFHLRPRDLISNINVIVL |
| | Sequence: | ELKGSETTFMCEYADETATIVEFLNRWITFSQSIISTLTSGGGGSGG |
| | mIL2(C140S)- | GGGPLGVRGGGGGSGGGGSELCDDDPPEIPHATFKAMAYKEGTMLNC |
| | 2x(SG4)- | ECKRGFRRIKSGSLYMLCTGNSSHSSWDNQCQCTSSATRNTTKQVTP |
| | MMPcs1- | QPEEQKERKTTEMQSPMQPVDQASLPGHCREPPPWENEATERIYHFV |
| | 2x(G4S)- | VGQMVYYQCVQGYRALHRGPAESVCKMTHGKTRWTQPQLICTGEMET |
| | sIL2Ralpha- | SQFPGEEKPQASPEGRPESETSCLVTTTDFQIQTEMAATMETSIFTT |
| | mIgG1 | EYQDKTHTCPPCPAPELLGGPSVFLFPPKPKDTLMISRTPEVTCVVV |
| | Fc(T252M)- | DVSHEDPEVKFNWYVDGVEVHNAKTKPREEQYNSTYRVVSVLTVLHQ |
| | 6xHIS | DWLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSFF |
| | | LYSKLTVDKSRWQQGNVFSCSVMHEALHNHYTQKSLSLSPGHHHHHH |
| 105 | mIgG1 Fc- | GCKPCICTVPEVSSVFIFPPKPKDVLTITLTPKVTCVVVDISKDDPE |
| | Murine IL2- | VQFSWFVDDVEVHTAQTQPREEQFNSTFRSVSELPIMHQDWLNGKEF |
| | 2x(SG4)- | KCRVNSAAFPAPIEKTISKTKGRPKAPQVYTIPPPKEQMAKDKVSLT |
| | MMPcs1-2x | CMITDFFPEDITVEWQWNGQPAENYKNTQPIMDTDGSYFVYSKLNVQ |
| | (G4S)- | KSNWEAGNTFTCSVLHEGLHNHHTEKSLSHSPGKAPTSSSTSSSTA |
| | IL2Ralpha | EAQQQQQQQQQQQQHLEQLLMDLQELLSRMENYRNLKLPRMLTFKFY |
| | (long kinetic | LPKQATELKDLQCLEDELGPLRHVLDLTQSKSFQLEDAENFISNIRV |
| | IL2 post | TVVKLKGSDNTFECQFDDESATVVDFLRRWIAFCQSIISTSPQSGGG |
| | cleavage) | GSGGGGGPLGVRGGGGGSGGGGSELCLYDPPEVPNATFKALSYKNGT |
| | polypeptide | ILNCECKRGFRRLKELVYMRCLGNSWSSNCQCTSNSHDKSRKQVTAQ |
| | Sequence | LEHQKEQQTTTDMQKPTQSMHQENLTGHCREPPPWKHEDSKRIYHFV |
| | | EGQSVHYECIPGYKALQRGPAISICKMKCGKTGWTQPQLTCVDEREH |
| | | HRFLASEESQGSRNSSPESETSCPITTTDFPQPTETTAMTETFVLTM |
| | | EYK |
| 106 | Construct E | APTSSSTKKTQLQLEHLLLDLQMILNGINNYKNPKLTRMLTFKFYMP |
| | polypeptide | KKATELKHLQCLEEELKPLEEVLNLAQSKNFHLRPRDLISNINVIVL |
| | sequence: | ELKGSETTFMCEYADETATIVEFLNRWITFSQSIISTLTSGGGGSGG |
| | HuIL2(C125S)- | GGGPLGVRGGGGGSGGGGSELCDDDPPEIPHATFKAMAYKEGTMLNC |
| | 2x(SG4)- | ECKRGFRRIKSGSLYMLCTGNSSHSSWDNQCQCTSSATRNTTKQVTP |
| | MMPcs1- | QPEEQKERKTTEMQSPMQPVDQASLPGHCREPPPWENEATERIYHFV |
| | 2x(G4S)- | VGQMVYYQCVQGYRALHRGPAESVCKMTHGKTRWTQPQLICTGEMET |
| | IL2Ralpha- | SQFPGEEKPQASPEGRPESETSCLVTTTDFQIQTEMAATMETSIFTT |
| | hu IgG1 Fc | EYQDKTHTCPPCPAPELLGGPSVFLFPPKPKDTLMISRTPEVTCVVV |
| | 6xHIS | DVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSFF |
| | | LYSKLTVDKSRWQQGNVFSCSVMHEALHNHYTQKSLSLSPGHHHHHH |
| 107 | Construct S | APTSSSTKKTQLQLEHLLLDLQMILNGINNYKNPKLTRMLTFKFYMP |
| | polypeptide | KKATELKHLQCLEEELKPLEEVLNLAQSKNFHLRPRDLISNINVIVL |
| | sequence: | ELKGSETTFMCEYADETATIVEFLNRWITFCQSIISTLTSGGGGSGG |
| | hIL2- | GGGPLGVRGGGGGSGGGGSELCDDDPPEIPHATFKAMAYKEGTMLNC |
| | 2x(SG4)- | ECKRGFRRIKSGSLYMLCTGNSSHSSWDNQCQCTSSATRNTTKQVTP |
| | MMPcs1- | QPEEQKERKTTEMQSPMQPVDQASLPGHCREPPPWENEATERIYHFV |
| | 2x(G4S)- | VGQMVYYQCVQGYRALHRGPAESVCKMTHGKTRWTQPQLICTGEMET |
| | hIL2Ralpha- | SQFPGEEKPQASPEGRPESETSCLVTTTDFQIQTEMAATMETSIFTT |
| | hIgG1Fc_mut 1 | EYQDKTHTCPPCPAPELLGGPSVFLFPPKPKDTLMISRTPEVTCVVV |
| | (K392D; | DVSHEDPEVKFNWYVDGVEVHNAKTKPREEQYNSTYRVVSVLTVLHQ |
| | K409D) | DWLNGKEYKCKVSNKALPAPIEKTISKAKGQPREPQVYTLPPSRDEL |
| | | TKNQVSLTCLVKGFYPSDIAVEWESNGQPENNYDTTPPVLDSDGSFF |
| | | LYSDLTVDKSRWQQGNVFSCSVMHEALHNHYTQKSLSLSPGHHHHHH |
| 108 | Construct T | DKTHTCPPCPAPELLGGPSVFLFPPKPKDTLMISRTPEVTCVVVDVS |
| | polypeptide | HEDPEVKFNWYVDGVEVHNAKTKPREEQYNSTYRVVSVLTVLHQDWL |
| | sequence | NGKEYKCKVSNKALPAPIEKTISKAKGQPREPQVYTLPPSRKELTKN |
| | including | QVSLTCLVKGFYPSDIAVEWESNGQPENNYKTTPPVLKSDGSFFLYS |
| | hIgG1 | KLTVDKSRWQQGNVFSCSVMHEALHNHYTQKSLSLSPGHHHHHH |
| | Fc_mut 2 | |
| | (D356K, | |
| | D399K) | |
| 109 | hu IgG1 Fc- | DKTHTCPPCPAPELLGGPSVFLFPPKPKDTLMISRTPEVTCVVVDVS |
| | Hu | HEDPEVKFNWYVDGVEVHNAKTKPREEQYNSTYRVVSVLTVLHQDWL |
| | IL2(C125S)- | NGKEYKCKVSNKALPAPIEKTISKAKGQPREPQVYTLPPSRDELTKN |
| | 2x(SG4)- | QVSLTCLVKGFYPSDIAVEWESNGQPENNYKTTPPVLDSDGSFFLYS |
| | MMPcs1- | KLTVDKSRWQQGNVFSCSVMHEALHNHYTQKSLSLSPGAPTSSSTKK |
| | 2x(G4S)- | TQLQLEHLLLDLQMILNGINNYKNPKLTRMLTFKFYMPKKATELKHL |
| | IL2Ralpha | QCLEEELKPLEEVLNLAQSKNFHLRPRDLISNINVIVLELKGSETTF |
| | Polypeptide | MCEYADETATIVEFLNRWITFSQSIISTLTSGGGGSGGGGGPLGVRG |
| | Sequence | GGGGSGGGGSELCDDDPPEIPHATFKAMAYKEGTMLNCECKRGFRRI |
| | | KSGSLYMLCTGNSSHSSWDNQCQCTSSATRNTTKQVTPQPEEQKERK |
| | | TTEMQSPMQPVDQASLPGHCREPPPWENEATERIYHFVVGQMVYYQC |
| | | VQGYRALHRGPAESVCKMTHGKTRWTQPQLICTGEMETSQFPGEEKP |
| | | QASPEGRPESETSCLVTTTDFQIQTEMAATMETSIFTTEYQ |
| 110 | hIL2- | APTSSSTKKTQLQLEHLLLDLQMILNGINNYKNPKLTRMLTFKFYMP |
| | 2x(SG4)- | KKATELKHLQCLEEELKPLEEVLNLAQSKNFHLRPRDLISNINVIVL |
| | MMPcs1- | ELKGSETTFMCEYADETATIVEFLNRWITFCQSIISTLTSGGGGSGG |
| | 2x(G4S)- | GGGPLGVRGGGGGSGGGGSAVNGTSQFTCFYNSRANISCVWSQDGAL |
| | hIL2Rbeta- | QDTSCQVHAWPDRRRWNQTCELLPVSQASWACNLILGAPDSQKLTTV |
| | hIgG1Fc | DIVTLRVLCREGVRWRVMAIQDFKPFENLRLMAPISLQVVHVETHRC |
| | (Construct U) | NISWEISQASHYFERHLEFEARTLSPGHTWEEAPLLTLKQKQEWICL |
| | polypeptide | ETLTPDTQYEFQVRVKPLQGEFTTWSPWSQPLAFRTKPAALGKDTDK |
| | Sequence | THTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNG |
| | | KEYKCKVSNKALPAPIEKTISKAKGQPREPQVYTLPPSRDELTKNQV |
| | | SLTCLVKGFYPSDIAVEWESNGQPENNYKTTPPVLDSDGSFFLYSKL |
| | | TVDKSRWQQGNVFSCSVMHEALHNHYTQKSLSLSPG |
| 111 | hIL2- | APTSSSTKKTQLQLEHLLLDLQMILNGINNYKNPKLTRMLTFKFYMP |
| | 2x(SG4)- | KKATELKHLQCLEEELKPLEEVLNLAQSKNFHLRPRDLISNINVIVL |
| | MMPcs1- | ELKGSETTFMCEYADETATIVEFLNRWITFCQSIISTLTSGGGGSGG |
| | 2x(G4S)- | GGGPLGVRGGGGGSGGGGSLNTTILTPNGNEDTTADFFLTTMPTDSL |
| | hIL2Rgamma- | SVSTLPLPEVQCFVFNVEYMNCTWNSSSEPQPTNLTLHYWYKNSDND |
| | hIgG1Fc | KVQKCSHYLFSEEITSGCQLQKKEIHLYQTFVVQLQDPREPRRQATQ |
| | polypeptide | MLKLQNLVIPWAPENLTLHKLSESQLELNWNNRFLNHCLEHLVQYRT |
| | sequence | DWDHSWTEQSVDYRHKFSLPSVDGQKRYTFRVRSRFNPLCGSAQHWS |
| | | EWSHPIHWGSNTSKENPFLFALEADKTHTCPPCPAPELLGGPSVFLF |
| | | PPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTIS |
| | | KAKGQPREPQVYTLPPSRDELTKNQVSLTCLVKGFYPSDIAVEWESN |
| | | GQPENNYKTTPPVLDSDGSFELYSKLTVDKSRWQQGNVFSCSVMHEA |
| | | LHNHYTQKSLSLSPG |
| 111- | Not Used | |
| 119 | | |

Other

| | | |
|---|---|---|
| 120 | Gly-Ser rich | SGGGGSGGGG |
| | linker | |
| | polypeptide | |
| | sequence | |
| 121- | Not Used | |
| 129 | | |

Fusion polypeptides (DNA coding sequences)

| | | |
|---|---|---|
| 130 | Construct A | ATGGGTTGGTCCTGCATCATCCTGTTCCTGGTCGCCACCGCCACTGG |
| | DNA | GGTCCACTCCGCACCTACATCATCATCAACTTCATCCTCCACCGCTG |
| | sequence | AGGCTCAGCAACAACAGCAACAACAGCAGCAGCAGCAGCAGCATCTG |
| | (mIL2- | GAGCAGCTGCTGATGGACCTGCAGGAGCTGCTGTCCAGAATGGAGAA |
| | 2x(SG4)- | CTACCGCAATCTGAAGCTGCCAAGGATGCTGACCTTCAAGTTTTATC |
| | MMPcs1- | TGCCCAAGCAGGCCACAGAGCTGAAGGACCTGCAGTGCCTGGAGGAT |
| | 2x(G4S)- | GAGCTGGGCCCACTGAGGCACGTGCTGGACCTGACCCAGAGCAAGTC |
| | IL2Ralpha- | TTTCCAGCTGGAGGATGCTGAGAACTTTATCTCCAATATCCGGGTGA |
| | 6His) | CCGTGGTGAAGCTGAAGGGCAGCGACAACACATTCGAGTGCCAGTTT |
| | | GACGATGAGTCTGCCACCGTGGTGGATTTCCTGAGGCGGTGGATCGC |
| | | TTTTTGTCAGAGCATCATCTCCACAAGCCCTCAGTCTGGAGGAGGTG |
| | | GCAGCGGAGGAGGAGGTGGCCCACTGGGCGTGAGGGGTGGCGGCGGC |
| | | GGCTCTGGCGGCGGCGGCTCCGAGCTGTGCCTGTACGACCCCCCTGA |
| | | GGTGCCCAATGCCACCTTCAAGGCTCTGTCTTATAAGAACGGCACAA |
| | | TCCTGAATTGCGAGTGTAAGAGGGGCTTTAGACGCCTGAAGGAGCTG |
| | | GTGTACATGCGGTGTCTGGGCAACTCCTGGTCCAGCAATTGCCAGTG |
| | | TACCTCTAACTCCCATGACAAGAGCAGAAAGCAGGTGACAGCCCAGC |
| | | TGGAGCACCAGAAGGAGCAGCAGACCACAACCGATATGCAGAAGCCC |
| | | ACCCAGTCTATGCACCAGGAGAATCTGACAGGCCATTGCAGAGAGCC |
| | | ACCCCCTTGGAAGCACGAGGATAGCAAGCGCATCTATCATTTCGTGG |
| | | AGGGCCAGTCTGTGCACTACGAGTGTATCCCCGGCTATAAGGCCCTG |
| | | CAGAGAGGCCCTGCTATCTCCATCTGCAAGATGAAGTGTGGCAAGAC |
| | | CGGCTGGACACAGCCTCAGCTGACCTGCGTGGACGAGAGGGAGCACC |
| | | ATCGGTTCCTGGCTAGCGAGGAGTCTCAGGGCTCCCGCAACTCTTCC |
| | | CCTGAGAGCGAGACATCTTGTCCAATCACAACCACAGATTTTCCACA |
| | | GCCCACCGAGACAACCGCTATGACAGAGACCTTCGTGCTGACTATGG |
| | | AATACAAACACCACCACCACCACCACTAATGA |
| 131 | Construct B | ATGGGTTGGTCCTGCATCATCCTGTTCCTGGTCGCCACCGCCACTGG |
| | DNA | GGTCCACTCCGCACCTACATCATCATCAACTTCATCCTCCACCGCTG |
| | sequence: m | AGGCTCAGCAACAACAGCAACAACAGCAGCAGCAGCAGCAGCATCTG |
| | IL2-2x(SG4)- | GAGCAGCTGCTGATGGACCTGCAGGAGCTGCTGTCCAGAATGGAGAA |
| | MMPcs1- | CTACCGCAATCTGAAGCTGCCAAGGATGCTGACCTTCAAGTTTTATC |
| | 2x(G4S)- | TGCCCAAGCAGGCCACAGAGCTGAAGGACCTGCAGTGCCTGGAGGAT |
| | IL2Ralpha- | GAGCTGGGCCCACTGAGGCACGTGCTGGACCTGACCCAGAGCAAGTC |
| | mIgG1 Fc | TTTCCAGCTGGAGGATGCTGAGAACTTTATCTCCAATATCCGGGTGA |
| | | CCGTGGTGAAGCTGAAGGGCAGCGACAACACATTCGAGTGCCAGTTT |
| | | GACGATGAGTCTGCCACCGTGGTGGATTTCCTGAGGCGGTGGATCGC |
| | | TTTTTGTCAGAGCATCATCTCCACAAGCCCTCAGTCTGGAGGAGGTG |
| | | GCAGCGGAGGAGGAGGTGGCCCACTGGGCGTGAGGGGTGGCGGCGGC |
| | | GGCTCTGGCGGCGGCGGCTCCGAGCTGTGCCTGTACGACCCCCCTGA |
| | | GGTGCCCAATGCCACCTTCAAGGCTCTGTCTTATAAGAACGGCACAA |
| | | TCCTGAATTGCGAGTGTAAGAGGGGCTTTAGACGCCTGAAGGAGCTG |
| | | GTGTACATGCGGTGTCTGGGCAACTCCTGGTCCAGCAATTGCCAGTG |
| | | TACCTCTAACTCCCATGACAAGAGCAGAAAGCAGGTGACAGCCCAGC |
| | | TGGAGCACCAGAAGGAGCAGCAGACCACAACCGATATGCAGAAGCCC |
| | | ACCCAGTCTATGCACCAGGAGAATCTGACAGGCCATTGCAGAGAGCC |
| | | ACCCCCTTGGAAGCACGAGGATAGCAAGCGCATCTATCATTTCGTGG |
| | | AGGGCCAGTCTGTGCACTACGAGTGTATCCCCGGCTATAAGGCCCTG |
| | | CAGAGAGGCCCTGCTATCTCCATCTGCAAGATGAAGTGTGGCAAGAC |
| | | CGGCTGGACACAGCCTCAGCTGACCTGCGTGGACGAGAGGGAGCACC |
| | | ATCGGTTCCTGGCTAGCGAGGAGTCTCAGGGCTCCCGCAACTCTTCC |
| | | CCTGAGAGCGAGACATCTTGTCCAATCACAACCACAGATTTTCCACA |
| | | GCCCACCGAGACAACCGCTATGACAGAGACCTTCGTGCTGACTATGG |
| | | AATACAAAGGATGCAAACCCTGTATCTGTACCGTGCCCGAGGTCTCT |
| | | TCCGTCTTTATTTTCCCCCCCAAGCCTAAGGATGTGCTGACTATTAC |
| | | TCTGACCCCCAAGGTGACATGCGTGGTGGTGGACATCAGCAAGGACG |
| | | ATCCTGAGGTGCAGTTCTCTTGGTTTGTGGACGATGTGGAGGTGCAC |
| | | ACCGCCCAGACACAGCCAAGGGAGGAGCAGTTCAATAGCACCTTTCG |
| | | GTCCGTGAGCGAGCTGCCCATCATGCATCAGGATTGGCTGAATGGCA |
| | | AGGAGTTCAAGTGCAGAGTGAACTCTGCCGCTTTTCCCGCTCCTATC |
| | | GAGAAGACCATCTCCAAGACAAAGGGCCGCCCAAAGGCTCCACAGGT |
| | | GTACACCATCCCACCTCCAAAGGAGCAGATGGCTAAGGACAAGGTGT |
| | | CTCTGACCTGTATGATCACAGACTTCTTTCCTGAGGACATCACAGTG |
| | | GAGTGGCAGTGGAACGGCCAGCCTGCCGAGAACTATAAGAATACCCA |
| | | GCCAATCATGGACACAGATGGCTCTTACTTCGTGTATTCCAAGCTGA |
| | | ACGTGCAGAAGTCCAATTGGGAGGCTGGCAACACCTTTACATGTAGC |
| | | GTGCTGCACGAAGGTCTGCATAACCATCATACCGAAAAATCACTGTC |
| | | ACACTCCCCTGGAAAATAATGA |
| 132 | Construct C | ATGGGTTGGTCCTGCATCATCCTGTTCCTGGTCGCCACCGCCACTGG |
| | DNA | GGTCCACTCCGCACCTACATCATCATCAACTTCATCCTCCACCGCTG |
| | sequence: m | AGGCTCAGCAACAACAGCAACAACAGCAGCAGCAGCAGCAGCATCTG |
| | IL2(C140S)- | GAGCAGCTGCTGATGGACCTGCAGGAGCTGCTGTCCAGAATGGAGAA |
| | 2x(SG4)- | CTACCGCAATCTGAAGCTGCCAAGGATGCTGACCTTCAAGTTTTATC |
| | MMPcs1- | TGCCCAAGCAGGCCACAGAGCTGAAGGACCTGCAGTGCCTGGAGGAT |
| | 2x(G4S)- | GAGCTGGGCCCACTGAGGCACGTGCTGGACCTGACCCAGAGCAAGTC |
| | IL2Ralpha- | TTTCCAGCTGGAGGATGCTGAGAACTTTATCTCCAATATCCGGGTGA |
| | mIgG1 | CCGTGGTGAAGCTGAAGGGCAGCGACAACACATTCGAGTGCCAGTTT |
| | Fc(T252M)- | GACGATGAGTCTGCCACCGTGGTGGATTTCCTGAGGCGGTGGATCGC |
| | 6xHIS | TTTTTCCCAGAGCATCATCTCCACAAGCCCTCAGTCTGGAGGAGGTG |
| | | GCAGCGGAGGAGGAGGTGGCCCACTGGGCGTGAGGGGTGGCGGCGGC |
| | | GGCTCTGGCGGCGGCGGCTCCGAGCTGTGCCTGTACGACCCCCCTGA |
| | | GGTGCCCAATGCCACCTTCAAGGCTCTGTCTTATAAGAACGGCACAA |
| | | TCCTGAATTGCGAGTGTAAGAGGGGCTTTAGACGCCTGAAGGAGCTG |
| | | GTGTACATGCGGTGTCTGGGCAACTCCTGGTCCAGCAATTGCCAGTG |
| | | TACCTCTAACTCCCATGACAAGAGCAGAAAGCAGGTGACAGCCCAGC |
| | | TGGAGCACCAGAAGGAGCAGCAGACCACAACCGATATGCAGAAGCCC |
| | | ACCCAGTCTATGCACCAGGAGAATCTGACAGGCCATTGCAGAGAGCC |
| | | ACCCCCTTGGAAGCACGAGGATAGCAAGCGCATCTATCATTTCGTGG |
| | | AGGGCCAGTCTGTGCACTACGAGTGTATCCCCGGCTATAAGGCCCTG |
| | | CAGAGAGGCCCTGCTATCTCCATCTGCAAGATGAAGTGTGGCAAGAC |
| | | CGGCTGGACACAGCCTCAGCTGACCTGCGTGGACGAGAGGGAGCACC |
| | | ATCGGTTCCTGGCTAGCGAGGAGTCTCAGGGCTCCCGCAACTCTTCC |
| | | CCTGAGAGCGAGACATCTTGTCCAATCACAACCACAGATTTTCCACA |
| | | GCCCACCGAGACAACCGCTATGACAGAGACCTTCGTGCTGACTATGG |
| | | AATACAAAGGATGCAAACCCTGTATCTGTACCGTGCCCGAGGTCTCT |
| | | TCCGTCTTTATTTTCCCCCCCAAGCCTAAGGATGTGCTGATGATTAC |
| | | TCTGACCCCCAAGGTGACATGCGTGGTGGTGGACATCAGCAAGGACG |
| | | ATCCTGAGGTGCAGTTCTCTTGGTTTGTGGACGATGTGGAGGTGCAC |
| | | ACCGCCCAGACACAGCCAAGGGAGGAGCAGTTCAATAGCACCTTTCG |
| | | GTCCGTGAGCGAGCTGCCCATCATGCATCAGGATTGGCTGAATGGCA |
| | | AGGAGTTCAAGTGCAGAGTGAACTCTGCCGCTTTTCCCGCTCCTATC |
| | | GAGAAGACCATCTCCAAGACAAAGGGCCGCCCAAAGGCTCCACAGGT |
| | | GTACACCATCCCACCTCCAAAGGAGCAGATGGCTAAGGACAAGGTGT |
| | | CTCTGACCTGTATGATCACAGACTTCTTTCCTGAGGACATCACAGTG |
| | | GAGTGGCAGTGGAACGGCCAGCCTGCCGAGAACTATAAGAATACCCA |
| | | GCCAATCATGGACACAGATGGCTCTTACTTCGTGTATTCCAAGCTGA |
| | | ACGTGCAGAAGTCCAATTGGGAGGCTGGCAACACCTTTACATGTAGC |
| | | GTGCTGCACGAAGGTCTGCATAACCATCATACCGAAAAATCACTGTC |
| | | ACACTCCCCTGGACACCACCACCACCACCACTAATGA |
| 133 | Construct R | ATGGGTTGGTCCTGCATCATCCTGTTCCTGGTCGCCACCGCCACTGG |
| | DNA | GGTCCACTCCGCACCTACATCATCATCAACTTCATCCTCCACCGCTG |
| | sequence: | AGGCTCAGCAACAACAGCAACAACAGCAGCAGCAGCAGCAGCATCTG |
| | mIL2(C140S)- | GAGCAGCTGCTGATGGACCTGCAGGAGCTGCTGTCCAGAATGGAGAA |
| | 2x(SG4)- | CTACCGCAATCTGAAGCTGCCAAGGATGCTGACCTTCAAGTTTTATC |
| | MMPcs1- | TGCCCAAGCAGGCCACAGAGCTGAAGGACCTGCAGTGCCTGGAGGAT |
| | 2x(G4S)- | GAGCTGGGCCCACTGAGGCACGTGCTGGACCTGACCCAGAGCAAGTC |
| | IL2Ralpha- | TTTCCAGCTGGAGGATGCTGAGAACTTTATCTCCAATATCCGGGTGA |
| | hu IgG1 Fc- | CCGTGGTGAAGCTGAAGGGCAGCGACAACACATTCGAGTGCCAGTTT |
| | 6xHIS | GACGATGAGTCTGCCACCGTGGTGGATTTCCTGAGGCGGTGGATCGC |
| | | TTTTTCCCAGAGCATCATCTCCACAAGCCCTCAGTCTGGAGGAGGTG |
| | | GCAGCGGAGGAGGAGGTGGCCCACTGGGCGTGAGGGGTGGCGGCGGC |
| | | GGCTCTGGCGGCGGCGGCTCCGAGCTGTGCCTGTACGACCCCCCTGA |
| | | GGTGCCCAATGCCACCTTCAAGGCTCTGTCTTATAAGAACGGCACAA |
| | | TCCTGAATTGCGAGTGTAAGAGGGGCTTTAGACGCCTGAAGGAGCTG |
| | | GTGTACATGCGGTGTCTGGGCAACTCCTGGTCCAGCAATTGCCAGTG |
| | | TACCTCTAACTCCCATGACAAGAGCAGAAAGCAGGTGACAGCCCAGC |
| | | TGGAGCACCAGAAGGAGCAGCAGACCACAACCGATATGCAGAAGCCC |
| | | ACCCAGTCTATGCACCAGGAGAATCTGACAGGCCATTGCAGAGAGCC |
| | | ACCCCCTTGGAAGCACGAGGATAGCAAGCGCATCTATCATTTCGTGG |
| | | AGGGCCAGTCTGTGCACTACGAGTGTATCCCCGGCTATAAGGCCCTG |
| | | CAGAGAGGCCCTGCTATCTCCATCTGCAAGATGAAGTGTGGCAAGAC |
| | | CGGCTGGACACAGCCTCAGCTGACCTGCGTGGACGAGAGGGAGCACC |
| | | ATCGGTTCCTGGCTAGCGAGGAGTCTCAGGGCTCCCGCAACTCTTCC |
| | | CCTGAGAGCGAGACATCTTGTCCAATCACAACCACAGATTTTCCACA |
| | | GCCCACCGAGACAACCGCTATGACAGAGACCTTCGTGCTGACTATGG |
| | | AATACAAAGATAAGACTCATACCTGTCCACCCTGTCCTGCTCCTGAA |
| | | CTGCTGGGCGGTCCTTCCGTGTTCCTGTTCCCTCCAAAACCTAAAGA |
| | | TACCCTGATGATCTCCAGGACCCCTGAGGTGACATGCGTGGTGGTGG |
| | | ACGTGAGCCACGAGGACCCCGAGGTGAAGTTCAACTGGTACGTGGAC |
| | | GGCGTGGAGGTGCATAATGCCAAGACAAAGCCCAGGGAGGAGCAGTA |
| | | CAACAGCACCTATCGGGTGGTGTCTGTGCTGACAGTGCTGCACCAGG |
| | | ATTGGCTGAACGGCAAGGAGTATAAGTGCAAGGTGTCTAATAAGGCC |
| | | CTGCCTGCTCCAATCGAGAAGACCATCTCCAAGGCCAAGGGCCAGCC |
| | | CAGAGAGCCTCAGGTGTACACACTGCCCCCTAGCCGCGACGAGCTGA |
| | | CCAAGAACCAGGTGTCTCTGACATGTCTGGTGAAGGGCTTCTATCCA |
| | | TCTGACATCGCTGTGGAGTGGGAGTCCAATGGCCAGCCCGAGAACAA |
| | | TTACAAGACCACACCACCCGTGCTGGACTCTGATGGCTCCTTCTTTC |
| | | TGTATTCCAAGCTGACCGTGGACAAGAGCAGATGGCAGCAGGGCAAC |
| | | GTGTTCTCCTGTAGCGTGATGCACGAAGCCCTGCACAACCATTACAC |
| | | TCAGAAAAGCCTGTCCCTGTCCCCTGGGCACCACCACCACCACCACT |
| | | AATGA |
| 134 | Construct D | ATGGGTTGGTCCTGCATCATCCTGTTCCTGGTCGCCACCGCCACTGG |
| | DNA | GGTCCACTCCGCACCTACATCATCATCAACTTCATCCTCCACCGCTG |
| | Sequence | AGGCTCAGCAACAACAGCAACAACAGCAGCAGCAGCAGCAGCATCTG |
| | mIL2(C140S)- | GAGCAGCTGCTGATGGACCTGCAGGAGCTGCTGTCCAGAATGGAGAA |
| | 2x(SG4)- | CTACCGCAATCTGAAGCTGCCAAGGATGCTGACCTTCAAGTTTTATC |
| | MMPcs1- | TGCCCAAGCAGGCCACAGAGCTGAAGGACCTGCAGTGCCTGGAGGAT |
| | 2x(G4S)- | GAGCTGGGCCCACTGAGGCACGTGCTGGACCTGACCCAGAGCAAGTC |
| | sIL2Ralpha- | TTTCCAGCTGGAGGATGCTGAGAACTTTATCTCCAATATCCGGGTGA |
| | mIgG1 | CCGTGGTGAAGCTGAAGGGCAGCGACAACACATTCGAGTGCCAGTTT |
| | Fc(T252M)- | GACGATGAGTCTGCCACCGTGGTGGATTTCCTGAGGCGGTGGATCGC |
| | 6xHIS | TTTTTCCCAGAGCATCATCTCCACAAGCCCTCAGTCTGGAGGAGGTG |
| | | GCAGCGGAGGAGGAGGTGGCCCACTGGGCGTGAGGGGTGGCGGCGGC |
| | | GGCTCTGGCGGCGGCGGCTCCGAGCTGTGCCTGTACGACCCCCCTGA |
| | | GGTGCCCAATGCCACCTTCAAGGCTCTGTCTTATAAGAACGGCACAA |
| | | TCCTGAATTGCGAGTGTAAGAGGGGCTTTAGACGCCTGAAGGAGCTG |
| | | GTGTACATGCGGTGTCTGGGCAACTCCTGGTCCAGCAATTGCCAGTG |
| | | TACCTCTAACTCCCATGACAAGAGCAGAAAGCAGGTGACAGCCCAGC |
| | | TGGAGCACCAGAAGGAGCAGCAGACCACAACCGATATGCAGAAGCCC |
| | | ACCCAGTCTATGCACCAGGAGAATCTGACAGGCCATTGCAGAGAGCC |
| | | ACCCCCTTGGAAGCACGAGGATAGCAAGCGCATCTATCATTTCGTGG |
| | | AGGGCCAGTCTGTGCACTACGAGTGTATCCCCGGCTATAAGGCCCTG |
| | | CAGAGAGGCCCTGCTATCTCCATCTGCAAGATGAAGTGTGGCAAGAC |
| | | CGGCTGGACACAGCCTCAGCTGACCTGCGTGGACGAGAGGGAGCACC |
| | | ATCGGTTCCTGGCTAGCGAGGAGTCTGGATGCAAACCCTGTATCTGT |
| | | ACCGTGCCCGAGGTCTCTTCCGTCTTTATTTTCCCCCCCAAGCCTAA |
| | | GGATGTGCTGATGATTACTCTGACCCCCAAGGTGACATGCGTGGTGG |
| | | TGGACATCAGCAAGGACGATCCTGAGGTGCAGTTCTCTTGGTTTGTG |
| | | GACGATGTGGAGGTGCACACCGCCCAGACACAGCCAAGGGAGGAGCA |
| | | GTTCAATAGCACCTTTCGGTCCGTGAGCGAGCTGCCCATCATGCATC |
| | | AGGATTGGCTGAATGGCAAGGAGTTCAAGTGCAGAGTGAACTCTGCC |
| | | GCTTTTCCCGCTCCTATCGAGAAGACCATCTCCAAGACAAAGGGCCG |
| | | CCCAAAGGCTCCACAGGTGTACACCATCCCACCTCCAAAGGAGCAGA |
| | | TGGCTAAGGACAAGGTGTCTCTGACCTGTATGATCACAGACTTCTTT |
| | | CCTGAGGACATCACAGTGGAGTGGCAGTGGAACGGCCAGCCTGCCGA |
| | | GAACTATAAGAATACCCAGCCAATCATGGACACAGATGGCTCTTACT |
| | | TCGTGTATTCCAAGCTGAACGTGCAGAAGTCCAATTGGGAGGCTGGC |
| | | AACACCTTTACATGTAGCGTGCTGCACGAAGGTCTGCATAACCATCA |
| | | TACCGAAAAATCACTGTCACACTCCCCTGGACACCACCACCACCACC |
| | | ACTAATGA |
| 135 | Construct E | ATGGGCTGGTCCTGCATCATTCTGTTTCTGGTGGCTACCGCCACCGG |
| | DNA | CGTGCACTCTGCTCCTACATCCTCCAGCACCAAGAAAACCCAGCTGC |
| | sequence: | AGTTGGAGCATCTGCTGCTGGACCTGCAGATGATCCTGAACGGCATC |
| | HuIL2(C125S)- | AACAACTACAAGAACCCCAAGCTGACCCGGATGCTGACCTTCAAGTT |
| | 2x(SG4)- | CTACATGCCCAAGAAGGCCACCGAGCTGAAACATCTGCAGTGCCTGG |
| | MMPcs1- | AAGAGGAACTGAAGCCCCTGGAAGAAGTGCTGAATCTGGCCCAGTCC |
| | 2x(G4S)- | AAGAACTTCCACCTGAGGCCTCGGGACCTGATCTCCAACATCAACGT |
| | IL2Ralpha- | GATCGTGCTCGAGCTGAAGGGCTCCGAGACAACCTTCATGTGCGAGT |
| | hu IgG1 Fc | ACGCCGACGAGACAGCTACCATCGTGGAATTTCTGAACCGGTGGATC |
| | 6xHIS | ACCTTCAGCCAGTCCATCATCAGCACCCTGACATCTGGCGGCGGAGG |
| | | ATCTGGCGGAGGCGGAGGACCTTTGGGAGTTCGCGGCGGTGGTGGTG |
| | | GCAGCGGAGGTGGTGGATCTGAGCTGTGTGACGACGACCCTCCTGAG |
| | | ATCCCTCACGCCACCTTTAAGGCCATGGCTTACAAAGAGGGCACCAT |
| | | GCTGAACTGCGAGTGCAAGAGAGGCTTCCGGCGGATCAAGTCCGGCA |
| | | GCCTGTATATGCTGTGCACCGGCAACTCCAGCCACTCCTCTTGGGAC |
| | | AACCAGTGCCAGTGCACCAGCTCTGCTACCCGGAACACCACCAAGCA |
| | | AGTGACCCCTCAGCCTGAGGAACAGAAAGAGCGCAAGACCACCGAGA |
| | | TGCAGAGCCCCATGCAGCCTGTGGATCAGGCTTCTCTGCCTGGCCAC |
| | | TGTAGAGAGCCTCCACCTTGGGAGAATGAGGCTACCGAGAGAATCTA |
| | | CCACTTCGTCGTGGGACAGATGGTGTACTACCAGTGCGTGCAGGGCT |
| | | ACCGCGCTCTGCATAGAGGACCAGCAGAGTCCGTGTGCAAGATGACC |
| | | CACGGCAAGACCAGATGGACCCAGCCTCAGCTGATCTGCACCGGCGA |
| | | GATGGAAACCTCTCAGTTCCCCGGCGAGGAAAAGCCTCAGGCCTCTC |
| | | CTGAAGGCAGACCCGAGTCTGAGACATCCTGTCTCGTGACCACCACA |
| | | GACTTCCAGATCCAGACCGAGATGGCCGCTACCATGGAAACCAGCAT |
| | | CTTCACCACCGAGTACCAGGACAAGACCCACACCTGTCCTCCATGTC |
| | | CTGCTCCAGAATTGCTCGGCGGACCCTCCGTGTTCCTGTTTCCTCCA |
| | | AAGCCTAAGGACACCCTGATGATCTCTCGGACCCCTGAAGTGACCTG |
| | | CGTGGTGGTCGATGTGTCTCACGAGGATCCCGAAGTGAAGTTCAATT |
| | | GGTACGTGGACGGCGTGGAAGTGCACAACGCCAAGACCAAGCCTAGA |
| | | GAGGAACAGTACAACTCCACCTACAGAGTGGTGTCCGTGCTGACCGT |
| | | GCTGCACCAGGATTGGCTGAATGGCAAAGAGTACAAGTGCAAGGTGT |
| | | CCAACAAGGCCCTGCCTGCTCCTATCGAAAAGACCATCTCCAAGGCC |
| | | AAGGGCCAGCCTAGGGAACCCCAGGTTTACACCTTGCCTCCATCTCG |
| | | GGACGAGCTGACCAAGAACCAGGTGTCCCTGACCTGTCTGGTCAAGG |
| | | GCTTCTACCCCTCCGATATCGCCGTGGAATGGGAGTCTAATGGCCAG |
| | | CCTGAAAACAATTACAAGACAACCCCTCCTGTGCTGGACTCCGACGG |
| | | CTCATTCTTCCTGTACAGCAAGCTGACAGTGGACAAGTCCAGATGGC |
| | | AGCAGGGCAACGTGTTCTCCTGCTCCGTGATGCATGAGGCCCTGCAC |
| | | AACCACTACACCCAGAAGTCCCTGTCTCTGTCCCCTGGCCACCATCA |
| | | CCATCATCACTGATAA |
| 136 | Construct S | ATGGGTTGGTCCTGCATCATCCTGTTCCTGGTCGCCACCGCCACTGG |
| | DNA | GGTCCACTCCGCACCTACTTCAAGTTCTACAAAGAAAACACAGCTAC |
| | sequence: | AACTGGAGCATTTACTTCTGGATTTACAGATGATTTTGAATGGAATT |
| | hIL2- | AATAATTACAAGAATCCCAAACTCACCAGGATGCTCACATTTAAGTT |
| | 2x(SG4)- | TTACATGCCCAAGAAGGCCACAGAACTGAAACATCTTCAGTGTCTAG |
| | MMPcs1- | AAGAAGAACTCAAACCTCTGGAGGAAGTGCTAAATTTAGCTCAAAGC |
| | 2x(G4S)- | AAAAACTTTCACTTAAGACCCAGGGACTTAATCAGCAATATCAACGT |
| | hIL2Ralpha- | AATAGTTCTGGAACTAAAGGGATCTGAAACAACATTCATGTGTGAAT |
| | hIgG1Fc_mut 1 | ATGCTGATGAGACAGCAACCATTGTAGAATTTCTGAACAGATGGATT |
| | (K392D; | ACCTTTTGTCAAAGCATCATCTCAACACTGACTTCTGGTGGCGGTGG |
| | K409D) | CTCTGGTGGCGGTGGCGGTCCTCTGGGTGTCAGAGGTGGTGGCGGTG |
| | | GCTCTGGTGGCGGTGGCTCTGAGCTCTGTGACGATGACCCGCCAGAG |
| | | ATCCCACACGCCACATTCAAAGCCATGGCCTACAAGGAAGGAACCAT |
| | | GTTGAACTGTGAATGCAAGAGAGGTTTCCGCAGAATAAAAAGCGGGT |
| | | CACTCTATATGCTCTGTACAGGAAACTCTAGCCACTCGTCCTGGGAC |
| | | AACCAATGTCAATGCACAAGCTCTGCCACTCGGAACACAACGAAACA |
| | | AGTGACACCTCAACCTGAAGAACAGAAAGAAAGGAAAACCACAGAAA |
| | | TGCAAAGTCCAATGCAGCCAGTGGACCAAGCGAGCCTTCCAGGTCAC |
| | | TGCAGGGAACCTCCACCATGGGAAAATGAAGCCACAGAGAGAATTTA |
| | | TCATTTCGTGGTGGGGCAGATGGTTTATTATCAGTGCGTCCAGGGAT |
| | | ACAGGGCTCTACACAGAGGTCCTGCTGAGAGCGTCTGCAAAATGACC |
| | | CACGGGAAGACAAGGTGGACCCAGCCCCAGCTCATATGCACAGGTGA |
| | | AATGGAGACCAGTCAGTTTCCAGGTGAAGAGAAGCCTCAGGCAAGCC |
| | | CCGAAGGCCGTCCTGAGAGTGAGACTTCCTGCCTCGTCACAACAACA |
| | | GATTTTCAAATACAGACAGAAATGGCTGCAACCATGGAGACGTCCAT |
| | | ATTTACAACAGAGTACCAGGACAAAACTCACACATGCCCACCGTGCC |
| | | CAGCACCTGAACTCCTGGGGGGACCGTCAGTCTTCCTCTTCCCCCCA |
| | | AAACCCAAGGACACCCTCATGATCTCCCGGACCCCTGAGGTCACATG |
| | | CGTGGTGGTGGACGTGAGCCACGAAGACCCTGAGGTCAAGTTCAACT |
| | | GGTACGTGGACGGCGTGGAGGTGCATAATGCCAAGACAAAGCCGCGG |
| | | GAGGAGCAGTACAACAGCACGTACCGTGTGGTCAGCGTCCTCACCGT |
| | | CCTGCACCAGGACTGGCTGAATGGCAAGGAGTACAAGTGCAAGGTCT |
| | | CCAACAAAGCCCTCCCAGCCCCCATCGAGAAAACCATCTCCAAAGCC |
| | | AAAGGGCAGCCCCGAGAACCACAGGTGTACACCCTGCCCCCATCCCG |
| | | GGATGAGCTGACCAAGAACCAGGTCAGCCTGACCTGCCTGGTCAAAG |
| | | GCTTCTATCCCAGCGACATCGCCGTGGAGTGGGAGAGCAATGGGCAG |
| | | CCGGAGAACAACTACGACACCACGCCTCCCGTGCTGGACTCCGACGG |
| | | CTCCTTCTTCCTCTATAGCGACCTCACCGTGGACAAGAGCAGGTGGC |
| | | AGCAGGGGAACGTCTTCTCATGCTCCGTGATGCATGAGGCTCTGCAC |
| | | AACCACTACACGCAGAAGAGCCTCTCCCTGTCTCCGGGTCACCACCA |
| | | CCACCACCACTAATGA |
| 137 | Construct T | ATGGGTTGGTCCTGCATCATCCTGTTCCTGGTCGCCACCGCCACTGG |
| | DNA | GGTCCACTCCGACAAAACTCACACATGCCCACCGTGCCCAGCACCTG |
| | sequence | AACTCCTGGGGGGACCGTCAGTCTTCCTCTTCCCCCCAAAACCCAAG |
| | including | GACACCCTCATGATCTCCCGGACCCCTGAGGTCACATGCGTGGTGGT |
| | hIgG1 | GGACGTGAGCCACGAAGACCCTGAGGTCAAGTTCAACTGGTACGTGG |
| | Fc_mut 2 | ACGGCGTGGAGGTGCATAATGCCAAGACAAAGCCGCGGGAGGAGCAG |
| | (D356K, | TACAACAGCACGTACCGTGTGGTCAGCGTCCTCACCGTCCTGCACCA |
| | D399K) | GGACTGGCTGAATGGCAAGGAGTACAAGTGCAAGGTCTCCAACAAAG |
| | | CCCTCCCAGCCCCCATCGAGAAAACCATCTCCAAAGCCAAAGGGCAG |
| | | CCCCGAGAACCACAGGTGTACACCCTGCCCCCATCCCGGAAAGAGCT |
| | | GACCAAGAACCAGGTCAGCCTGACCTGCCTGGTCAAAGGCTTCTATC |
| | | CCAGCGACATCGCCGTGGAGTGGGAGAGCAATGGGCAGCCGGAGAAC |
| | | AACTACAAGACCACGCCTCCCGTGCTGAAATCCGACGGCTCCTTCTT |
| | | CCTCTATAGCAAGCTCACCGTGGACAAGAGCAGGTGGCAGCAGGGGA |
| | | ACGTCTTCTCATGCTCCGTGATGCATGAGGCTCTGCACAACCACTAC |
| | | ACGCAGAAGAGCCTCTCCCTGTCTCCGGGTCACCACCACCACCACCA |
| | | CTAATGA |
| 138 | mIgG1 Fc- | ATGGGTTGGTCCTGCATCATCCTGTTCCTGGTCGCCACCGCCACTGG |
| | Murine IL2- | GGTCCACTCCGGATGCAAACCCTGTATCTGTACCGTGCCCGAGGTCT |
| | 2x(SG4)- | CTTCCGTCTTTATTTTCCCCCCCAAGCCTAAGGATGTGCTGACTATT |
| | MMPcs1-2x | ACTCTGACCCCCAAGGTGACATGCGTGGTGGTGGACATCAGCAAGGA |
| | (G4S)- | CGATCCTGAGGTGCAGTTCTCTTGGTTTGTGGACGATGTGGAGGTGC |
| | IL2Ralpha | ACACCGCCCAGACACAGCCAAGGGAGGAGCAGTTCAATAGCACCTTT |
| | (long kinetic | CGGTCCGTGAGCGAGCTGCCCATCATGCATCAGGATTGGCTGAATGG |
| | IL2 post | CAAGGAGTTCAAGTGCAGAGTGAACTCTGCCGCTTTTCCCGCTCCTA |
| | cleavage) | TCGAGAAGACCATCTCCAAGACAAAGGGCCGCCCAAAGGCTCCACAG |
| | DNA | GTGTACACCATCCCACCTCCAAAGGAGCAGATGGCTAAGGACAAGGT |
| | Sequence | GTCTCTGACCTGTATGATCACAGACTTCTTTCCTGAGGACATCACAG |
| | | TGGAGTGGCAGTGGAACGGCCAGCCTGCCGAGAACTATAAGAATACC |
| | | CAGCCAATCATGGACACAGATGGCTCTTACTTCGTGTATTCCAAGCT |
| | | GAACGTGCAGAAGTCCAATTGGGAGGCTGGCAACACCTTTACATGTA |
| | | GCGTGCTGCACGAAGGTCTGCATAACCATCATACCGAAAAATCACTG |
| | | TCACACTCCCCTGGAAAAGCACCTACATCATCATCAACTTCATCCTC |
| | | CACCGCTGAGGCTCAGCAACAACAGCAACAACAGCAGCAGCAGCAGC |
| | | AGCATCTGGAGCAGCTGCTGATGGACCTGCAGGAGCTGCTGTCCAGA |
| | | ATGGAGAACTACCGCAATCTGAAGCTGCCAAGGATGCTGACCTTCAA |
| | | GTTTTATCTGCCCAAGCAGGCCACAGAGCTGAAGGACCTGCAGTGCC |
| | | TGGAGGATGAGCTGGGCCCACTGAGGCACGTGCTGGACCTGACCCAG |
| | | AGCAAGTCTTTCCAGCTGGAGGATGCTGAGAACTTTATCTCCAATAT |
| | | CCGGGTGACCGTGGTGAAGCTGAAGGGCAGCGACAACACATTCGAGT |
| | | GCCAGTTTGACGATGAGTCTGCCACCGTGGTGGATTTCCTGAGGCGG |
| | | TGGATCGCTTTTTGTCAGAGCATCATCTCCACAAGCCCTCAGTCTGG |
| | | AGGAGGTGGCAGCGGAGGAGGAGGTGGCCCACTGGGCGTGAGGGGTG |
| | | GCGGCGGCGGCTCTGGCGGCGGCGGCTCCGAGCTGTGCCTGTACGAC |
| | | CCCCCTGAGGTGCCCAATGCCACCTTCAAGGCTCTGTCTTATAAGAA |
| | | CGGCACAATCCTGAATTGCGAGTGTAAGAGGGGCTTTAGACGCCTGA |
| | | AGGAGCTGGTGTACATGCGGTGTCTGGGCAACTCCTGGTCCAGCAAT |
| | | TGCCAGTGTACCTCTAACTCCCATGACAAGAGCAGAAAGCAGGTGAC |
| | | AGCCCAGCTGGAGCACCAGAAGGAGCAGCAGACCACAACCGATATGC |
| | | AGAAGCCCACCCAGTCTATGCACCAGGAGAATCTGACAGGCCATTGC |
| | | AGAGAGCCACCCCCTTGGAAGCACGAGGATAGCAAGCGCATCTATCA |
| | | TTTCGTGGAGGGCCAGTCTGTGCACTACGAGTGTATCCCCGGCTATA |
| | | AGGCCCTGCAGAGAGGCCCTGCTATCTCCATCTGCAAGATGAAGTGT |
| | | GGCAAGACCGGCTGGACACAGCCTCAGCTGACCTGCGTGGACGAGAG |
| | | GGAGCACCATCGGTTCCTGGCTAGCGAGGAGTCTCAGGGCTCCCGCA |
| | | ACTCTTCCCCTGAGAGCGAGACATCTTGTCCAATCACAACCACAGAT |
| | | TTTCCACAGCCCACCGAGACAACCGCTATGACAGAGACCTTCGTGCT |
| | | GACTATGGAATACAAATAATGA |
| 139 | hIL2- | ATGGGTTGGTCCTGCATCATCCTGTTCCTGGTCGCCACCGCCACTGG |
| | 2x(SG4)- | GGTCCACTCCgcacctacttcaagttctacaaagaaaacacagctac |
| | MMPcs1- | aactggagcatttacttctggatttacagatgattttgaatggaatt |
| | 2x(G4S)- | aataattacaagaatcccaaactcaccaggatgctcacatttaagtt |
| | hIL2Rbeta- | ttacatgcccaagaaggccacagaactgaaacatcttcagtgtctag |
| | hIgG1Fc | aagaagaactcaaacctctggaggaagtgctaaatttagctcaaagc |
| | (Construct U) | aaaaactttcacttaagacccagggacttaatcagcaatatcaacgt |
| | DNA | aatagttctggaactaaagggatctgaaacaacattcatgtgtgaat |
| | Sequence | atgctgatgagacagcaaccattgtagaatttctgaacagatggatt |
| | | accttttgtcaaagcatcatctcaacactgactTCTGGTGGCGGTGG |
| | | CTCTGGTGGCGGTGGCGGTCCTCTGGGTGTCAGAGGTGGTGGCGGTG |
| | | GCTCTGGTGGCGGTGGCTCTGCGGTGAATGGCACTTCCCAGTTCACA |
| | | TGCTTCTACAACTCGAGAGCCAACATCTCCTGTGTCTGGAGCCAAGA |
| | | TGGGGCTCTGCAGGACACTTCCTGCCAAGTCCATGCCTGGCCGGACA |
| | | GACGGCGGTGGAACCAAACCTGTGAGCTGCTCCCCGTGAGTCAAGCA |
| | | TCCTGGGCCTGCAACCTGATCCTCGGAGCCCCAGATTCTCAGAAACT |
| | | GACCACAGTTGACATCGTCACCCTGAGGGTGCTGTGCCGTGAGGGGG |
| | | TGCGATGGAGGGTGATGGCCATCCAGGACTTCAAGCCCTTTGAGAAC |
| | | CTTCGCCTGATGGCCCCCATCTCCCTCCAAGTTGTCCACGTGGAGAC |
| | | CCACAGATGCAACATAAGCTGGGAAATCTCCCAAGCCTCCCACTACT |
| | | TTGAAAGACACCTGGAGTTCGAGGCCCGGACGCTGTCCCCAGGCCAC |
| | | ACCTGGGAGGAGGCCCCCCTGCTGACTCTCAAGCAGAAGCAGGAATG |
| | | GATCTGCCTGGAGACGCTCACCCCAGACACCCAGTATGAGTTTCAGG |
| | | TGCGGGTCAAGCCTCTGCAAGGCGAGTTCACGACCTGGAGCCCCTGG |
| | | AGCCAGCCCCTGGCCTTCAGGACAAAGCCTGCAGCCCTTGGGAAGGA |
| | | CACCGACAAGACCCACACCTGTCCTCCATGTCCTGCTCCAGAATTGC |
| | | TCGGCGGACCCTCCGTGTTCCTGTTTCCTCCAAAGCCTAAGGACACC |
| | | CTGATGATCTCTCGGACCCCTGAAGTGACCTGCGTGGTGGTCGATGT |
| | | GTCTCACGAGGATCCCGAAGTGAAGTTCAATTGGTACGTGGACGGCG |
| | | TGGAAGTGCACAACGCCAAGACCAAGCCTAGAGAGGAACAGTACAAC |
| | | TCCACCTACAGAGTGGTGTCCGTGCTGACCGTGCTGCACCAGGATTG |
| | | GCTGAATGGCAAAGAGTACAAGTGCAAGGTGTCCAACAAGGCCCTGC |
| | | CTGCTCCTATCGAAAAGACCATCTCCAAGGCCAAGGGCCAGCCTAGG |
| | | GAACCCCAGGTTTACACCTTGCCTCCATCTCGGGACGAGCTGACCAA |
| | | GAACCAGGTGTCCCTGACCTGTCTGGTCAAGGGCTTCTACCCCTCCG |
| | | ATATCGCCGTGGAATGGGAGTCTAATGGCCAGCCTGAAAACAATTAC |
| | | AAGACAACCCCTCCTGTGCTGGACTCCGACGGCTCATTCTTCCTGTA |
| | | CAGCAAGCTGACAGTGGACAAGTCCAGATGGCAGCAGGGCAACGTGT |
| | | TCTCCTGCTCCGTGATGCATGAGGCCCTGCACAACCACTACACCCAG |
| | | AAGTCCCTGTCTCTGTCCCCTGGCTAATGA |
| 140 | hIL2- | ATGGGTTGGTCCTGCATCATCCTGTTCCTGGTCGCCACCGCCACTGG |
| | 2x(SG4)- | GGTCCACTCCgcacctacttcaagttctacaaagaaaacacagctac |
| | MMPcs1- | aactggagcatttacttctggatttacagatgattttgaatggaatt |
| | 2x(G4S)- | aataattacaagaatcccaaactcaccaggatgctcacatttaagtt |
| | hIL2Rgamma- | ttacatgcccaagaaggccacagaactgaaacatcttcagtgtctag |
| | hIgG1Fc | aagaagaactcaaacctctggaggaagtgctaaatttagctcaaagc |
| | DNA | aaaaactttcacttaagacccagggacttaatcagcaatatcaacgt |
| | sequence | aatagttctggaactaaagggatctgaaacaacattcatgtgtgaat |
| | | atgctgatgagacagcaaccattgtagaatttctgaacagatggatt |
| | | accttttgtcaaagcatcatctcaacactgactTCTGGTGGCGGTGG |
| | | CTCTGGTGGCGGTGGCGGTCCTCTGGGTGTCAGAGGTGGTGGCGGTG |
| | | GCTCTGGTGGCGGTGGCTCTCTGAACACGACAATTCTGACGCCCAAT |
| | | GGGAATGAAGACACCACAGCTGATTTCTTCCTGACCACTATGCCCAC |
| | | TGACTCCCTCAGTGTTTCCACTCTGCCCCTCCCAGAGGTTCAGTGTT |
| | | TTGTGTTCAATGTCGAGTACATGAATTGCACTTGGAACAGCAGCTCT |
| | | GAGCCCCAGCCTACCAACCTCACTCTGCATTATTGGTACAAGAACTC |
| | | GGATAATGATAAAGTCCAGAAGTGCAGCCACTATCTATTCTCTGAAG |
| | | AAATCACTTCTGGCTGTCAGTTGCAAAAAAAGGAGATCCACCTCTAC |
| | | CAAACATTTGTTGTTCAGCTCCAGGACCCACGGGAACCCAGGAGACA |
| | | GGCCACACAGATGCTAAAACTGCAGAATCTGGTGATCCCCTGGGCTC |
| | | CAGAGAACCTAACACTTCACAAACTGAGTGAATCCCAGCTAGAACTG |
| | | AACTGGAACAACAGATTCTTGAACCACTGTTTGGAGCACTTGGTGCA |
| | | GTACCGGACTGACTGGGACCACAGCTGGACTGAACAATCAGTGGATT |
| | | ATAGACATAAGTTCTCCTTGCCTAGTGTGGATGGGCAGAAACGCTAC |
| | | ACGTTTCGTGTTCGGAGCCGCTTTAACCCACTCTGTGGAAGTGCTCA |
| | | GCATTGGAGTGAATGGAGCCACCCAATCCACTGGGGGAGCAATACTT |
| | | CAAAAGAGAATCCTTTCCTGTTTGCATTGGAAGCCGACAAGACCCAC |
| | | ACCTGTCCTCCATGTCCTGCTCCAGAATTGCTCGGCGGACCCTCCGT |
| | | GTTCCTGTTTCCTCCAAAGCCTAAGGACACCCTGATGATCTCTCGGA |
| | | CCCCTGAAGTGACCTGCGTGGTGGTCGATGTGTCTCACGAGGATCCC |
| | | GAAGTGAAGTTCAATTGGTACGTGGACGGCGTGGAAGTGCACAACGC |
| | | CAAGACCAAGCCTAGAGAGGAACAGTACAACTCCACCTACAGAGTGG |
| | | TGTCCGTGCTGACCGTGCTGCACCAGGATTGGCTGAATGGCAAAGAG |
| | | TACAAGTGCAAGGTGTCCAACAAGGCCCTGCCTGCTCCTATCGAAAA |
| | | GACCATCTCCAAGGCCAAGGGCCAGCCTAGGGAACCCCAGGTTTACA |
| | | CCTTGCCTCCATCTCGGGACGAGCTGACCAAGAACCAGGTGTCCCTG |
| | | ACCTGTCTGGTCAAGGGCTTCTACCCCTCCGATATCGCCGTGGAATG |
| | | GGAGTCTAATGGCCAGCCTGAAAACAATTACAAGACAACCCCTCCTG |
| | | TGCTGGACTCCGACGGCTCATTCTTCCTGTACAGCAAGCTGACAGTG |
| | | GACAAGTCCAGATGGCAGCAGGGCAACGTGTTCTCCTGCTCCGTGAT |
| | | GCATGAGGCCCTGCACAACCACTACACCCAGAAGTCCCTGTCTCTGT |
| | | CCCCTGGCTAATGA |
| 141- | Not Used | |
| 149 | | |

Other DNA sequences

| 150 | Murine Ig | ATGGGTTGGTCCTGCATCATCCTGTTCCTGGTCGCCACCGCCACTGG |
|---|---|---|
| | kappa chain | GGTCCACTCC |
| | leader DNA | |
| | sequence | |
| 151 | Murine IL-2 | GCACCTACATCATCATCAACTTCATCCTCCACCGCTGAGGCTCAGCA |
| | DNA | ACAACAGCAACAACAGCAGCAGCAGCAGCAGCATCTGGAGCAGCTGC |
| | sequence | TGATGGACCTGCAGGAGCTGCTGTCCAGAATGGAGAACTACCGCAAT |
| | | CTGAAGCTGCCAAGGATGCTGACCTTCAAGTTTTATCTGCCCAAGCA |
| | | GGCCACAGAGCTGAAGGACCTGCAGTGCCTGGAGGATGAGCTGGGCC |
| | | CACTGAGGCACGTGCTGGACCTGACCCAGAGCAAGTCTTTCCAGCTG |
| | | GAGGATGCTGAGAACTTTATCTCCAATATCCGGGTGACCGTGGTGAA |
| | | GCTGAAGGGCAGCGACAACACATTCGAGTGCCAGTTTGACGATGAGT |
| | | CTGCCACCGTGGTGGATTTCCTGAGGCGGTGGATCGCTTTTTGTCAG |
| | | AGCATCATCTCCACAAGCCCTCAG |
| 152 | MMP | GGCCCACTGGGCGTGAGGGGT |
| | cleavage site | |
| | GPLGVRG | |
| | DNA | |
| | sequence | |
| 153 | Gly-Ser rich | TCTGGAGGAGGTGGCAGCGGAGGAGGAGGT |
| | linker DNA | |
| | sequence | |
| 154 | Murine IL- | GAGCTGTGCCTGTACGACCCCCCTGAGGTGCCCAATGCCACCTTCAA |
| | 2Ralpha DNA | GGCTCTGTCTTATAAGAACGGCACAATCCTGAATTGCGAGTGTAAGA |
| | sequence | GGGGCTTTAGACGCCTGAAGGAGCTGGTGTACATGCGGTGTCTGGGC |
| | | AACTCCTGGTCCAGCAATTGCCAGTGTACCTCTAACTCCCATGACAA |
| | | GAGCAGAAAGCAGGTGACAGCCCAGCTGGAGCACCAGAAGGAGCAGC |
| | | AGACCACAACCGATATGCAGAAGCCCACCCAGTCTATGCACCAGGAG |
| | | AATCTGACAGGCCATTGCAGAGAGCCACCCCCTTGGAAGCACGAGGA |
| | | TAGCAAGCGCATCTATCATTTCGTGGAGGGCCAGTCTGTGCACTACG |
| | | AGTGTATCCCCGGCTATAAGGCCCTGCAGAGAGGCCCTGCTATCTCC |
| | | ATCTGCAAGATGAAGTGTGGCAAGACCGGCTGGACACAGCCTCAGCT |
| | | GACCTGCGTGGACGAGAGGGAGCACCATCGGTTCCTGGCTAGCGAGG |
| | | AGTCTCAGGGCTCCCGCAACTCTTCCCCTGAGAGCGAGACATCTTGT |
| | | CCAATCACAACCACAGATTTTCCACAGCCCACCGAGACAACCGCTAT |
| | | GACAGAGACCTTCGTGCTGACTATGGAATACAAA |
| 155 | His tag DNA | CACCACCACCACCACCAC |
| | Sequence | |
| 156 | Stop codons | TAATGA |
| 157 | Murine IL-2 | GCACCTACATCATCATCAACTTCATCCTCCACCGCTGAGGCTCAGCA |
| | C140S DNA | ACAACAGCAACAACAGCAGCAGCAGCAGCAGCATCTGGAGCAGCTGC |
| | sequence | TGATGGACCTGCAGGAGCTGCTGTCCAGAATGGAGAACTACCGCAAT |
| | | CTGAAGCTGCCAAGGATGCTGACCTTCAAGTTTTATCTGCCCAAGCA |
| | | GGCCACAGAGCTGAAGGACCTGCAGTGCCTGGAGGATGAGCTGGGCC |
| | | CACTGAGGCACGTGCTGGACCTGACCCAGAGCAAGTCTTTCCAGCTG |
| | | GAGGATGCTGAGAACTTTATCTCCAATATCCGGGTGACCGTGGTGAA |
| | | GCTGAAGGGCAGCGACAACACATTCGAGTGCCAGTTTGACGATGAGT |
| | | CTGCCACCGTGGTGGATTTCCTGAGGCGGTGGATCGCTTTTTCCCAG |
| | | AGCATCATCTCCACAAGCCCTCA |
| 158 | Murine IgG1 | GGATGCAAACCCTGTATCTGTACCGTGCCCGAGGTCTCTTCCGTCTT |
| | T252M Fc | TATTTTCCCCCCCAAGCCTAAGGATGTGCTGATGATTACTCTGACCC |
| | domain DNA | CCAAGGTGACATGCGTGGTGGTGGACATCAGCAAGGACGATCCTGAG |
| | sequence | GTGCAGTTCTCTTGGTTTGTGGACGATGTGGAGGTGCACACCGCCCA |
| | | GACACAGCCAAGGGAGGAGCAGTTCAATAGCACCTTTCGGTCCGTGA |
| | | GCGAGCTGCCCATCATGCATCAGGATTGGCTGAATGGCAAGGAGTTC |
| | | AAGTGCAGAGTGAACTCTGCCGCTTTTCCCGCTCCTATCGAGAAGAC |
| | | CATCTCCAAGACAAAGGGCCGCCCAAAGGCTCCACAGGTGTACACCA |
| | | TCCCACCTCCAAAGGAGCAGATGGCTAAGGACAAGGTGTCTCTGACC |
| | | TGTATGATCACAGACTTCTTTCCTGAGGACATCACAGTGGAGTGGCA |
| | | GTGGAACGGCCAGCCTGCCGAGAACTATAAGAATACCCAGCCAATCA |
| | | TGGACACAGATGGCTCTTACTTCGTGTATTCCAAGCTGAACGTGCAG |
| | | AAGTCCAATTGGGAGGCTGGCAACACCTTTACATGTAGCGTGCTGCA |
| | | CGAAGGTCTGCATAACCATCATACCGAAAAATCACTGTCACACTCCC |
| | | CTGGA |
| 159 | Murine IgG1 | GGATGCAAACCCTGTATCTGTACCGTGCCCGAGGTCTCTTCCGTCTT |
| | Fc domain | TATTTTCCCCCCCAAGCCTAAGGATGTGCTGACTATTACTCTGACCC |
| | DNA | CCAAGGTGACATGCGTGGTGGTGGACATCAGCAAGGACGATCCTGAG |
| | sequence | GTGCAGTTCTCTTGGTTTGTGGACGATGTGGAGGTGCACACCGCCCA |
| | | GACACAGCCAAGGGAGGAGCAGTTCAATAGCACCTTTCGGTCCGTGA |
| | | GCGAGCTGCCCATCATGCATCAGGATTGGCTGAATGGCAAGGAGTTC |
| | | AAGTGCAGAGTGAACTCTGCCGCTTTTCCCGCTCCTATCGAGAAGAC |
| | | CATCTCCAAGACAAAGGGCCGCCCAAAGGCTCCACAGGTGTACACCA |
| | | TCCCACCTCCAAAGGAGCAGATGGCTAAGGACAAGGTGTCTCTGACC |
| | | TGTATGATCACAGACTTCTTTCCTGAGGACATCACAGTGGAGTGGCA |
| | | GTGGAACGGCCAGCCTGCCGAGAACTATAAGAATACCCAGCCAATCA |
| | | TGGACACAGATGGCTCTTACTTCGTGTATTCCAAGCTGAACGTGCAG |
| | | AAGTCCAATTGGGAGGCTGGCAACACCTTTACATGTAGCGTGCTGCA |
| | | CGAAGGTCTGCATAACCATCATACCGAAAAATCACTGTCACACTCCC |
| | | CTGGAAAA |
| 160 | Human IL-2 | GCTCCTACATCCTCCAGCACCAAGAAAACCCAGCTGCAGTTGGAGCA |
| | C125S DNA | TCTGCTGCTGGACCTGCAGATGATCCTGAACGGCATCAACAACTACA |
| | sequence | AGAACCCCAAGCTGACCCGGATGCTGACCTTCAAGTTCTACATGCCC |
| | | AAGAAGGCCACCGAGCTGAAACATCTGCAGTGCCTGGAAGAGGAACT |
| | | GAAGCCCCTGGAAGAAGTGCTGAATCTGGCCCAGTCCAAGAACTTCC |
| | | ACCTGAGGCCTCGGGACCTGATCTCCAACATCAACGTGATCGTGCTC |
| | | GAGCTGAAGGGCTCCGAGACAACCTTCATGTGCGAGTACGCCGACGA |
| | | GACAGCTACCATCGTGGAATTTCTGAACCGGTGGATCACCTTCAGCC |
| | | AGTCCATCATCAGCACCCTGACA |
| 161 | Human IgG1 | GACAAGACCCACACCTGTCCTCCATGTCCTGCTCCAGAATTGCTCGG |
| | Fc domain | CGGACCCTCCGTGTTCCTGTTTCCTCCAAAGCCTAAGGACACCCTGA |
| | DNA | TGATCTCTCGGACCCCTGAAGTGACCTGCGTGGTGGTCGATGTGTCT |
| | sequence | CACGAGGATCCCGAAGTGAAGTTCAATTGGTACGTGGACGGCGTGGA |
| | | AGTGCACAACGCCAAGACCAAGCCTAGAGAGGAACAGTACAACTCCA |
| | | CCTACAGAGTGGTGTCCGTGCTGACCGTGCTGCACCAGGATTGGCTG |
| | | AATGGCAAAGAGTACAAGTGCAAGGTGTCCAACAAGGCCCTGCCTGC |
| | | TCCTATCGAAAAGACCATCTCCAAGGCCAAGGGCCAGCCTAGGGAAC |
| | | CCCAGGTTTACACCTTGCCTCCATCTCGGGACGAGCTGACCAAGAAC |
| | | CAGGTGTCCCTGACCTGTCTGGTCAAGGGCTTCTACCCCTCCGATAT |
| | | CGCCGTGGAATGGGAGTCTAATGGCCAGCCTGAAAACAATTACAAGA |
| | | CAACCCCTCCTGTGCTGGACTCCGACGGCTCATTCTTCCTGTACAGC |
| | | AAGCTGACAGTGGACAAGTCCAGATGGCAGCAGGGCAACGTGTTCTC |
| | | CTGCTCCGTGATGCATGAGGCCCTGCACAACCACTACACCCAGAAGT |
| | | CCCTGTCTCTGTCCCCTGGC |
| 162 | Human IL-2 | GCACCTACTTCAAGTTCTACAAAGAAAACACAGCTACAACTGGAGCA |
| | DNA | TTTACTTCTGGATTTACAGATGATTTTGAATGGAATTAATAATTACA |
| | sequence | AGAATCCCAAACTCACCAGGATGCTCACATTTAAGTTTTACATGCCC |
| | | AAGAAGGCCACAGAACTGAAACATCTTCAGTGTCTAGAAGAAGAACT |
| | | CAAACCTCTGGAGGAAGTGCTAAATTTAGCTCAAAGCAAAAACTTTC |
| | | ACTTAAGACCCAGGGACTTAATCAGCAATATCAACGTAATAGTTCTG |
| | | GAACTAAAGGGATCTGAAACAACATTCATGTGTGAATATGCTGATGA |
| | | GACAGCAACCATTGTAGAATTTCTGAACAGATGGATTACCTTTTGTC |
| | | AAAGCATCATCTCAACACTGACT |
| 163 | Human IgG1 | GACAAAACTCACACATGCCCACCGTGCCCAGCACCTGAACTCCTGGG |
| | K392D | GGGACCGTCAGTCTTCCTCTTCCCCCCAAAACCCAAGGACACCCTCA |
| | K409D Fc | TGATCTCCCGGACCCCTGAGGTCACATGCGTGGTGGTGGACGTGAGC |
| | domain DNA | CACGAAGACCCTGAGGTCAAGTTCAACTGGTACGTGGACGGCGTGGA |
| | sequence | GGTGCATAATGCCAAGACAAAGCCGCGGGAGGAGCAGTACAACAGCA |
| | | CGTACCGTGTGGTCAGCGTCCTCACCGTCCTGCACCAGGACTGGCTG |
| | | AATGGCAAGGAGTACAAGTGCAAGGTCTCCAACAAAGCCCTCCCAGC |
| | | CCCCATCGAGAAAACCATCTCCAAAGCCAAAGGGCAGCCCCGAGAAC |
| | | CACAGGTGTACACCCTGCCCCCATCCCGGGATGAGCTGACCAAGAAC |
| | | CAGGTCAGCCTGACCTGCCTGGTCAAAGGCTTCTATCCCAGCGACAT |
| | | CGCCGTGGAGTGGGAGAGCAATGGGCAGCCGGAGAACAACTACGACA |
| | | CCACGCCTCCCGTGCTGGACTCCGACGGCTCCTTCTTCCTCTATAGC |
| | | GACCTCACCGTGGACAAGAGCAGGTGGCAGCAGGGGAACGTCTTCTC |
| | | ATGCTCCGTGATGCATGAGGCTCTGCACAACCACTACACGCAGAAGA |
| | | GCCTCTCCCTGTCTCCGGGT |
| 164 | Human IL- | GAGCTCTGTGACGATGACCCGCCAGAGATCCCACACGCCACATTCAA |
| | 2RalphaDNA | AGCCATGGCCTACAAGGAAGGAACCATGTTGAACTGTGAATGCAAGA |
| | Sequence | GAGGTTTCCGCAGAATAAAAAGCGGGTCACTCTATATGCTCTGTACA |
| | | GGAAACTCTAGCCACTCGTCCTGGGACAACCAATGTCAATGCACAAG |
| | | CTCTGCCACTCGGAACACAACGAAACAAGTGACACCTCAACCTGAAG |
| | | AACAGAAAGAAAGGAAAACCACAGAAATGCAAAGTCCAATGCAGCCA |
| | | GTGGACCAAGCGAGCCTTCCAGGTCACTGCAGGGAACCTCCACCATG |
| | | GGAAAATGAAGCCACAGAGAGAATTTATCATTTCGTGGTGGGGCAGA |
| | | TGGTTTATTATCAGTGCGTCCAGGGATACAGGGCTCTACACAGAGGT |
| | | CCTGCTGAGAGCGTCTGCAAAATGACCCACGGGAAGACAAGGTGGAC |
| | | CCAGCCCCAGCTCATATGCACAGGTGAAATGGAGACCAGTCAGTTTC |
| | | CAGGTGAAGAGAAGCCTCAGGCAAGCCCCGAAGGCCGTCCTGAGAGT |
| | | GAGACTTCCTGCCTCGTCACAACAACAGATTTTCAAATACAGACAGA |
| | | AATGGCTGCAACCATGGAGACGTCCATATTTACAACAGAGTACCAG |
| 165 | Gly-Ser | TCTGGTGGCGGTGGCTCTGGTGG |
| | Linker DNA | CGGTGGC |
| | sequence | |
| 166 | Human MMP | GGTCCTCTGGGTGTCAGAGGT |
| | Cleavage Site | |
| | DNA | |
| | sequence | |
| 167- | Not Used | |
| 200 | | |
| Additional Protease-cleavable sequences | | |
| SEQ | | |
| ID NO | Cleavable by | Sequence |
| 201 | MMP7 | KRALGLPG |
| 202 | MMP7 | (DE)₈RPLALWRS(DR)₈ |
| 203 | MMP9 | PR(S/T)(L/I)(S/T) |
| 204 | MMP9 | LEATA |
| 205 | MMP11 | GGAANLVRGG |
| 206 | MMP14 | SGRIGFLRTA |
| 207 | MMP | PLGLAG |
| 208 | MMP | PLGLAX |
| 209 | MMP | PLGC(me)AG |
| 210 | MMP | ESPAYYTA |
| 211 | MMP | RLQLKL |
| 212 | MMP | RLQLKAC |
| 213 | MMP, | EP(Cit)G(Hof)YL |
| | MMP9, | |
| | MMP14 | |
| 214 | Urokinase | SGRSA |
| | plasminogen | |
| | activator | |
| | (uPA) | |
| 215 | Urokinase | DAFK |
| | plasminogen | |
| | activator | |
| | (uPA) | |
| 216 | Urokinase | GGG RR |
| | plasminogen | |
| | activator | |
| | (uPA) | |
| 217 | Lysomal | GFLG |
| | Enzyme | |
| 218 | Lysomal | ALAL |
| | Enzyme | |
| 219 | Lysomal | FK |
| | Enzyme | |
| 220 | Cathepsin B | NLL |
| 221 | Cathepsin D | PIC(Et)FF |
| 222 | Cathepsin K | GGPRGLPG |
| 223 | Prostate | HSSKLQ |
| | Specific | |
| | Antigen | |
| 224 | Prostate | HSSKLQL |
| | Specific | |
| | Antigen | |
| 225 | Prostate | HSSKLQEDA |
| | Specific | |
| | Antigen | |
| 226 | Herpes | LVLASSSFGY |
| | Simplex Virus | |
| | Protease | |
| 227 | HIV Protease | GVSQNYPIVG |
| 228 | CMV | GVVQASCRLA |
| | Protease | |
| 229 | Thrombin | F(Pip)RS |
| 230 | Thrombin | DPRSFL |
| 231 | Thrombin | PPRSFL |
| 232 | Caspase-3 | DEVD |
| 233 | Caspase-3 | DEVDP |
| 234 | Caspase-3 | KGSGDVEG |
| 235 | Interleukin 10 | GWEHDG |
| | converting | |
| | enzyme | |
| 236 | Enterokinase | EDDDDKA |
| 237 | FAP | KQEQNPGST |
| 238 | Kallikrein 2 | GKAFRR |
| 239 | Plasmin | DAFK |
| 240 | Plasmin | DVLK |
| 241 | Plasmin | DAFK |
| 242 | TOP | ALLLALL |

### Definitions

As used herein, a “cytokine polypeptide sequence” refers to a polypeptide sequence (which may be part of a larger sequence, e.g., a fusion polypeptide) with significant sequence identity to a wild-type cytokine and which can bind and activate a cytokine receptor when separated from an inhibitory polypeptide sequence. In some embodiments, a cytokine polypeptide sequence has at least 80, 85, 90, 95, 97, 98, or 99 percent identity to the sequence of a wild-type cytokine, e.g., a wild-type human cytokine. In some embodiments, a cytokine polypeptide sequence has no more than one, two, three, four, five, six, seven, eight, nine, or ten amino acid differences from a wild-type cytokine, e.g., a wild-type human cytokine. Cytokines include but are not limited to chemokines. Exemplary cytokine polypeptide sequences are provided in Table 1. This definition applies to IL-2 polypeptide sequences with substitution of “IL-2” for “cytokine.”

As used herein, an “inhibitory polypeptide sequence” is a sequence in a cytokine prodrug that inhibits the activity of the cytokine polypeptide sequence in the prodrug. The inhibitory polypeptide sequence binds the cytokine polypeptide sequence, and such binding is reduced or eliminated by action of an appropriate protease on the protease-cleavable polypeptide sequence. Exemplary inhibitory polypeptide sequences are provided in Table 1.

As used herein, a “protease-cleavable polypeptide sequence” is a sequence that is a substrate for cleavage by a protease. The protease-cleavable polypeptide sequence is located in a cytokine prodrug such that its cleavage reduces or eliminates binding of the inhibitory polypeptide sequence to the cytokine polypeptide sequence.

As used herein, a protease-cleavable polypeptide sequence “is recognized by” a given protease or class thereof if exposing a polypeptide comprising the protease-cleavable polypeptide sequence to the protease under conditions permissive for cleavage by the protease results in a significantly greater amount of cleavage than is seen for a control polypeptide having an unrelated sequence, and/or if the protease-cleavable polypeptide sequence corresponds to a known recognition sequence for the protease (e.g., as described elsewhere herein for various exemplary proteases).

As used herein, a “pharmacokinetic modulator” is a moiety that extends the in vivo half-life of a cytokine prodrug. The pharmacokinetic modulator may be a fused domain in a cytokine prodrug or may be a chemical entity attached post-translationally. The attachment may be, but is not necessarily, covalent. Exemplary pharmacokinetic modulator polypeptide sequences are provided in Table 1. Exemplary non-polypeptide pharmacokinetic modulators are described elsewhere herein.

As used herein, an “immunoglobulin constant domain” refers to a domain that occurs in or has significant sequence identity to a domain of a constant region of an immunoglobulin, such as an IgG. Exemplary constant domains are C_{H}2 and C_{H}3 domains. Unless indicated otherwise, a polypeptide or prodrug comprising an immunoglobulin constant domain may comprise more than one immunoglobulin constant domain. In some embodiments, an immunoglobulin constant domain has at least 80, 85, 90, 95, 97, 98, or 99 percent identity to the sequence of a wild-type immunoglobulin constant domain, e.g., a wild-type human immunoglobulin constant domain. In some embodiments, an immunoglobulin constant domain has no more than one, two, three, four, five, six, seven, eight, nine, or ten amino acid differences from a wild-type immunoglobulin constant domain, e.g., a wild-type human immunoglobulin constant domain. In some embodiments, immunoglobulin constant domain has an identical sequence to a wild-type immunoglobulin constant domain, e.g., a wild-type human immunoglobulin constant domain. Exemplary immunoglobulin constant domains are contained within sequences provided in Table 1. This definition applies to C_{H}2 and C_{H}3 domains, respectively, with substitution of “C_{H}2” or “C_{H}3” for “immunoglobulin constant,” with the qualification that a C_{H}2 domain sequence does not have greater percent identity to a non-C_{H}2 immunoglobulin constant domain wild-type sequence than to a C_{H}2 domain wild-type sequence, and a C_{H}3 domain sequence does not have greater percent identity to a non-C_{H}3 immunoglobulin constant domain wild-type sequence than to a C_{H}3 domain wild-type sequence. These definitions also include domains having minor truncations relative to wild-type sequences, to the extent that the truncation does not abrogate substantially normal folding of the domain.

As used herein, a “immunoglobulin Fc region” refers to a region of an immunoglobulin heavy chain comprising a C_{H}2 and a C_{H}3 domain, as defined above. The Fc region does not include a variable domain or a C_{H}1 domain.

As used herein, a given component is “between” a first component and a second component if the first component is on one side of the given component and the second component is on the other component, e.g., in the primary sequence of a polypeptide. This term does not require immediate adjacency. Thus, in the structure 1-2-3-4, 2 is between 1 and 4, and is also between 1 and 3.

As used herein, a “domain” may refer, depending on the context, to a structural domain of a polypeptide or to a functional assembly of at least one domain (but possibly a plurality of structural domains). For example, a C_{H}2 domain refers to a part of a sequence that qualifies as such. An immunoglobulin cytokine-binding domain may comprise VH and VL structural domains.

As used herein, “denatured collagen” encompasses gelatin and cleavage products resulting from action of an MMP on collagen, and more generally refers to a form of collagen or fragments thereof that does not exist in the native structure of full-length collagen.

As used herein, a “cytokine-binding domain of a cytokine receptor” refers to an extracellular portion of a cytokine receptor, or a fragment or truncation thereof that can bind a cytokine polypeptide sequence. In some embodiments, the sequence of a cytokine binding domain of a cytokine receptor has at least 80, 85, 90, 95, 97, 98, or 99 percent identity to the sequence of a cytokine binding domain of wild-type cytokine receptor, e.g., a cytokine binding domain of a wild-type human cytokine receptor. Exemplary sequences of a cytokine binding domain of a cytokine receptor are provided in Table 1. This definition applies to IL-2-binding domains of an IL-2 receptor with substitution of “IL-2” for “cytokine.”

As used herein, a “cytokine-binding immunoglobulin domain” refers to one or more immunoglobulin variable domains (e.g., a VH and a VL domain) that can bind a cytokine polypeptide sequence. Exemplary sequences of a cytokine-binding immunoglobulin domain are provided in Table 1. This definition applies to IL-2-binding immunoglobulin domains with substitution of “IL-2” for “cytokine.”

As used herein, “substantially” and other grammatical forms thereof mean sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “plurality” can be 2, 3, 4, 5, 6, 7, 8, 9, 10, or more.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence QLYV comprises a sequence with 100% identity to the sequence QLY because an alignment would give 100% identity in that there are matches to all three positions of the second sequence. Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

As used herein, a “subject” refers to any member of the animal kingdom. In some embodiments, “subject” refers to humans. In some embodiments, “subject” refers to non-human animals. In some embodiments, “subject” refers to primates. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically-engineered animal, and/or a clone. In certain embodiments of the present invention the subject is an adult, an adolescent or an infant. In some embodiments, the terms “individual” or “patient” are used and are intended to be interchangeable with “subject”.

### Cytokine Polypeptide Sequence

The cytokine polypeptide sequence may be a wild-type cytokine polypeptide sequence or a sequence with one or more differences from the wild-type cytokine polypeptide sequence. In some embodiments, the cytokine polypeptide sequence is a human cytokine polypeptide sequence (which may be wild-type or may have one or more differences). In some embodiments, the cytokine comprises a modification to prevent disulfide bond formation, and optionally otherwise comprises wild-type sequence. In some embodiments, the cytokine polypeptide sequence has at least 80, 85, 90, 95, 97, 98, or 99 percent identity to the sequence of a wild-type cytokine polypeptide sequence or to a cytokine polypeptide sequence in Table 1. In some embodiments, the cytokine is a dimeric cytokine, e.g., a heterodimeric cytokine. The monomers may be linked as a fusion protein, e.g., with a linker, or by a covalent bond (e.g., disulfide bond), or by a noncovalent interaction.

IL-2

In some embodiments, the cytokine polypeptide sequence is an IL-2 polypeptide sequence. The IL-2 polypeptide sequence may be a wild-type IL-2 polypeptide sequence or a sequence with one or more differences from the wild-type IL-2 polypeptide sequence. In some embodiments, the IL-2 polypeptide sequence is a human IL-2 polypeptide sequence (which may be wild-type or may have one or more differences). In some embodiments, the IL-2 comprises a modification to prevent disulfide bond formation (e.g., the sequence of aldesleukin (marketed as Proleukin®), and optionally otherwise comprises wild-type sequence. In some embodiments, the IL-2 polypeptide sequence has at least 80, 85, 90, 95, 97, 98, or 99 percent identity to the sequence of a wild-type IL-2 polypeptide sequence or to a IL-2 polypeptide sequence in Table 1.

### Inhibitory Polypeptide Sequence

Various types of inhibitory polypeptide sequences may be used in a cytokine prodrug according to the disclosure. In some embodiments, the inhibitory polypeptide sequence comprises a cytokine-binding domain.

The cytokine-binding domain may be the cytokine-binding domain of a cytokine receptor. The cytokine-binding domain of a cytokine receptor may be provided as an extracellular portion of the cytokine receptor or a portion thereof sufficient to bind the cytokine polypeptide sequence of the cytokine prodrug. In some embodiments, the cytokine-binding domain of a cytokine receptor has at least 80, 85, 90, 95, 97, 98, or 99 percent identity to the sequence of a wild-type cytokine-binding domain of a cytokine receptor, e.g., a wild-type cytokine-binding domain of a human cytokine receptor.

The cytokine-binding domain may be a fibronectin cytokine-binding domain. In some embodiments, the fibronectin cytokine-binding domain has at least 80, 85, 90, 95, 97, 98, or 99 percent identity to the sequence of a wild-type fibronectin cytokine-binding domain of a cytokine receptor, e.g., a wild-type human fibronectin cytokine-binding domain.

The cytokine-binding domain may be an immunoglobulin cytokine-binding domain. The immunoglobulin cytokine-binding domain may be an Fv, scFv, Fab, or other immunoglobulin sequence having antigen-binding activity for the cytokine polypeptide sequence.

Additional examples of inhibitory polypeptide sequences that may be provided to inhibit the cytokine polypeptide sequence of the cytokine prodrug are anticalins, affilins, affibody molecules, affimers, affitins, alphabodies, avimers, DARPins, fynomers, kunitz domain peptides, monobodies, and binding domains based on other engineered scaffolds such as SpA, GroEL, lipocallin and CTLA4 scaffolds.

IL-2 Inhibitory Polypeptide Sequence

In cytokine prodrugs comprising an IL-2 polypeptide sequence, the inhibitory polypeptide sequence may be an IL-2 inhibitory polypeptide sequence of any of the types described above. In some embodiments, the IL-2 inhibitory polypeptide sequence is an immunoglobulin IL-2 inhibitory polypeptide sequence. In some embodiments, the IL-2 inhibitory polypeptide sequence comprises an anti-IL-2 antibody or a functional fragment thereof. In some embodiments, the immunoglobulin IL-2 inhibitory polypeptide sequence comprises a set of six anti-IL2 hypervariable regions (HVRs) set forth in Table 1 (e.g., SEQ ID NOs: 34-39 or 250-255). In some embodiments, the IL-2 inhibitory polypeptide sequence comprises a set of anti-IL2 VH and VL sequences having at least 80, 85, 90, 95, 97, 98, or 99 percent identity to the sequence of a set of anti-IL2 VH and VL sequences set forth in Table 1, either as individual sequences or as part of an scFv. In some embodiments, the IL-2 inhibitory polypeptide sequence comprises a set of anti-IL2 VH and VL sequences having the sequence of a set of anti-IL2 VH and VL sequences set forth in Table 1, either as individual sequences or as part of an scFv. Exemplary IL-2 inhibitory polypeptide sequences include SEQ ID NOS: 10-31, 40-51, and 247, and a combination of SEQ ID NOs 32 and 33 or a combination of SEQ ID NOs 248 and 249.

### Protease-Cleavable Sequence

The protease-cleavable sequence may be selected from sequences cleavable by various types of proteases, e.g., a metalloprotease, a serine protease, a cysteine protease, an aspartate protease, a threonine protease, a glutamate protease, a gelatinase, an asparagine peptide lyase, a cathepsin, a kallikrein, a plasmin, a collagenase, a hK1, a hK10, a hK15, a stromelysin, a Factor Xa, a chymotrypsin-like protease, a trypsin-like protease, a elastase-like protease, a subtilisin-like protease, an actinidain, a bromelain, a calpain, a caspase, a Mir 1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, an ADAM 10, an ADAM17, an ADAM 12, an urokinase plasminogen activator (uPA), an enterokinase, a prostate-specific target (PSA, hK3), an interleukin-1b converting enzyme, a thrombin, a FAP (FAP-a), a dipeptidyl peptidase, or dipeptidyl peptidase IV (DPPIV/CD26), a type II transmembrane serine protease (TTSP), a neutrophil elastase, a proteinase 3, a mast cell chymase, a mast cell tryptase, or a dipeptidyl peptidase. In some embodiments, the protease-cleavable sequence comprises the sequence of any one of those in Table 1 (e.g., SEQ ID NOs: 80-90 or 201-242), or a variant having one or two mismatches relative to the sequence of any one of those in Table 1 (e.g., SEQ ID NOs: 80-90 or 201-242). Proteases generally do not require an exact copy of the recognition sequence, and as such, the exemplary sequences may be varied at a portion of their amino acid positions. In some embodiments, the protease-cleavable sequence comprises a sequence that matches an MMP consensus sequence, such as any one of SEQ ID NOs: 91-94.

See Choi et al., Theranostics 2012; 2(2):156-178, for further discussion of proteases and their recognition sites in the context of prodrugs.

Matrix Metalloprotease-Cleavable Sequence

In some embodiments, the protease-cleavable sequence is a matrix metalloprotease (MMP)-cleavable sequence. Exemplary MMP-cleavable sequences are provided in Table 1. In some embodiments, the MMP-cleavable sequence is cleavable by a plurality of MMPs and/or one or more of MMP-1, MMP-2, MMP-3, MMP-7, MMP-8, MMP-9, MMP-12, MMP-13, and/or MMP-14. Table 1, e.g., SEQ ID NOs: 80-90, provides exemplary MMP-cleavable sequences.

### Pharmacokinetic Modulators

In some embodiments, the cytokine prodrug comprises a pharmacokinetic modulator. The pharmacokinetic modulator may be covalently or noncovalently associated with the cytokine prodrug. The pharmacokinetic modulator can extend the half-life of the cytokine prodrug and optionally the cytokine polypeptide sequence, e.g., so that fewer doses are necessary and less of the prodrug needs to be administered over time to achieve a desired result. In some embodiments, the pharmacokinetic modulator comprises a polypeptide (see examples below). In some embodiments, the pharmacokinetic modulator comprises a non-polypeptide moiety (e.g., polyethylene glycol, a polysaccharide, or hyaluronic acid). A non-polypeptide moiety can be associated with the prodrug using known approaches, e.g., conjugation to the prodrug; for example, a reactive amino acid residue can be used or added to the prodrug to facilitate conjugation.

In some embodiments, the pharmacokinetic modulator alters the size, shape, and/or charge of the prodrug, e.g., in a manner that reduces clearance. For example, a pharmacokinetic modulator with a negative charge may inhibit renal clearance. In some embodiments, the pharmacokinetic modulator increases the hydrodynamic volume of the prodrug. In some embodiments, the pharmacokinetic modulator reduces renal clearance, e.g., by increasing the hydrodynamic volume of the prodrug.

In some embodiments, the cytokine prodrug comprising the pharmacokinetic modulator (e.g., any of the pharmacokinetic modulators described herein) has a molecular weight of at least 70 kDa, e.g., at least 75 or 80 kDa.

For further discussion of various approaches for providing a pharmacokinetic modulator, see, e.g., Strohl, BioDrugs 29:215-19 (2015) and Podust et al., J. Controlled Release 240:52-66 (2016).

Polypeptide Pharmacokinetic Modulators

In some embodiments, the pharmacokinetic modulator comprises a polypeptide, e.g., an immunoglobulin sequence (see exemplary embodiments below), an albumin, a CTP (a negatively-charged carboxy-terminal peptide of the chorionic gonadotropin β-chain that undergoes sialylation in vivo and in appropriate host cells), an inert polypeptide (e.g., an unstructured polypeptide such as an XTEN, a polypeptide comprising the residues Ala, Glu, Gly, Pro, Ser, and Thr), a transferrin, a homo-amino-acid polypeptide, or an elastin-like polypeptide.

Exemplary polypeptide sequences suitable for use as a pharmacokinetic modulator are provided in Table 1 (e.g., any one of SEQ ID NOs: 70-74). In some embodiments, the pharmacokinetic modulator has at least 80, 85, 90, 95, 97, 98, or 99 percent identity to the sequence of a pharmacokinetic modulator in Table 1 (e.g., any one of SEQ ID NOs: 70-74).

In any embodiment where the pharmacokinetic modulator comprises a polypeptide sequence from an organism, the polypeptide sequence may be a human polypeptide sequence.

Immunoglobulin Pharmacokinetic Modulators

In some embodiments, the pharmacokinetic modulator comprises an immunoglobulin sequence, e.g., one or more immunoglobulin constant domains. In some embodiments, the pharmacokinetic modulator comprises an Fc region. The immunoglobulin sequence (e.g., one or more immunoglobulin constant domains or Fc region) may be a human immunoglobulin sequence. The immunoglobulin sequence (e.g., one or more immunoglobulin constant domains or Fc region) may have at least 80, 85, 90, 95, 97, 98, or 99 percent identity to the sequence of a wild-type immunoglobulin sequence (e.g., one or more immunoglobulin constant domains or Fc region), such as a wild-type human immunoglobulin sequence. In any of such embodiments, the immunoglobulin sequence may comprise an IgG sequence (e.g., IgG1, IgG2, IgG3, or IgG4). Exemplary immunoglobulin pharmacokinetic modulator sequences include SEQ ID NOS: 70-74 and the combination of SEQ ID NOs 256 and 257.

### Arrangement of Components

The recitation of components of a cytokine prodrug herein does not imply any particular order beyond what is explicitly stated (for example, it may be explicitly stated that a protease-cleavable sequence is between the cytokine polypeptide sequence and the inhibitory polypeptide sequence). The components of the cytokine prodrug may be arranged in various ways to provide properties suitable for a particular use. The components of the cytokine prodrug may be all in one polypeptide chain or they may be in a plurality of polypeptide chains bridged by covalent bonds, such as disulfide bonds. For example, where a pharmacokinetic modulator comprises an Fc, one or more components may be bound to one chain while one or more other components may be bound to the other chain. The Fc may be a heterodimeric Fc, such as a knob-into-hole Fc (in which one chain of the Fc comprises knob mutations and the other chain of the Fc comprises hole mutations). For an exemplary general discussion of knob and hole mutations, see, e.g., Xu et al., mAbs 7:1, 231-242 (2015). Exemplary knob mutations (e.g., for a human IgG1 Fc) are K360E/K409W. Exemplary hole mutations (e.g., for a human IgG1 Fc) are Q347R/D399V/F405T. See SEQ ID NOs: 256 and 257.

For example, a pharmacokinetic modulator can be present on the same side of the protease-cleavable sequence as the cytokine polypeptide sequence, meaning that cleavage of the protease-cleavable sequence does not separate the pharmacokinetic modulator from the cytokine polypeptide sequence. Examples of such structures include CY-PM-CL-IN, IN-CL-CY-PM, and any other permutation (or variation in which additional elements are included between, before, or after the listed components) in which CL is not between CY and PM, where CY is the cytokine polypeptide sequence, PM is the pharmacokinetic modulator, CL is the protease-cleavable sequence, and IN is the inhibitory polypeptide sequence. In such embodiments, the pharmacokentic modulator will modulate the pharmacokinetics of both the prodrug and the active cytokine polypeptide sequence. In some embodiments, the pharmacokinetic modulator is an Fc, in which case the components preceding and following PM in the exemplary structures above may be bound to the same or different chains of the Fc, as discussed above.

In some embodiments, a pharmacokinetic modulator is present on the same side of the protease-cleavable sequence as the inhibitory polypeptide sequence, meaning that cleavage of the protease-cleavable sequence does separate the pharmacokinetic modulator from the cytokine polypeptide sequence. Such embodiments can be useful to provide a longer half-life for the prodrug than for the active form.

### Exemplary Prodrugs

IL-2

The following table shows exemplary combinations of components according to certain embodiments of the disclosed cytokine prodrugs. The numbers indicate SEQ ID NOs for a given component. CY is the cytokine polypeptide sequence, CL is the protease-cleavable sequence, and IN is the inhibitory polypeptide sequence, and, where present, PM is the pharmacokinetic modulator. Where a range is given, any one of the listed SEQ ID NOs may be selected. Where two SEQ ID NOs are recited conjunctively (using “and”), both SEQ ID NOs are present and can function together (they may or may not be fused to each other, optionally with an intervening linker, or bridged, e.g., by a covalent bond). For example, SEQ ID NOs 32 and 33 are VL and VH domains that can function together to form a cytokine-binding immunoglobulin domain, as are SEQ ID NOs 248 and 249. SEQ ID NOs 256 and 257 are Fc polypeptide chains for forming a heterodimeric knob-into-hole Fc that can serve as a pharmacokinetic modulator. The components may be arranged in any manner consistent with the disclosure, e.g., as indicated elsewhere herein. In some embodiments, a cytokine prodrug comprises a combination of sequences as set forth in Table 2.

TABLE 2
Exemplary IL-2 prodrugs

| CY | CL | IN | PM |
|---|---|---|---|
| 1-2 | 80-90 or 201-242 | 10-16, 30, 31, | |
| | | 40-51, 247, | |
| | | (32 and 33), | |
| | | or (248 and | |
| | | 249) | |
| 1-2 | 80-90 or 201-242 | 10-16, 30, 31, | 70-74 or |
| | | 40-51, 247, | (256 and |
| | | (32 and 33), | 257) |
| | | or (248 and | |
| | | 249) | |
| 1 | 80-90 or 201-242 | 10 | |
| 1 | 80-90 or 201-242 | 11 | |
| 1 | 80-90 or 201-242 | 12 | |
| 1 | 80-90 or 201-242 | 13 | |
| 1 | 80-90 or 201-242 | 14 | |
| 1 | 80-90 or 201-242 | 15 | |
| 1 | 80-90 or 201-242 | 16 | |
| 1 | 80-90 or 201-242 | 20 | |
| 1 | 80-90 or 201-242 | 21 | |
| 1 | 80-90 or 201-242 | 22 | |
| 1 | 80-90 or 201-242 | 23 | |
| 1 | 80-90 or 201-242 | 24 | |
| 1 | 80-90 or 201-242 | 25 | |
| 1 | 80-90 or 201-242 | 26 | |
| 1 | 80-90 or 201-242 | 27 | |
| 1 | 80-90 or 201-242 | 28 | |
| 1 | 80-90 or 201-242 | 29 | |
| 1 | 80-90 or 201-242 | 30 | |
| 1 | 80-90 or 201-242 | 31 | |
| 1 | 80-90 or 201-242 | 32 and 33 | |
| 1 | 80-90 or 201-242 | 40 | |
| 1 | 80-90 or 201-242 | 41 | |
| 1 | 80-90 or 201-242 | 42 | |
| 1 | 80-90 or 201-242 | 43 | |
| 1 | 80-90 or 201-242 | 44 | |
| 1 | 80-90 or 201-242 | 45 | |
| 1 | 80-90 or 201-242 | 46 | |
| 1 | 80-90 or 201-242 | 47 | |
| 1 | 80-90 or 201-242 | 48 | |
| 1 | 80-90 or 201-242 | 49 | |
| 1 | 80-90 or 201-242 | 50 | |
| 1 | 80-90 or 201-242 | 51 | |
| 1 | 80-90 or 201-242 | 247 | |
| 1 | 80-90 or 201-242 | 248 and 249 | |
| 1 | 80-90 or 201-242 | 10 | 70 |
| 1 | 80-90 or 201-242 | 10 | 71 |
| 1 | 80-90 or 201-242 | 10 | 72 |
| 1 | 80-90 or 201-242 | 10 | 73 |
| 1 | 80-90 or 201-242 | 10 | 74 |
| 1 | 80-90 or 201-242 | 10 | 256 and 257 |
| 1 | 80-90 or 201-242 | 11 | 70 |
| 1 | 80-90 or 201-242 | 11 | 71 |
| 1 | 80-90 or 201-242 | 11 | 72 |
| 1 | 80-90 or 201-242 | 11 | 73 |
| 1 | 80-90 or 201-242 | 11 | 74 |
| 1 | 80-90 or 201-242 | 11 | 256 and 257 |
| 1 | 80-90 or 201-242 | 12 | 70 |
| 1 | 80-90 or 201-242 | 12 | 71 |
| 1 | 80-90 or 201-242 | 12 | 72 |
| 1 | 80-90 or 201-242 | 12 | 73 |
| 1 | 80-90 or 201-242 | 12 | 74 |
| 1 | 80-90 or 201-242 | 12 | 256 and 257 |
| 1 | 80-90 or 201-242 | 13 | 70 |
| 1 | 80-90 or 201-242 | 13 | 71 |
| 1 | 80-90 or 201-242 | 13 | 72 |
| 1 | 80-90 or 201-242 | 13 | 73 |
| 1 | 80-90 or 201-242 | 13 | 74 |
| 1 | 80-90 or 201-242 | 13 | 256 and 257 |
| 1 | 80-90 or 201-242 | 14 | 70 |
| 1 | 80-90 or 201-242 | 14 | 71 |
| 1 | 80-90 or 201-242 | 14 | 72 |
| 1 | 80-90 or 201-242 | 14 | 73 |
| 1 | 80-90 or 201-242 | 14 | 74 |
| 1 | 80-90 or 201-242 | 14 | 256 and 257 |
| 1 | 80-90 or 201-242 | 15 | 70 |
| 1 | 80-90 or 201-242 | 15 | 71 |
| 1 | 80-90 or 201-242 | 15 | 72 |
| 1 | 80-90 or 201-242 | 15 | 73 |
| 1 | 80-90 or 201-242 | 15 | 74 |
| 1 | 80-90 or 201-242 | 15 | 256 and 257 |
| 1 | 80-90 or 201-242 | 16 | 70 |
| 1 | 80-90 or 201-242 | 16 | 71 |
| 1 | 80-90 or 201-242 | 16 | 72 |
| 1 | 80-90 or 201-242 | 16 | 73 |
| 1 | 80-90 or 201-242 | 16 | 74 |
| 1 | 80-90 or 201-242 | 16 | 256 and 257 |
| 1 | 80-90 or 201-242 | 20 | 70 |
| 1 | 80-90 or 201-242 | 20 | 71 |
| 1 | 80-90 or 201-242 | 20 | 72 |
| 1 | 80-90 or 201-242 | 20 | 73 |
| 1 | 80-90 or 201-242 | 20 | 74 |
| 1 | 80-90 or 201-242 | 20 | 256 and 257 |
| 1 | 80-90 or 201-242 | 21 | 70 |
| 1 | 80-90 or 201-242 | 21 | 71 |
| 1 | 80-90 or 201-242 | 21 | 72 |
| 1 | 80-90 or 201-242 | 21 | 73 |
| 1 | 80-90 or 201-242 | 21 | 74 |
| 1 | 80-90 or 201-242 | 21 | 256 and 257 |
| 1 | 80-90 or 201-242 | 22 | 70 |
| 1 | 80-90 or 201-242 | 22 | 71 |
| 1 | 80-90 or 201-242 | 22 | 72 |
| 1 | 80-90 or 201-242 | 22 | 73 |
| 1 | 80-90 or 201-242 | 22 | 74 |
| 1 | 80-90 or 201-242 | 22 | 256 and 257 |
| 1 | 80-90 or 201-242 | 23 | 70 |
| 1 | 80-90 or 201-242 | 23 | 71 |
| 1 | 80-90 or 201-242 | 23 | 72 |
| 1 | 80-90 or 201-242 | 23 | 73 |
| 1 | 80-90 or 201-242 | 23 | 74 |
| 1 | 80-90 or 201-242 | 23 | 256 and 257 |
| 1 | 80-90 or 201-242 | 24 | 70 |
| 1 | 80-90 or 201-242 | 24 | 71 |
| 1 | 80-90 or 201-242 | 24 | 72 |
| 1 | 80-90 or 201-242 | 24 | 73 |
| 1 | 80-90 or 201-242 | 24 | 74 |
| 1 | 80-90 or 201-242 | 24 | 256 and 257 |
| 1 | 80-90 or 201-242 | 25 | 70 |
| 1 | 80-90 or 201-242 | 25 | 71 |
| 1 | 80-90 or 201-242 | 25 | 72 |
| 1 | 80-90 or 201-242 | 25 | 73 |
| 1 | 80-90 or 201-242 | 25 | 74 |
| 1 | 80-90 or 201-242 | 25 | 256 and 257 |
| 1 | 80-90 or 201-242 | 26 | 70 |
| 1 | 80-90 or 201-242 | 26 | 71 |
| 1 | 80-90 or 201-242 | 26 | 72 |
| 1 | 80-90 or 201-242 | 26 | 73 |
| 1 | 80-90 or 201-242 | 26 | 74 |
| 1 | 80-90 or 201-242 | 26 | 256 and 257 |
| 1 | 80-90 or 201-242 | 27 | 70 |
| 1 | 80-90 or 201-242 | 27 | 71 |
| 1 | 80-90 or 201-242 | 27 | 72 |
| 1 | 80-90 or 201-242 | 27 | 73 |
| 1 | 80-90 or 201-242 | 27 | 74 |
| 1 | 80-90 or 201-242 | 27 | 256 and 257 |
| 1 | 80-90 or 201-242 | 28 | 70 |
| 1 | 80-90 or 201-242 | 28 | 71 |
| 1 | 80-90 or 201-242 | 28 | 72 |
| 1 | 80-90 or 201-242 | 28 | 73 |
| 1 | 80-90 or 201-242 | 28 | 74 |
| 1 | 80-90 or 201-242 | 28 | 256 and 257 |
| 1 | 80-90 or 201-242 | 29 | 70 |
| 1 | 80-90 or 201-242 | 29 | 71 |
| 1 | 80-90 or 201-242 | 29 | 72 |
| 1 | 80-90 or 201-242 | 29 | 73 |
| 1 | 80-90 or 201-242 | 29 | 74 |
| 1 | 80-90 or 201-242 | 29 | 256 and 257 |
| 1 | 80-90 or 201-242 | 30 | 70 |
| 1 | 80-90 or 201-242 | 30 | 71 |
| 1 | 80-90 or 201-242 | 30 | 72 |
| 1 | 80-90 or 201-242 | 30 | 73 |
| 1 | 80-90 or 201-242 | 30 | 74 |
| 1 | 80-90 or 201-242 | 30 | 256 and 257 |
| 1 | 80-90 or 201-242 | 31 | 70 |
| 1 | 80-90 or 201-242 | 31 | 71 |
| 1 | 80-90 or 201-242 | 31 | 72 |
| 1 | 80-90 or 201-242 | 31 | 73 |
| 1 | 80-90 or 201-242 | 31 | 74 |
| 1 | 80-90 or 201-242 | 31 | 256 and 257 |
| 1 | 80-90 or 201-242 | 32 and 33 | 70 |
| 1 | 80-90 or 201-242 | 32 and 33 | 71 |
| 1 | 80-90 or 201-242 | 32 and 33 | 72 |
| 1 | 80-90 or 201-242 | 32 and 33 | 73 |
| 1 | 80-90 or 201-242 | 32 and 33 | 74 |
| 1 | 80-90 or 201-242 | 32 and 33 | 256 and 257 |
| 1 | 80-90 or 201-242 | 40 | 70 |
| 1 | 80-90 or 201-242 | 40 | 71 |
| 1 | 80-90 or 201-242 | 40 | 72 |
| 1 | 80-90 or 201-242 | 40 | 73 |
| 1 | 80-90 or 201-242 | 40 | 74 |
| 1 | 80-90 or 201-242 | 40 | 256 and 257 |
| 1 | 80-90 or 201-242 | 41 | 70 |
| 1 | 80-90 or 201-242 | 41 | 71 |
| 1 | 80-90 or 201-242 | 41 | 72 |
| 1 | 80-90 or 201-242 | 41 | 73 |
| 1 | 80-90 or 201-242 | 41 | 74 |
| 1 | 80-90 or 201-242 | 41 | 256 and 257 |
| 1 | 80-90 or 201-242 | 42 | 70 |
| 1 | 80-90 or 201-242 | 42 | 71 |
| 1 | 80-90 or 201-242 | 42 | 72 |
| 1 | 80-90 or 201-242 | 42 | 73 |
| 1 | 80-90 or 201-242 | 42 | 74 |
| 1 | 80-90 or 201-242 | 42 | 256 and 257 |
| 1 | 80-90 or 201-242 | 43 | 70 |
| 1 | 80-90 or 201-242 | 43 | 71 |
| 1 | 80-90 or 201-242 | 43 | 72 |
| 1 | 80-90 or 201-242 | 43 | 73 |
| 1 | 80-90 or 201-242 | 43 | 74 |
| 1 | 80-90 or 201-242 | 43 | 256 and 257 |
| 1 | 80-90 or 201-242 | 44 | 70 |
| 1 | 80-90 or 201-242 | 44 | 71 |
| 1 | 80-90 or 201-242 | 44 | 72 |
| 1 | 80-90 or 201-242 | 44 | 73 |
| 1 | 80-90 or 201-242 | 44 | 74 |
| 1 | 80-90 or 201-242 | 44 | 256 and 257 |
| 1 | 80-90 or 201-242 | 45 | 70 |
| 1 | 80-90 or 201-242 | 45 | 71 |
| 1 | 80-90 or 201-242 | 45 | 72 |
| 1 | 80-90 or 201-242 | 45 | 73 |
| 1 | 80-90 or 201-242 | 45 | 74 |
| 1 | 80-90 or 201-242 | 45 | 256 and 257 |
| 1 | 80-90 or 201-242 | 46 | 70 |
| 1 | 80-90 or 201-242 | 46 | 71 |
| 1 | 80-90 or 201-242 | 46 | 72 |
| 1 | 80-90 or 201-242 | 46 | 73 |
| 1 | 80-90 or 201-242 | 46 | 74 |
| 1 | 80-90 or 201-242 | 46 | 256 and 257 |
| 1 | 80-90 or 201-242 | 47 | 70 |
| 1 | 80-90 or 201-242 | 47 | 71 |
| 1 | 80-90 or 201-242 | 47 | 72 |
| 1 | 80-90 or 201-242 | 47 | 73 |
| 1 | 80-90 or 201-242 | 47 | 74 |
| 1 | 80-90 or 201-242 | 47 | 256 and 257 |
| 1 | 80-90 or 201-242 | 48 | 70 |
| 1 | 80-90 or 201-242 | 48 | 71 |
| 1 | 80-90 or 201-242 | 48 | 72 |
| 1 | 80-90 or 201-242 | 48 | 73 |
| 1 | 80-90 or 201-242 | 48 | 74 |
| 1 | 80-90 or 201-242 | 48 | 256 and 257 |
| 1 | 80-90 or 201-242 | 49 | 70 |
| 1 | 80-90 or 201-242 | 49 | 71 |
| 1 | 80-90 or 201-242 | 49 | 72 |
| 1 | 80-90 or 201-242 | 49 | 73 |
| 1 | 80-90 or 201-242 | 49 | 74 |
| 1 | 80-90 or 201-242 | 49 | 256 and 257 |
| 1 | 80-90 or 201-242 | 50 | 70 |
| 1 | 80-90 or 201-242 | 50 | 71 |
| 1 | 80-90 or 201-242 | 50 | 72 |
| 1 | 80-90 or 201-242 | 50 | 73 |
| 1 | 80-90 or 201-242 | 50 | 74 |
| 1 | 80-90 or 201-242 | 50 | 256 and 257 |
| 1 | 80-90 or 201-242 | 51 | 70 |
| 1 | 80-90 or 201-242 | 51 | 71 |
| 1 | 80-90 or 201-242 | 51 | 72 |
| 1 | 80-90 or 201-242 | 51 | 73 |
| 1 | 80-90 or 201-242 | 51 | 74 |
| 1 | 80-90 or 201-242 | 51 | 256 and 257 |
| 1 | 80-90 or 201-242 | 247 | 70 |
| 1 | 80-90 or 201-242 | 247 | 71 |
| 1 | 80-90 or 201-242 | 247 | 72 |
| 1 | 80-90 or 201-242 | 247 | 73 |
| 1 | 80-90 or 201-242 | 247 | 74 |
| 1 | 80-90 or 201-242 | 247 | 256 and 257 |
| 1 | 80-90 or 201-242 | 248 and 249 | 70 |
| 1 | 80-90 or 201-242 | 248 and 249 | 71 |
| 1 | 80-90 or 201-242 | 248 and 249 | 72 |
| 1 | 80-90 or 201-242 | 248 and 249 | 73 |
| 1 | 80-90 or 201-242 | 248 and 249 | 74 |
| 1 | 80-90 or 201-242 | 248 and 249 | 256 and 257 |
| 2 | 80-90 or 201-242 | 10 | |
| 2 | 80-90 or 201-242 | 11 | |
| 2 | 80-90 or 201-242 | 12 | |
| 2 | 80-90 or 201-242 | 13 | |
| 2 | 80-90 or 201-242 | 14 | |
| 2 | 80-90 or 201-242 | 15 | |
| 2 | 80-90 or 201-242 | 16 | |
| 2 | 80-90 or 201-242 | 20 | |
| 2 | 80-90 or 201-242 | 21 | |
| 2 | 80-90 or 201-242 | 22 | |
| 2 | 80-90 or 201-242 | 23 | |
| 2 | 80-90 or 201-242 | 24 | |
| 2 | 80-90 or 201-242 | 25 | |
| 2 | 80-90 or 201-242 | 26 | |
| 2 | 80-90 or 201-242 | 27 | |
| 2 | 80-90 or 201-242 | 28 | |
| 2 | 80-90 or 201-242 | 29 | |
| 2 | 80-90 or 201-242 | 30 | |
| 2 | 80-90 or 201-242 | 31 | |
| 2 | 80-90 or 201-242 | 32 and 33 | |
| 2 | 80-90 or 201-242 | 40 | |
| 2 | 80-90 or 201-242 | 41 | |
| 2 | 80-90 or 201-242 | 42 | |
| 2 | 80-90 or 201-242 | 43 | |
| 2 | 80-90 or 201-242 | 44 | |
| 2 | 80-90 or 201-242 | 45 | |
| 2 | 80-90 or 201-242 | 46 | |
| 2 | 80-90 or 201-242 | 47 | |
| 2 | 80-90 or 201-242 | 48 | |
| 2 | 80-90 or 201-242 | 49 | |
| 2 | 80-90 or 201-242 | 50 | |
| 2 | 80-90 or 201-242 | 51 | |
| 2 | 80-90 or 201-242 | 247 | |
| 2 | 80-90 or 201-242 | 248 and 249 | |
| 2 | 80-90 or 201-242 | 10 | 70 |
| 2 | 80-90 or 201-242 | 10 | 71 |
| 2 | 80-90 or 201-242 | 10 | 72 |
| 2 | 80-90 or 201-242 | 10 | 73 |
| 2 | 80-90 or 201-242 | 10 | 74 |
| 2 | 80-90 or 201-242 | 10 | 256 and 257 |
| 2 | 80-90 or 201-242 | 11 | 70 |
| 2 | 80-90 or 201-242 | 11 | 71 |
| 2 | 80-90 or 201-242 | 11 | 72 |
| 2 | 80-90 or 201-242 | 11 | 73 |
| 2 | 80-90 or 201-242 | 11 | 74 |
| 2 | 80-90 or 201-242 | 11 | 256 and 257 |
| 2 | 80-90 or 201-242 | 12 | 70 |
| 2 | 80-90 or 201-242 | 12 | 71 |
| 2 | 80-90 or 201-242 | 12 | 72 |
| 2 | 80-90 or 201-242 | 12 | 73 |
| 2 | 80-90 or 201-242 | 12 | 74 |
| 2 | 80-90 or 201-242 | 12 | 256 and 257 |
| 2 | 80-90 or 201-242 | 13 | 70 |
| 2 | 80-90 or 201-242 | 13 | 71 |
| 2 | 80-90 or 201-242 | 13 | 72 |
| 2 | 80-90 or 201-242 | 13 | 73 |
| 2 | 80-90 or 201-242 | 13 | 74 |
| 2 | 80-90 or 201-242 | 13 | 256 and 257 |
| 2 | 80-90 or 201-242 | 14 | 70 |
| 2 | 80-90 or 201-242 | 14 | 71 |
| 2 | 80-90 or 201-242 | 14 | 72 |
| 2 | 80-90 or 201-242 | 14 | 73 |
| 2 | 80-90 or 201-242 | 14 | 74 |
| 2 | 80-90 or 201-242 | 14 | 256 and 257 |
| 2 | 80-90 or 201-242 | 15 | 70 |
| 2 | 80-90 or 201-242 | 15 | 71 |
| 2 | 80-90 or 201-242 | 15 | 72 |
| 2 | 80-90 or 201-242 | 15 | 73 |
| 2 | 80-90 or 201-242 | 15 | 74 |
| 2 | 80-90 or 201-242 | 15 | 256 and 257 |
| 2 | 80-90 or 201-242 | 16 | 70 |
| 2 | 80-90 or 201-242 | 16 | 71 |
| 2 | 80-90 or 201-242 | 16 | 72 |
| 2 | 80-90 or 201-242 | 16 | 73 |
| 2 | 80-90 or 201-242 | 16 | 74 |
| 2 | 80-90 or 201-242 | 16 | 256 and 257 |
| 2 | 80-90 or 201-242 | 20 | 70 |
| 2 | 80-90 or 201-242 | 20 | 71 |
| 2 | 80-90 or 201-242 | 20 | 72 |
| 2 | 80-90 or 201-242 | 20 | 73 |
| 2 | 80-90 or 201-242 | 20 | 74 |
| 2 | 80-90 or 201-242 | 20 | 256 and 257 |
| 2 | 80-90 or 201-242 | 21 | 70 |
| 2 | 80-90 or 201-242 | 21 | 71 |
| 2 | 80-90 or 201-242 | 21 | 72 |
| 2 | 80-90 or 201-242 | 21 | 73 |
| 2 | 80-90 or 201-242 | 21 | 74 |
| 2 | 80-90 or 201-242 | 21 | 256 and 257 |
| 2 | 80-90 or 201-242 | 22 | 70 |
| 2 | 80-90 or 201-242 | 22 | 71 |
| 2 | 80-90 or 201-242 | 22 | 72 |
| 2 | 80-90 or 201-242 | 22 | 73 |
| 2 | 80-90 or 201-242 | 22 | 74 |
| 2 | 80-90 or 201-242 | 22 | 256 and 257 |
| 2 | 80-90 or 201-242 | 23 | 70 |
| 2 | 80-90 or 201-242 | 23 | 71 |
| 2 | 80-90 or 201-242 | 23 | 72 |
| 2 | 80-90 or 201-242 | 23 | 73 |
| 2 | 80-90 or 201-242 | 23 | 74 |
| 2 | 80-90 or 201-242 | 23 | 256 and 257 |
| 2 | 80-90 or 201-242 | 24 | 70 |
| 2 | 80-90 or 201-242 | 24 | 71 |
| 2 | 80-90 or 201-242 | 24 | 72 |
| 2 | 80-90 or 201-242 | 24 | 73 |
| 2 | 80-90 or 201-242 | 24 | 74 |
| 2 | 80-90 or 201-242 | 24 | 256 and 257 |
| 2 | 80-90 or 201-242 | 25 | 70 |
| 2 | 80-90 or 201-242 | 25 | 71 |
| 2 | 80-90 or 201-242 | 25 | 72 |
| 2 | 80-90 or 201-242 | 25 | 73 |
| 2 | 80-90 or 201-242 | 25 | 74 |
| 2 | 80-90 or 201-242 | 25 | 256 and 257 |
| 2 | 80-90 or 201-242 | 26 | 70 |
| 2 | 80-90 or 201-242 | 26 | 71 |
| 2 | 80-90 or 201-242 | 26 | 72 |
| 2 | 80-90 or 201-242 | 26 | 73 |
| 2 | 80-90 or 201-242 | 26 | 74 |
| 2 | 80-90 or 201-242 | 26 | 256 and 257 |
| 2 | 80-90 or 201-242 | 27 | 70 |
| 2 | 80-90 or 201-242 | 27 | 71 |
| 2 | 80-90 or 201-242 | 27 | 72 |
| 2 | 80-90 or 201-242 | 27 | 73 |
| 2 | 80-90 or 201-242 | 27 | 74 |
| 2 | 80-90 or 201-242 | 27 | 256 and 257 |
| 2 | 80-90 or 201-242 | 28 | 70 |
| 2 | 80-90 or 201-242 | 28 | 71 |
| 2 | 80-90 or 201-242 | 28 | 72 |
| 2 | 80-90 or 201-242 | 28 | 73 |
| 2 | 80-90 or 201-242 | 28 | 74 |
| 2 | 80-90 or 201-242 | 28 | 256 and 257 |
| 2 | 80-90 or 201-242 | 29 | 70 |
| 2 | 80-90 or 201-242 | 29 | 71 |
| 2 | 80-90 or 201-242 | 29 | 72 |
| 2 | 80-90 or 201-242 | 29 | 73 |
| 2 | 80-90 or 201-242 | 29 | 74 |
| 2 | 80-90 or 201-242 | 29 | 256 and 257 |
| 2 | 80-90 or 201-242 | 30 | 70 |
| 2 | 80-90 or 201-242 | 30 | 71 |
| 2 | 80-90 or 201-242 | 30 | 72 |
| 2 | 80-90 or 201-242 | 30 | 73 |
| 2 | 80-90 or 201-242 | 30 | 74 |
| 2 | 80-90 or 201-242 | 30 | 256 and 257 |
| 2 | 80-90 or 201-242 | 31 | 70 |
| 2 | 80-90 or 201-242 | 31 | 71 |
| 2 | 80-90 or 201-242 | 31 | 72 |
| 2 | 80-90 or 201-242 | 31 | 73 |
| 2 | 80-90 or 201-242 | 31 | 74 |
| 2 | 80-90 or 201-242 | 31 | 256 and 257 |
| 2 | 80-90 or 201-242 | 32 and 33 | 70 |
| 2 | 80-90 or 201-242 | 32 and 33 | 71 |
| 2 | 80-90 or 201-242 | 32 and 33 | 72 |
| 2 | 80-90 or 201-242 | 32 and 33 | 73 |
| 2 | 80-90 or 201-242 | 32 and 33 | 74 |
| 2 | 80-90 or 201-242 | 32 and 33 | 256 and 257 |
| 2 | 80-90 or 201-242 | 40 | 70 |
| 2 | 80-90 or 201-242 | 40 | 71 |
| 2 | 80-90 or 201-242 | 40 | 72 |
| 2 | 80-90 or 201-242 | 40 | 73 |
| 2 | 80-90 or 201-242 | 40 | 74 |
| 2 | 80-90 or 201-242 | 40 | 256 and 257 |
| 2 | 80-90 or 201-242 | 41 | 70 |
| 2 | 80-90 or 201-242 | 41 | 71 |
| 2 | 80-90 or 201-242 | 41 | 72 |
| 2 | 80-90 or 201-242 | 41 | 73 |
| 2 | 80-90 or 201-242 | 41 | 74 |
| 2 | 80-90 or 201-242 | 41 | 256 and 257 |
| 2 | 80-90 or 201-242 | 42 | 70 |
| 2 | 80-90 or 201-242 | 42 | 71 |
| 2 | 80-90 or 201-242 | 42 | 72 |
| 2 | 80-90 or 201-242 | 42 | 73 |
| 2 | 80-90 or 201-242 | 42 | 74 |
| 2 | 80-90 or 201-242 | 42 | 256 and 257 |
| 2 | 80-90 or 201-242 | 43 | 70 |
| 2 | 80-90 or 201-242 | 43 | 71 |
| 2 | 80-90 or 201-242 | 43 | 72 |
| 2 | 80-90 or 201-242 | 43 | 73 |
| 2 | 80-90 or 201-242 | 43 | 74 |
| 2 | 80-90 or 201-242 | 43 | 256 and 257 |
| 2 | 80-90 or 201-242 | 44 | 70 |
| 2 | 80-90 or 201-242 | 44 | 71 |
| 2 | 80-90 or 201-242 | 44 | 72 |
| 2 | 80-90 or 201-242 | 44 | 73 |
| 2 | 80-90 or 201-242 | 44 | 74 |
| 2 | 80-90 or 201-242 | 44 | 256 and 257 |
| 2 | 80-90 or 201-242 | 45 | 70 |
| 2 | 80-90 or 201-242 | 45 | 71 |
| 2 | 80-90 or 201-242 | 45 | 72 |
| 2 | 80-90 or 201-242 | 45 | 73 |
| 2 | 80-90 or 201-242 | 45 | 74 |
| 2 | 80-90 or 201-242 | 45 | 256 and 257 |
| 2 | 80-90 or 201-242 | 46 | 70 |
| 2 | 80-90 or 201-242 | 46 | 71 |
| 2 | 80-90 or 201-242 | 46 | 72 |
| 2 | 80-90 or 201-242 | 46 | 73 |
| 2 | 80-90 or 201-242 | 46 | 74 |
| 2 | 80-90 or 201-242 | 46 | 256 and 257 |
| 2 | 80-90 or 201-242 | 47 | 70 |
| 2 | 80-90 or 201-242 | 47 | 71 |
| 2 | 80-90 or 201-242 | 47 | 72 |
| 2 | 80-90 or 201-242 | 47 | 73 |
| 2 | 80-90 or 201-242 | 47 | 74 |
| 2 | 80-90 or 201-242 | 47 | 256 and 257 |
| 2 | 80-90 or 201-242 | 48 | 70 |
| 2 | 80-90 or 201-242 | 48 | 71 |
| 2 | 80-90 or 201-242 | 48 | 72 |
| 2 | 80-90 or 201-242 | 48 | 73 |
| 2 | 80-90 or 201-242 | 48 | 74 |
| 2 | 80-90 or 201-242 | 48 | 256 and 257 |
| 2 | 80-90 or 201-242 | 49 | 70 |
| 2 | 80-90 or 201-242 | 49 | 71 |
| 2 | 80-90 or 201-242 | 49 | 72 |
| 2 | 80-90 or 201-242 | 49 | 73 |
| 2 | 80-90 or 201-242 | 49 | 74 |
| 2 | 80-90 or 201-242 | 49 | 256 and 257 |
| 2 | 80-90 or 201-242 | 50 | 70 |
| 2 | 80-90 or 201-242 | 50 | 71 |
| 2 | 80-90 or 201-242 | 50 | 72 |
| 2 | 80-90 or 201-242 | 50 | 73 |
| 2 | 80-90 or 201-242 | 50 | 74 |
| 2 | 80-90 or 201-242 | 50 | 256 and 257 |
| 2 | 80-90 or 201-242 | 51 | 70 |
| 2 | 80-90 or 201-242 | 51 | 71 |
| 2 | 80-90 or 201-242 | 51 | 72 |
| 2 | 80-90 or 201-242 | 51 | 73 |
| 2 | 80-90 or 201-242 | 51 | 74 |
| 2 | 80-90 or 201-242 | 51 | 256 and 257 |
| 2 | 80-90 or 201-242 | 247 | 70 |
| 2 | 80-90 or 201-242 | 247 | 71 |
| 2 | 80-90 or 201-242 | 247 | 72 |
| 2 | 80-90 or 201-242 | 247 | 73 |
| 2 | 80-90 or 201-242 | 247 | 74 |
| 2 | 80-90 or 201-242 | 247 | 256 and 257 |
| 2 | 80-90 or 201-242 | 248 and 249 | 70 |
| 2 | 80-90 or 201-242 | 248 and 249 | 71 |
| 2 | 80-90 or 201-242 | 248 and 249 | 72 |
| 2 | 80-90 or 201-242 | 248 and 249 | 73 |
| 2 | 80-90 or 201-242 | 248 and 249 | 74 |
| 2 | 80-90 or 201-242 | 248 and 249 | 256 and 257 |

Additionally, any one of the cytokine prodrugs described in Table 2 may comprise a consensus sequence according to any one of SEQ ID NOs: 91-94 in place of the listed protease-cleavable sequences.

Also encompassed by this disclosure are cytokine prodrugs comprising a sequence with at least 80, 85, 90, 95, 97, 98, or 99 percent identity to the sequence of any one of the cytokine prodrugs described above.

In some embodiments, the cytokine prodrug comprises a sequence with at least 80, 85, 90, 95, 97, 98, or 99 percent identity to the sequence of any one of SEQ ID NOs: 100-111. In some embodiments, the cytokine prodrug comprises the sequence of any one of SEQ ID NOs: 100-111.

Exemplary constructs are shown in Table 3. In some embodiments, the cytokine prodrug comprises elements as set forth in any of the constructs of Table 3.

TABLE 3
Exemplary cytokine prodrug constructs. See following paragraph for SEQ ID NOs.

| Name | Features |
|---|---|
| Construct A | mIL2- 2x(SG4) - MMPcs1 - 2x(G4S) - IL2Ralpha - 6His |
| Construct B | m IL2-2x(SG4) - MMPcs1 - 2x (G4S) - IL2Ralpha - mIgG1 Fc |
| Construct C | m IL2(C140S)- 2x(SG4) - MMPcs1 - 2x(G4S) - IL2Ralpha - mIgG1 |
| | Fc(T252M)- 6xHIS |
| Construct D | m IL2(C140S)- 2x(SG4) - MMPcs1 - 2x(G4S) - soluble IL2Ralpha - |
| | mIgG1 Fc(T252M)- 6xHIS |
| Construct E | Hu IL2(C125S)- 2x(SG4) - MMPcs1 - 2x(G4S) - IL2Ralpha - hu IgGl Fc - 6xHIS |
| Construct F | hIL2(C125S)-2x(SG4)-MMPcs1-2x(G4S)-chimeric IL2Ra(sushi |
| | mouse)-hIgG1 Fc |
| Construct G | hIL2(C125S)-2x(SG4)-MMPcs1-2x(G4S)-hIL2Ra(1-219)-GSGGGG- |
| | hIgG1 Fc |
| Construct H | hIL2(C125S)-2x(SG4)-MMPcs1-2x(G4S)-hIL2Ra(1-178)-GSGGGG- |
| | hIgG1 Fc |
| Construct I | hIL2(C125S)-2x(SG4)-MMPcs1-2x(G4S)-hIL2Ra(M25I)-hIgG1 Fc- |
| | His |
| Construct J | hIL2(C125S)-2x(SG4)-MMPcs1-2x(G4S)-hIL2Ra(L42V)-hIgG1 Fc- |
| | His |
| Construct K | hIL2(C125S)-2x(SG4)-MMPcs1-2x(G4S)-hIL2Ra(D4L 5LY)-hIgG1 |
| | Fc-His |
| Construct L | hIL2(C125S)-2x(SG4)-MMPcs1-2x(G4S)-hIL2Ra(SGSL₃₉₋₄₂ELV)- |
| | hIgG1 Fc-His |
| Construct M | hIL2(C125S)-2x(SG4)-MMPcs1-2x(G4S)-hIL2Ra(1-192)-hIgG1 Fc |
| Construct N | hIL2(C125S)-2x(SG4)-MMPcs1-2x(G4S)-hIL2Ra(1-192)-GSGGGG- |
| | hIgG1 Fc |
| Construct O | hIL2(C125S)-2x(SG4)-MMPcs1-2x(G4S)-hIL2Ra(1-192/M25I)- |
| | GSGGGG-hIgG1 Fc |
| Construct P | hIL2(C125S)-2x(SG4)-MMPcs1-2x(G4S)-hIL2Ra(1-192/L42V)- |
| | GSGGGG-hIgG1 Fc |
| Construct Q | hIL2(C125S)-2x(SG4)-MMPcs1-2x(G4S)-hIL2Ra(1-192/D4L D5Y)- |
| | GSGGGG-hIgG1 Fc |

The C-terminal “His” represents a hexahistidine tag, which is optional and may be omitted in some embodiments. The sequences of Construct A, Construct B, Construct C, Construct D, and Construct E are SEQ ID NOs: 100, 101, 102, 104, and 106, respectively. The sequence of hIL2(C125S) is SEQ ID NO: 2. The sequence of 2×(SG4) is SGGGGSGGGG (SEQ ID NO: 243). The sequence of MMPcs1 is GPLGVRG (SEQ ID NO: 80). The sequence of 2×(G4S) is GGGGSGGGGS (SEQ ID NO: 244). The sequence of hIgG1 Fc is SEQ ID NO: 70. The sequence of chimeric IL2Ra(sushi mouse) is SEQ ID NO: 17. The sequence of hIL2Ra(1-219) is SEQ ID NO: 10. GSGGGG is SEQ ID NO: 245. The sequences of hIL2Ra(1-178), hIL2Ra(M25I), hIL2Ra(L42V), hIL2Ra(SGSL₃₉₋₄₂ELV), and hIL2Ra(D4L 5LY) are SEQ ID NOs: 44, 12, 13, 24, and 23, respectively. The sequences of hIL2Ra(1-192), hIL2Ra(1-192/M25I), hIL2Ra(1-192/L42V), and hIL2Ra(1-192/D4L D5 Y) are SEQ ID NOs: 25, 26, 27, and 28, respectively.

### Pharmaceutical Formulations

Pharmaceutical formulations of a cytokine prodrug as described herein may be prepared by mixing such cytokine prodrug having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

The formulations to be used for in vivo administration are generally sterile. Sterility may be readily accomplished, e.g., by filtration through sterile filtration membranes.

### Uses

In some embodiments, any one or more of the cytokine prodrugs, compositions, or pharmaceutical formulations described herein is for use in preparing a medicament for treating or preventing a disease or disorder in a subject. In some embodiments, any one or more of the cytokine prodrugs, compositions, or pharmaceutical formulations described herein is for use in a method of creating a cytokine gradient in a subject, comprising administering the protease-activated pro-cytokine or pharmaceutical composition to a subject, wherein the subject comprises a site having an abnormally high level of a protease that cleaves the protease-cleavable polypeptide sequence, optionally wherein the site comprises a cancer. In some embodiments, the abnormally high level is higher than the level of the protease in a healthy tissue of the same type as the site with the abnormally high level (e.g., in the subject being treated or in a healthy subject). In some embodiments, the abnormally high level is higher than the average level of the protease in soft tissue.

In some embodiments, a method of treating or preventing a disease or disorder in subject is provided, comprising administering to a subject any of the cytokine prodrugs or pharmaceutical compositions described herein. In some embodiments, the disease or disorder is a cancer, e.g., a solid tumor. In some embodiments, the cancer is a melanoma, a colorectal cancer, a breast cancer, a pancreatic cancer, a lung cancer, a prostate cancer, an ovarian cancer, a cervical cancer, a gastric or gastrointestinal cancer, a lymphoma, a colon or colorectal cancer, an endometrial cancer, a thyroid cancer, or a bladder cancer. The cancer (e.g., any of the foregoing cancers) may have one or more of the following features: being PD-L1-positive; being metastatic; being unresectable; being mismatch repair defective (MMRd); and/or being microsatellite-instability high (MSI-H).

In some embodiments, a method of boosting T regulatory cells and/or reducing inflammation or autoimmune activity is provided comprising administering a cytokine prodrug to an area of interest, e.g., an area of inflammation. The cytokine prodrug for use in such methods may comprise an IL-2 polypeptide sequence. In some embodiments, a method of treating an autoimmune and/or inflammatory disease is provided, comprising administering a cytokine prodrug to an area of interest, e.g., an area of inflammation or autoimmune activity. The cytokine prodrug for use in such methods may comprise an IL-2 polypeptide sequence. These methods take advantage of the ability of certain cytokines at relatively low levels to stimulate T regulatory cells, which can exert anti-inflammatory effects and reduce or suppress autoimmune activity.

The cytokine prodrugs in any of the foregoing methods and uses may be delivered to a subject using any suitable route of administration. In some embodiments, the cytokine prodrug is delivered parenterally. In some embodiments, the cytokine prodrug is delivered intravenously.

A cytokine prodrug provided herein can be used either alone or in combination with other agents in a therapy. For instance, a cytokine prodrug provided herein may be co-administered with at least one additional therapeutic agent.

Such combination therapies noted above encompass combined administration (where two or more therapeutic agents are included in the same or separate formulations), and separate administration, in which case, administration of the cytokine prodrug provided herein can occur prior to, simultaneously, and/or following, administration of the additional therapeutic agent and/or adjuvant.

Cytokine prodrugs would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

For the prevention or treatment of disease, the appropriate dosage of an cytokine prodrug (when used alone or in combination with one or more other additional therapeutic agents) will depend on the type of disease to be treated, the type of cytokine prodrug, the severity and course of the disease, whether the cytokine prodrug is administered for preventive or therapeutic purposes, previous therapy, the patient's clinical history and response to the antibody or immunoconjugate, and the discretion of the attending physician. The cytokine prodrug is suitably administered to the patient at one time or over a series of treatments.

### Nucleic Acids, Host Cells, and Production Methods

Cytokine prodrugs or precursors thereof may be produced using recombinant methods and compositions. In some embodiments, isolated nucleic acid encoding a cytokine prodrug described herein is provided. Such nucleic acid may encode an amino acid sequence comprising the cytokine polypeptide sequence, the linker, and the inhibitory polypeptide sequence, and any other polypeptide components of the cytokine prodrug that may be present. Exemplary nucleic acid sequences are provided in Table 1. In a further embodiment, one or more vectors (e.g., expression vectors) comprising such nucleic acid are provided. In a further embodiment, a host cell comprising such nucleic acid is provided. In some such embodiments, a host cell comprises (e.g., has been transformed with) a vector comprising a nucleic acid that encodes a cytokine prodrug according to the disclosure. In some embodiments, the host cell is eukaryotic, e.g. a Chinese Hamster Ovary (CHO) cell or lymphoid cell (e.g., Y0, NS0, Sp20 cell). In some embodiments, a method of making a cytokine prodrug disclosed herein is provided, wherein the method comprises culturing a host cell comprising a nucleic acid encoding the cytokine prodrug, as provided above, under conditions suitable for expression of the cytokine prodrug, and optionally recovering the antibody from the host cell (or host cell culture medium).

For recombinant production of a cytokine prodrug, nucleic acid encoding the cytokine prodrug, e.g., as described above, is prepared and/or isolated (e.g., following construction using synthetic and/or molecular cloning techniques) and inserted into one or more vectors for further cloning and/or expression in a host cell. Such nucleic acid may be readily prepared and/or isolated using known techniques.

Suitable host cells for cloning or expression of cytokine prodrug-encoding vectors include prokaryotic or eukaryotic cells described herein. For example, a cytokine prodrug may be produced in bacteria, in particular when glycosylation is not needed. For expression of polypeptides in bacteria, see, e.g., U.S. Pat. Nos. 5,648,237, 5,789,199, and 5,840,523. After expression, the cytokine prodrug may be isolated from the bacterial cell paste in a soluble fraction and can be further purified.

In addition to prokaryotes, eukaryotic microbes such as filamentous fungi or yeast are suitable cloning or expression hosts for cytokine prodrug-encoding vectors, including fungi and yeast strains whose glycosylation pathways have been “humanized,” resulting in the production of polypeptides with a partially or fully human glycosylation pattern. See Gerngross, Nat. Biotech. 22:1409-1414 (2004), and Li et al., Nat. Biotech. 24:210-215 (2006).

Suitable host cells for the expression of cytokine prodrugs are also derived from multicellular organisms (plants, invertebrates, and vertebrates). Examples of invertebrate cells include insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. “About” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

### EXAMPLES

The following examples are provided to illustrate certain disclosed embodiments and are not to be construed as limiting the scope of this disclosure in any way.

### Example 1: Construction of Mammalian Expression Vectors Encoding Fusion Proteins

Coding sequences for all protein domains including linker sequences were synthesized as an entire gene (Genscript, NJ). All synthetic genes were designed to contain a coding sequence for an N-terminal signal peptide (to facilitate protein secretion), a 5′ Kozak sequence, and unique restriction sites at the 5′ and 3′ ends. These genes were then directionally cloned into the mammalian expression vector pcDNA3.1 (Invitrogen, Carlsbad, Calif.). Examples of fusion protein constructs are listed in Table 3, above. Site directed mutagenesis was performed using standard molecular biology techniques and appropriate kit (GeneArt, Regensburg).

### Example 2: Expression and Purification of Fusion Proteins

### Transient Expression of Fusion Proteins

Two different CHO cell expression systems were used to produce fusion proteins (ExpiCHO-S™ and Freestyle CHO-S™, Life Technologies). Briefly, expression constructs were transiently transfected into CHO cells following manufacturer's protocol and using reagents provided in respective expression kits. Fusion proteins were then expressed and secreted into the cell culture supernatant. Samples were collected from the production cultures every day and cell density and viability are assessed. Protein expression titers and product integrity in cell culture supernatants were analyzed by SDS-PAGE to determine the optimal harvesting time. Cell culture supernatants were generally harvested between 4 and 12 days at culture viabilities of typically >75%. On day of harvest, cell culture supernatants were cleared by centrifugation and vacuum filtration before further use.

### Purification of Fusion Proteins

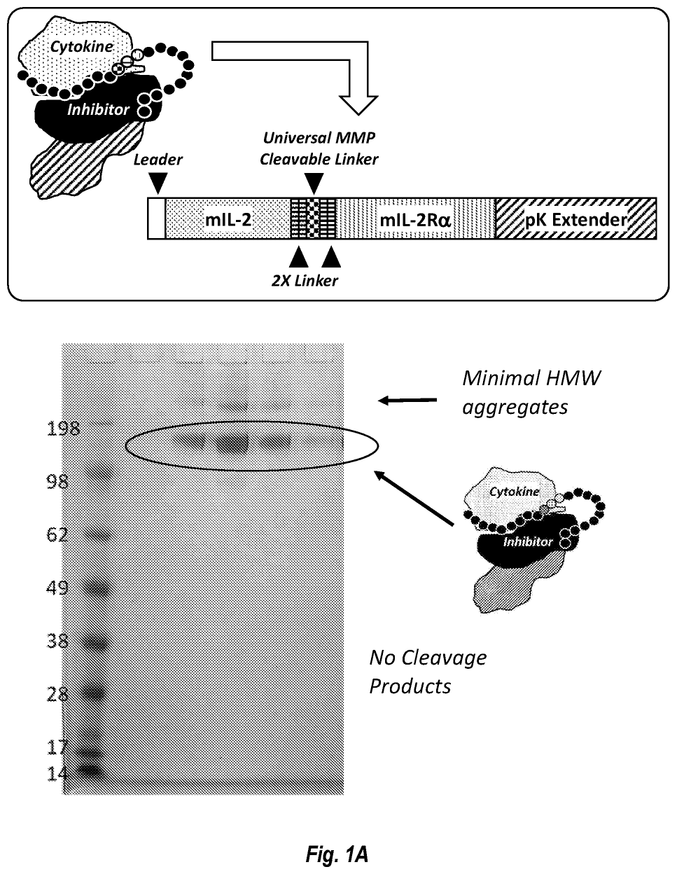

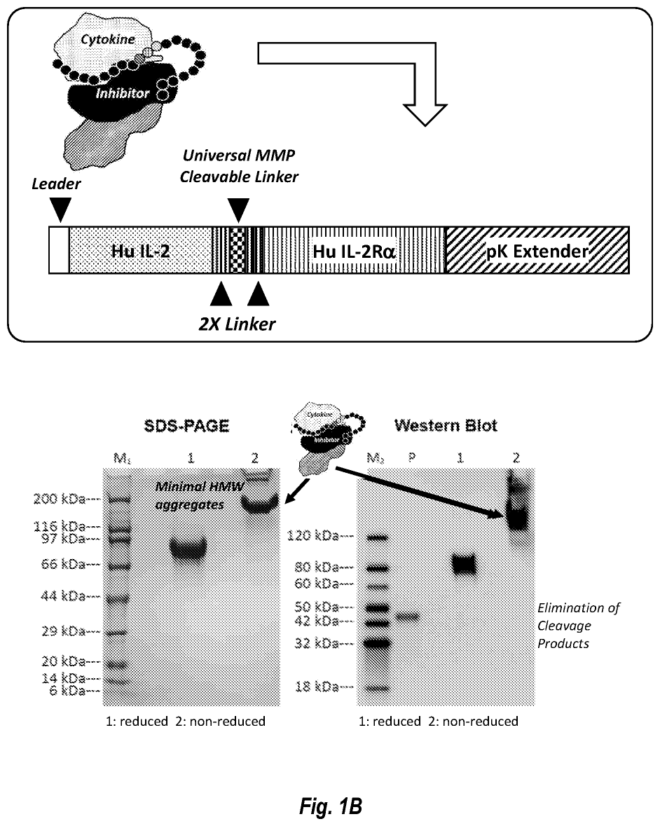

Fusion proteins were purified from CHO cell culture supernatants in either a one-step or two-step procedure. Briefly, Fc domain containing proteins were purified by Protein A affinity chromatography (HiTrap MabSelect SuRe, GE Healthcare). His-tagged proteins were first purified on a Nickel-agarose column (Ni-NTA Agarose, Qiagen), followed by anion ion exchange chromatography (HiTrap Capto Q ImpRes, Sigma). All purified samples were buffer-exchanged and concentrated by ultrafiltration to a typical concentration of >1 mg/mL. Purity and homogeneity (typically >90%) of final samples were assessed by SDS PAGE under reducing and non-reducing conditions, followed by immunoblotting using an anti-His or anti-Fc antibody. Purified proteins were aliquoted and stored at −80° C. until further use. FIG. 1 shows examples of successfully purified fusion proteins. See Table 3 for information regarding these constructs.

### Example 3: Cleavage of Fusion Protein by MMP Proteases

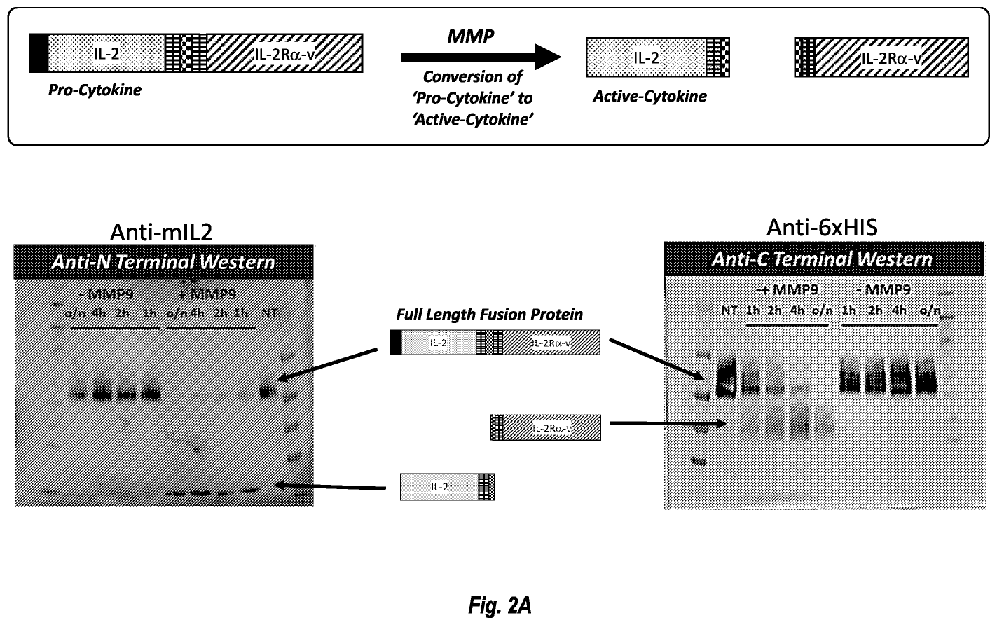

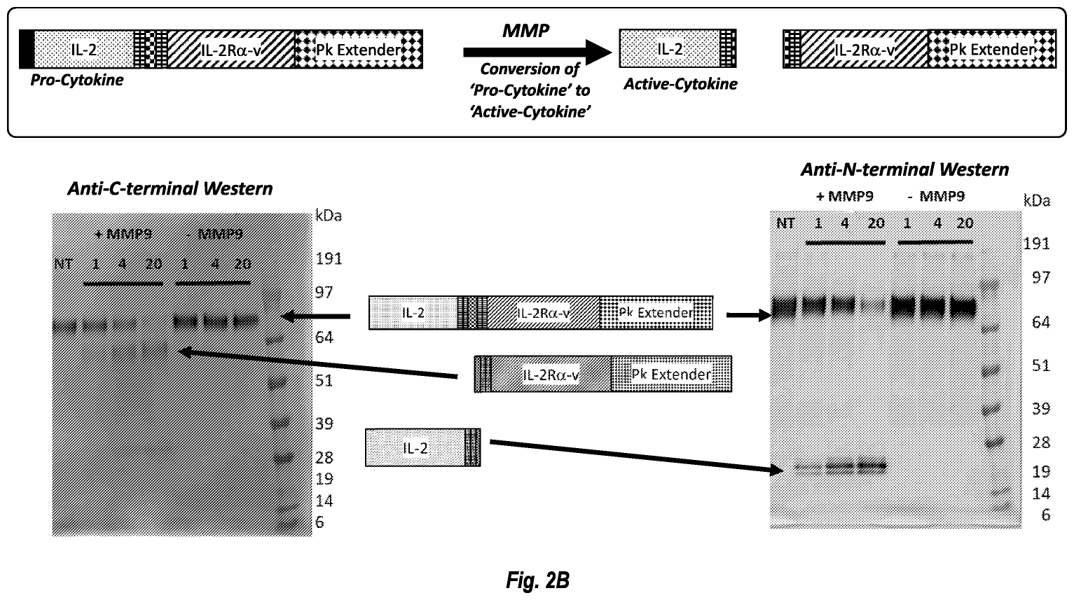

Recombinant MMP9 and/or MMP2 (R&D Systems) was first activated with p-aminophenylmercuric acetate and this activated protease or equivalent amount of activating solution without the protease was used to digest or mock-digest the fusion protein for 1 hr, 2 hr, 4 hr and overnight (18-22 hr) at 37 C. Cleavage assays are set up in TCNB buffer: 50 mM Tris, 10 mM CaCl2, 150 mM NaCl, 0.05% Brij-35 (w/v), pH 7.5. Digested protein was aliquoted and stored at −80° C. prior to testing. Aliquots of digests were subsequently analyzed by SDS-PAGE followed by Western blotting to evaluate the extent of cleavage. Digests were also assessed in functional assays such as CTLL-2 proliferation and HEK-Blue Interleukin reporter assays. As shown in FIGS. 2A-2C, essentially complete cleavage of the fusion proteins by MMP9 protease was seen after overnight incubation.

### Example 4: Detection of Mouse IL-2/IL-2Ra Fusion Proteins by ELISA

An ELISA assay was developed to detect and quantify fusion proteins comprising IL-2 and IL-2Ra moieties. Wells of a 96-well plate were coated overnight with 100 uL of a rat anti-mouse IL-2 monoclonal antibody (JES6-1A12; ThermoFisher) at 1 mg/ml in PBS. After washing, wells are blocked with TBS/0.05% Tween 20/1% BSA, then fusion proteins were added for 1 hr at room temperature. After washing, an anti-mouse IL-2Ra biotin-labelled detection antibody (BAF2438, R&D systems) was added and binding is detected using Ultra Strepavidin HRP (ThermoFisher). The ELISA plate was developed by adding the chromogenic tetramethylbenzidine substrate (Ultra TMB, ThermoFisher). The reaction is stopped by addition of 0.5M H2SO4 and the absorbance is read at 450-650 nm.

### Example 5: IL-2, IL-2Ra, 6×Histidine and Fc Immunoblot Analyses

Untreated and digested fusion proteins were evaluated for cleavage products by Western blot. The following monoclonal antibodies were used: rat anti-mouse IL-2 antibody (JES6-1A12; ThermoFisher), goat anti-mouse IL-2 polyclonal antibody (AF-419-SP; R&D systems), mouse anti-6×His monoclonal antibody (MA1-21315, ThermoFisher), Anti-mIgG Fc HRP conjugated (ThermoFisher cat #A16084), and Anti-human IL2 antibody (invitrogen, cat #MA5-17097, mouse IgG1). Detection was performed using either a goat anti-rat HRP-conjugated antibody, Donkey Anti-goat HRP-conjugated antibody or Goat Anti-mouse HRP conjugated (Jackson Immuno Research, West Grove, Pa.) and developed using the SuperSignal West Femto Maximum sensitivity detection reagent (ThermoFisher) following the manufacturer's recommendations.

### Example 6: IL-2 Functional Assays

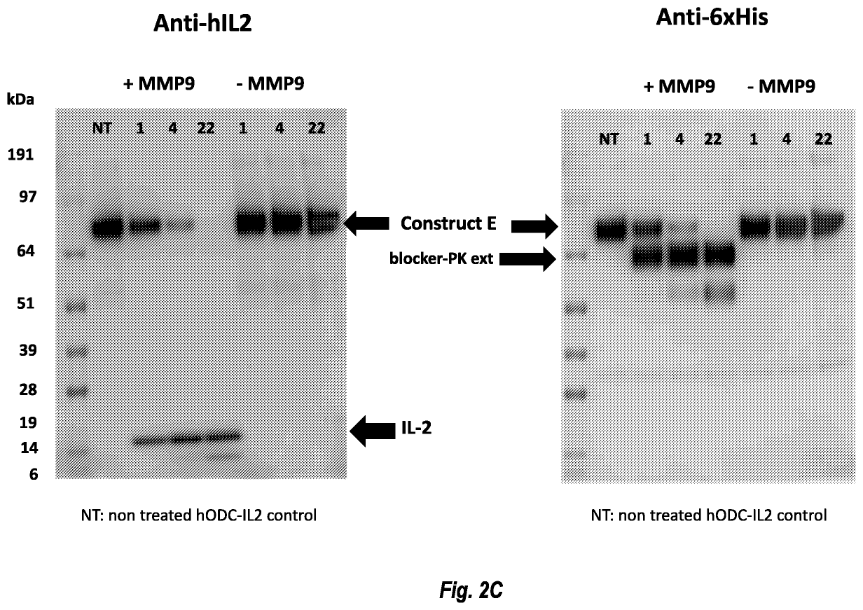

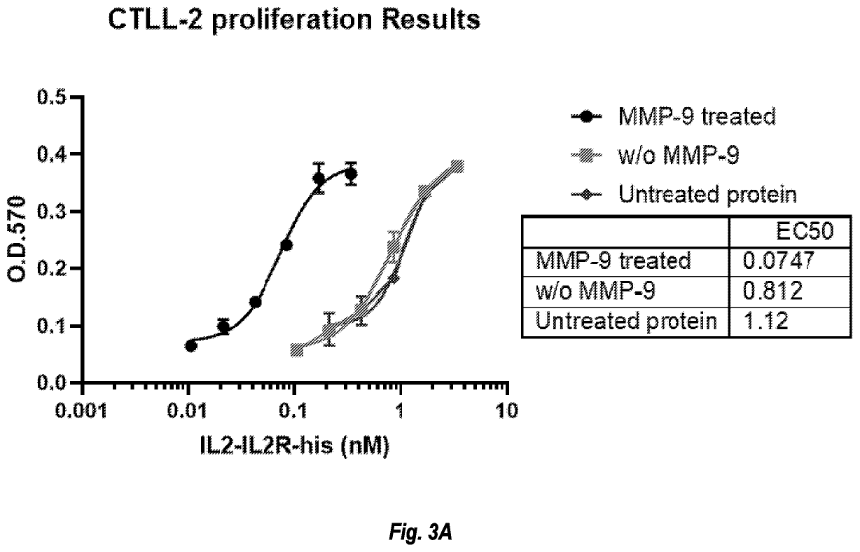

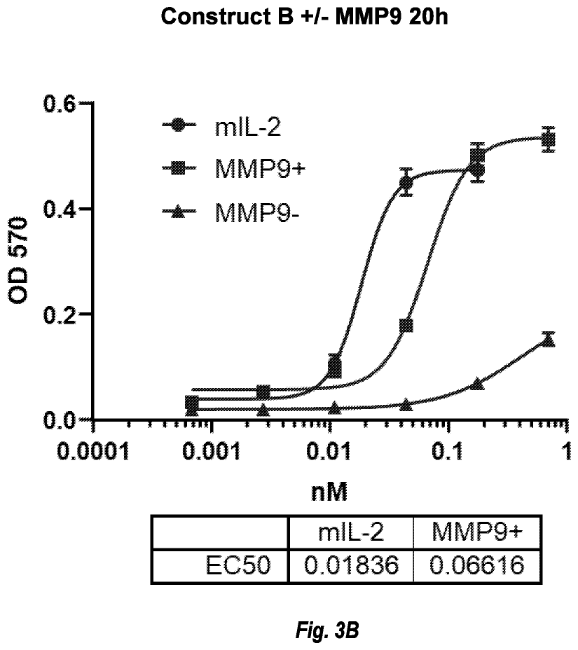

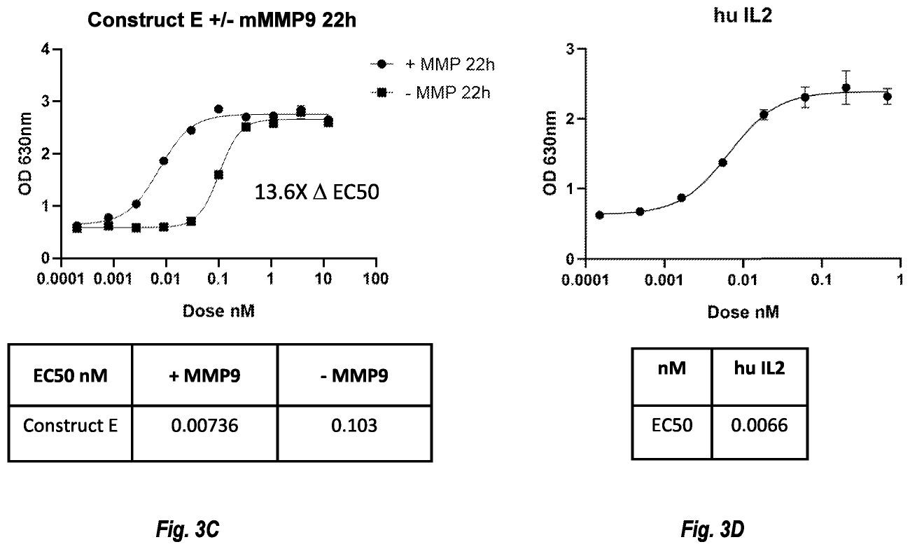

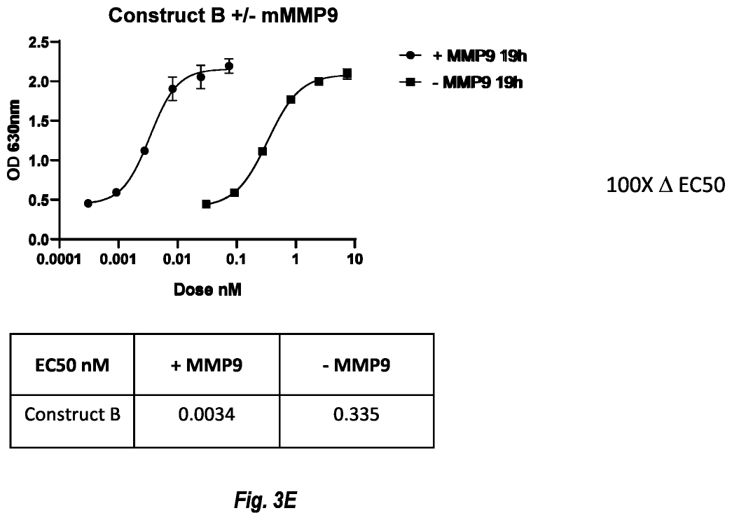

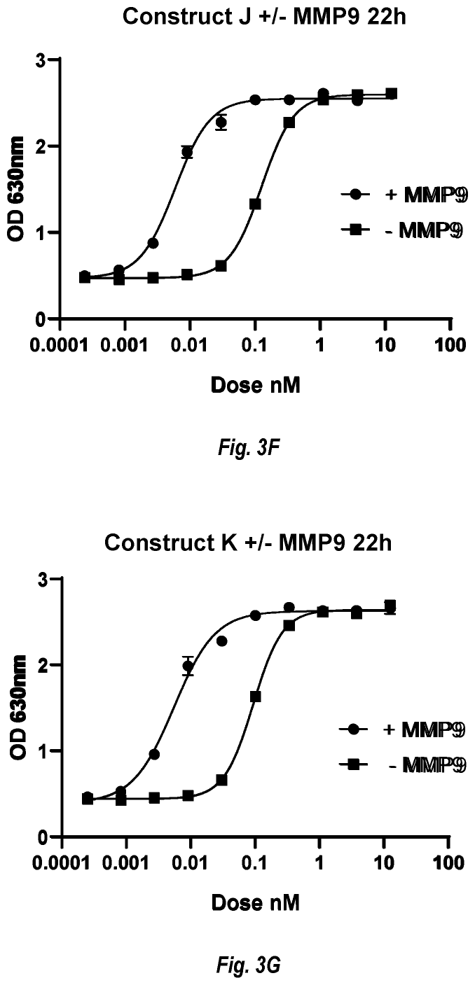

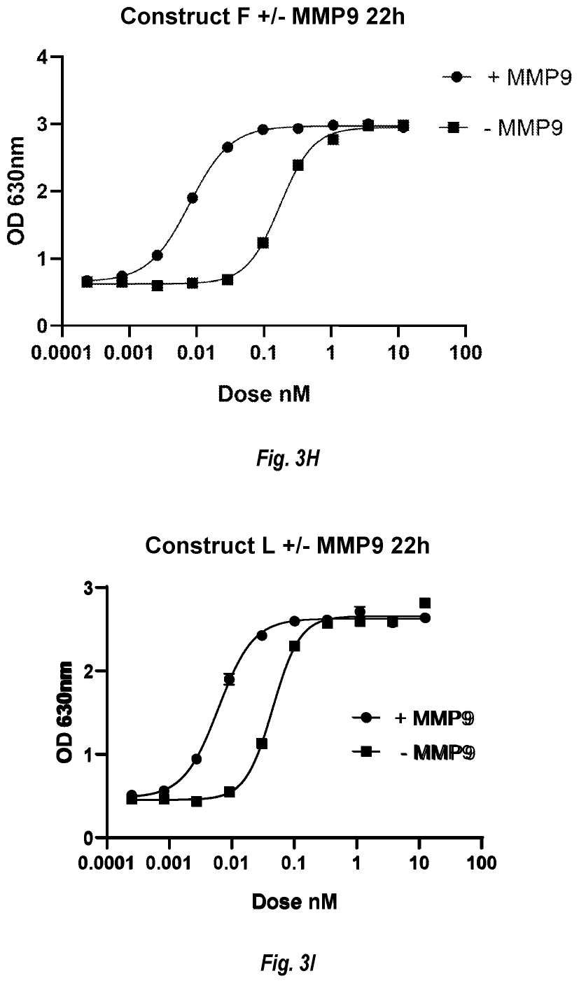

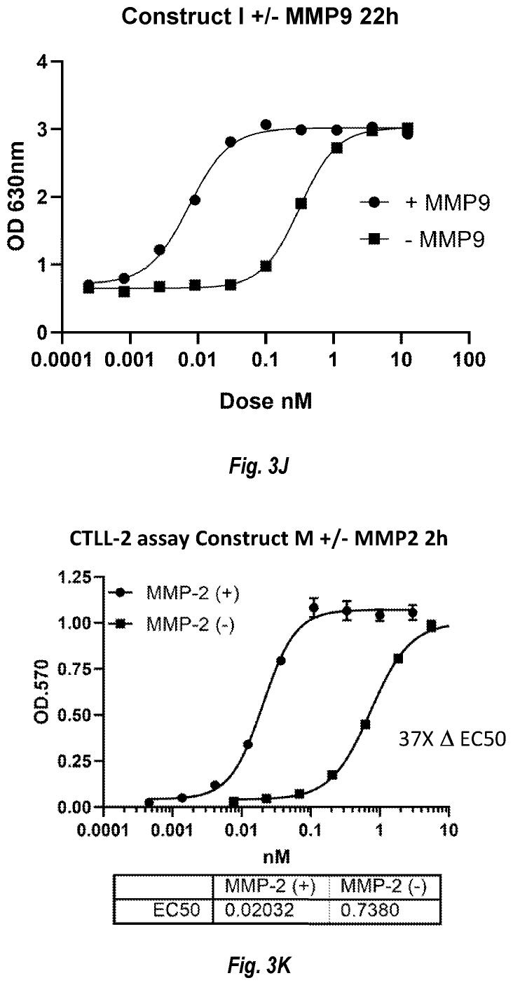

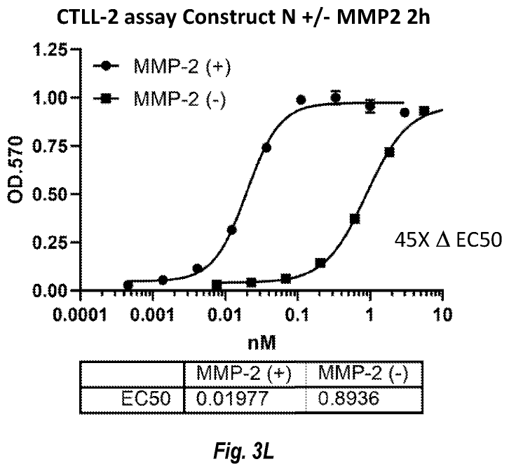

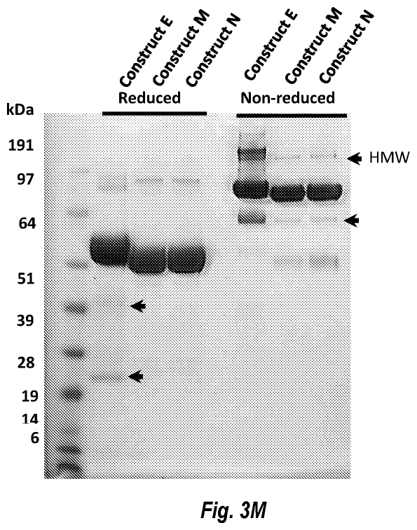

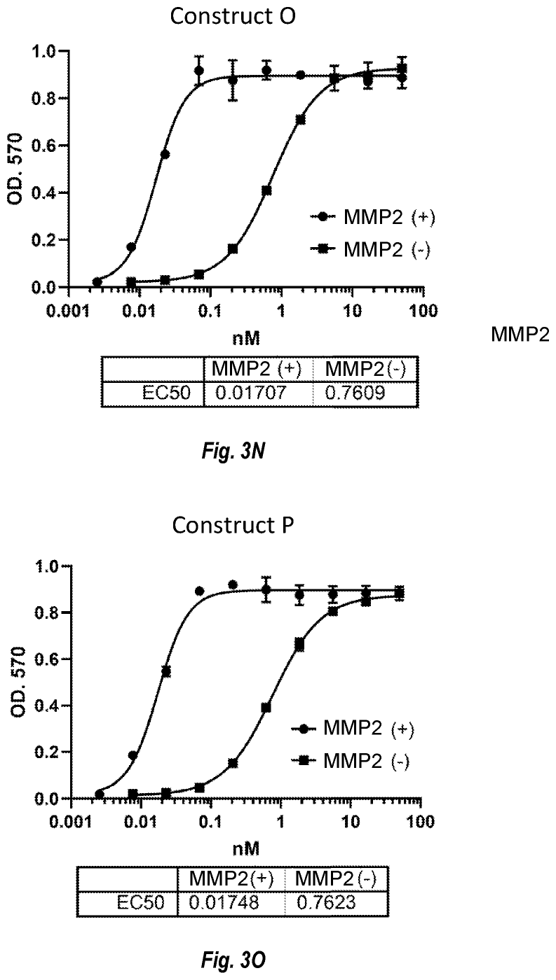

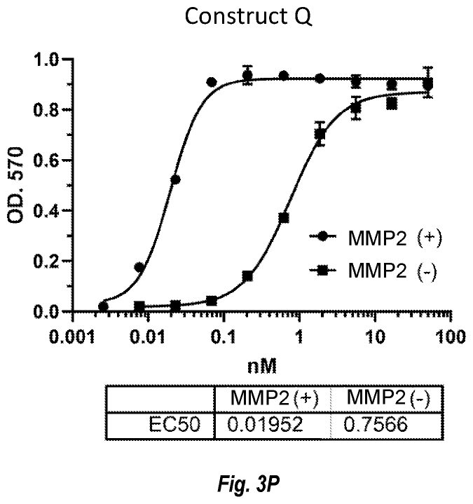

Functional IL-2 was measured using CTLL-2 cells (ATCC) or the reporter cell line HEK Blue IL2 (Invivogen, San Diego). In brief, a titration of digested samples is added to 40 000 CTLL-2 cells per well in 100 ul medium in a 96-well plate and incubated at 37 C in 5% CO2 for 18-22 hr. At the end of this period, 50 ug/well Thiazolyl Blue Tetrazolium Bromide (MTT) (Sigma-Aldrich) was added and the plate was incubated for 5 hr at 37 C in 5% CO2. Cells were lysed with 100 ul/well 10% SDS (Sigma) acidified with HCl, incubated at 37 C for 4 hr, and absorbance was read at 570 nm. Recombinant human or mouse IL-2 (Peprotech and R&D systems respectively) was used as a positive control. FIGS. 3A-B, 3K-L, and 3N-P show examples of untreated and digested fusion proteins evaluated in the CTLL-2 proliferation assay.

HEK-Blue™ IL-2 cells are specifically designed to monitor the activation of the JAK-STAT pathway induced by IL-2. Indeed, stimulation with human or murine IL-2 triggers the JAK/STATS pathway and induces secreted embryonic alkaline phosphatase (SEAP) production. SEAP can be readily monitored when using QUANTI-Blue™, a SEAP detection medium. These cells respond to human and murine IL-2. For the HEK Blue assay, untreated and digested samples were titrated and added to 50 000 HEK Blue cells per well in 200 ul medium in a 96-well plate and incubated at 37 C in 5% CO2 for 20-24 hr. The following day, levels of SEAP were measured by adding 20 uL of cell supernatant to QuantiBlue reagent, followed by 1-3h incubation at 37 C and reading absorbance at 630 nm. FIGS. 3C-J and Table 4 show results obtained from IL2 fusion proteins tested in the HEK Blue™ IL2 assay.

TABLE 4
HEK-Blue ™ IL2 assay results summary

| Construct | +MMP EC50 (nM) | −MMP EC50 (nM) | Fold difference |
|---|---|---|---|
| Construct E | 0.0073 | 0.103 | 14 |
| Construct L | 0.0061 | 0.0453 | 7.3 |
| Construct K | 0.0055 | 0.0933 | 17 |
| Construct J | 0.0078 | 0.1736 | 22 |
| Construct I | 0.0074 | 0.3165 | 43 |

Aggregation, stability, and homogeneity of Construct E, Construct M, and Construct N were compared using Coomassie-stained SDS-PAGE analysis (FIG. 3M). Construct M and Construct N showed decreased aggregation and greater stability and homogeneity, consistent with there being an improvement resulting from deletion of O-glycosylation sites.

### Example 7: In Vitro Serum Stability of Fusion Protein

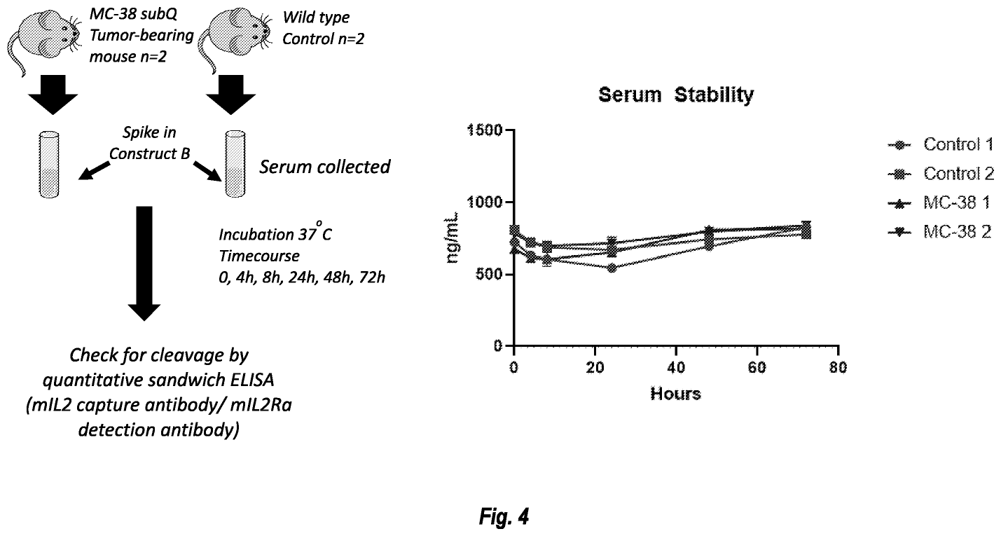

Construct B (SEQ ID NO: 101) was incubated at 37 C for up to 72h with serum collected from 8 weeks old female C57BL/6 naive and MC38 tumor bearing mice respectively (n=2 per serum type, tumor volume >3000 mm3 at time of collection), in order to examine both non-specific cleavage as well as MMP-specific off-target cleavage. Samples were collected at 0h, 4h, 8h, 24 h, 48h and 72h and the intact non-MMP cleaved fusion protein was quantified using an in-house developed sandwich ELISA. Results (see FIG. 4) show that the levels of fusion protein are stable in both serum types, indicating 1) a lack of off-target protein cleavage up to 72 hrs and 2) no active MMPs in circulation.

### Example 8: Pharmacokinetic Evaluation of Fusion Protein in Non-Tumor Bearing Mice

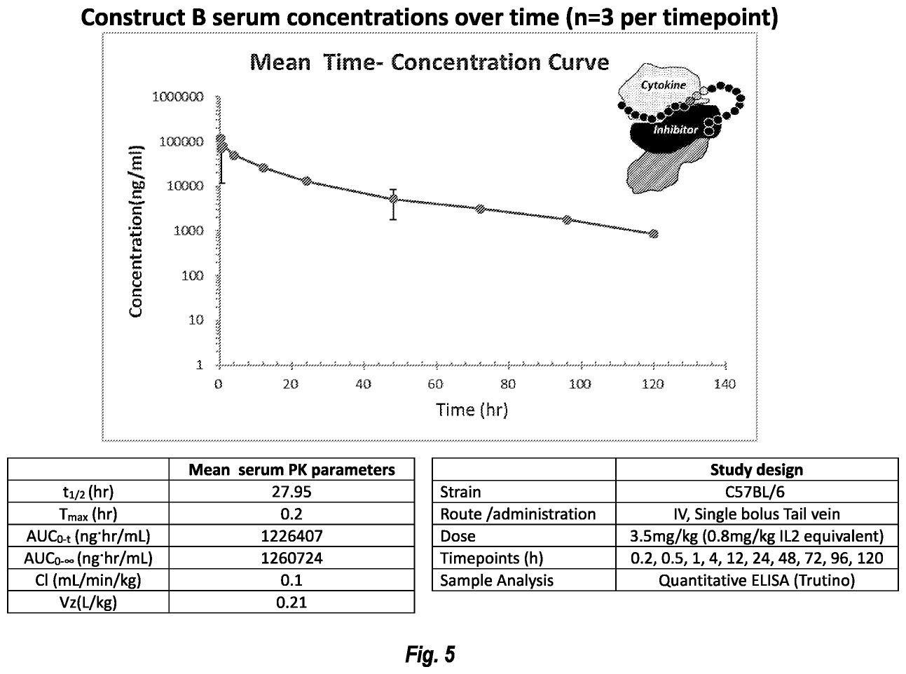

For this study, C57BL/6 8-10 weeks old female mice (Jackson Labs) were assigned to different groups (3 mice per treatment group). Mice received a single dose of fusion protein via IV injection (3.5 mg/kg). 3 mice/group/time point were bled at the following time points: pre-dose (0h), 10 min, 30 min, 1h, 4h, 12h, 24 h, 48h, 72h, 96h and 120h post dose. Blood samples were collected in Eppendorf tubes and processed to serum, then stored at −80 C until testing. Samples were then evaluated by ELISA to quantify intact fusion protein levels. Mean serum concentrations of fusion protein were plotted over time and PK parameters were calculated using WinNonlin 7.0 (non-compartmental model) as shown in FIG. 5.

### Example 9: In Vivo Efficacy of Fusion Proteins in Syngeneic MC38 Colorectal Cancer Model

### Intra-Tumoral Injection of Construct A

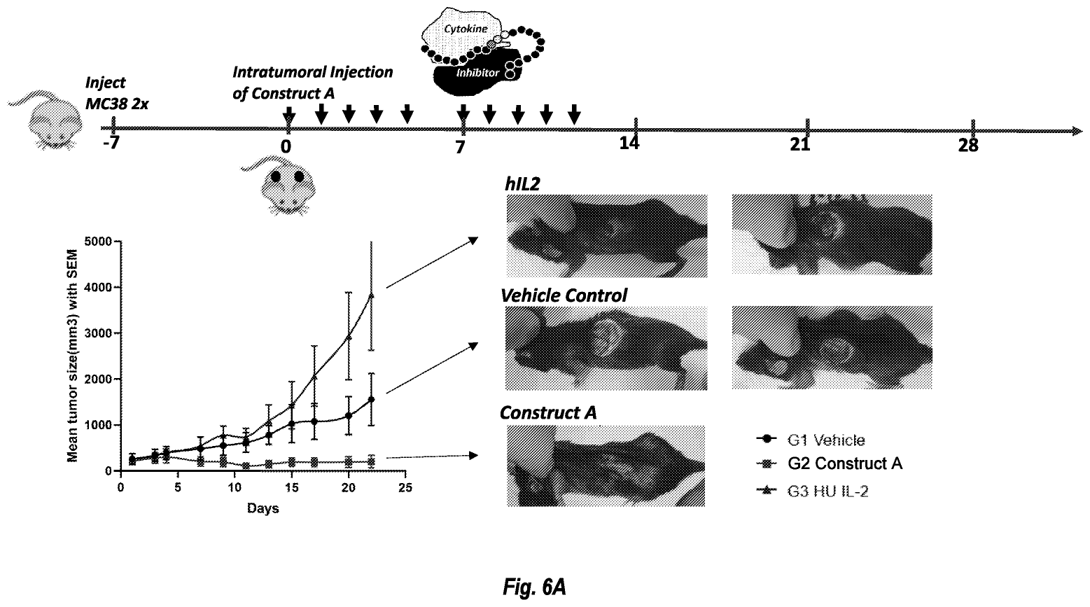

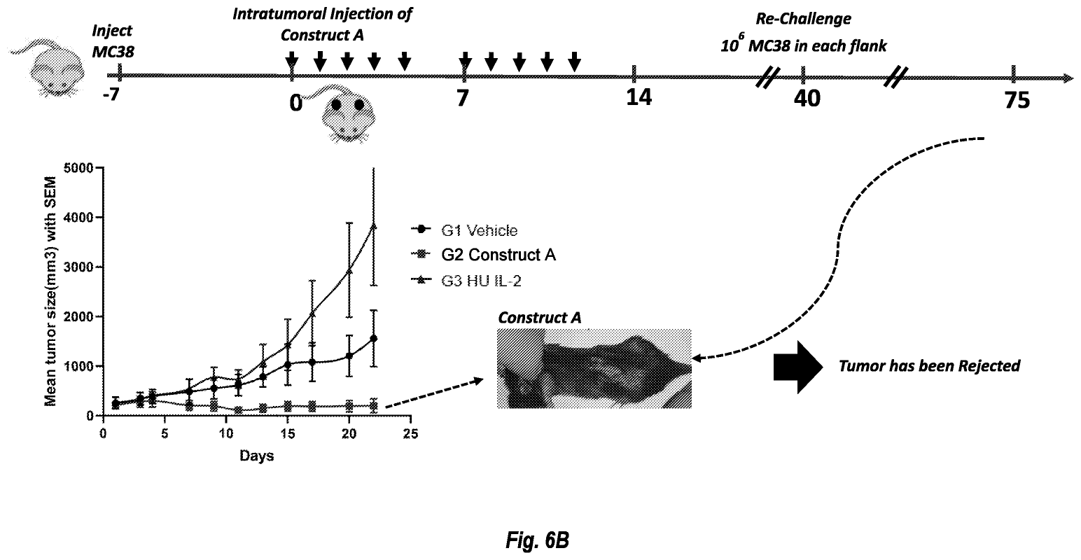

Pilot PK data indicates that Construct A is rapidly cleared from circulation (˜30-fold drop in serum levels within 30 min of IV injection). This is common for small therapeutic proteins whose molecular weight is below the renal glomerular filtration cut-off of ˜60-70 kDa. Hence, this fusion protein was not considered amenable to systemic IV dosing for an in vivo efficacy study. Instead, a direct intra-tumoral delivery design was used with 3 arms: vehicle, recombinant human IL-2 (r hIL2) and Construct A (n=3 mice/arm). IL-2 has previously demonstrated anti-tumor activity in a variety of syngeneic models by direct tumor injection, and based on this data, r hIL2 was dosed at 5 ug/day (equivalent to 50 000 U/day). Construct A was dosed at 70 ug/day, which represents a 5 molar excess compared to recombinant IL-2 to compensate for the EC50 difference observed in the CTLL-2 assay. All agents and vehicle were injected daily into subcutaneous MC38 tumor mass (˜200 mm³ in size upon initiation of dosing) growing on the flank of C57BL/6 mice for 12 days with 2-day holiday after first 5 injections (total of 10 injections). Tumors and body weights were measured twice a week for the duration of the study. Tumor volumes were calculated using the following equation: (longest diameter*shortest diameter²)/2. As shown in FIG. 6A, significant anti-tumor activity was observed for Construct A. Indeed, a complete elimination of tumor was observed in the Construct A treatment group while no tumor regression was observed in either vehicle or r hIL2 treatment groups. When ‘cured’ Construct A-treated mice were re-inoculated with MC38 tumor cells (10⁶ cells on opposite flank) on Day 40, no tumor mass was established a month after re-challenge, suggesting the existence of a ‘memory’ immune response in these mice (FIG. 6B).

### Systemic IV Injection of Construct B

The objective of this study was to evaluate efficacy of Construct B in the MC38-bearing female C57BL/6 mice. For this study, C57BL/6 6-8 weeks old female mice (Jackson Labs) were subcutaneously inoculated with MC38 cells (10⁶ cells/animal), and when the average tumor volume reached about 80 mm³, animals were randomized into 2 groups based on tumor volumes (8 mice per treatment group). Animals were dosed according to the following study design:

TABLE 5

| | | | | Dosing | Dose | |
|---|---|---|---|---|---|---|
| | | | Dose | Frequency & | Level | Dose |
| Group | Treatment | N | Route | Duration | (mpk) | Volume (ul) |
| 1 | Vehicle | 8 | IV | Q3D for 21D | N/A | 100 |
| | Control | | | | | |
| 2 | Construct B | 8 | IV | Q3D for 21D | 10 | 100 |

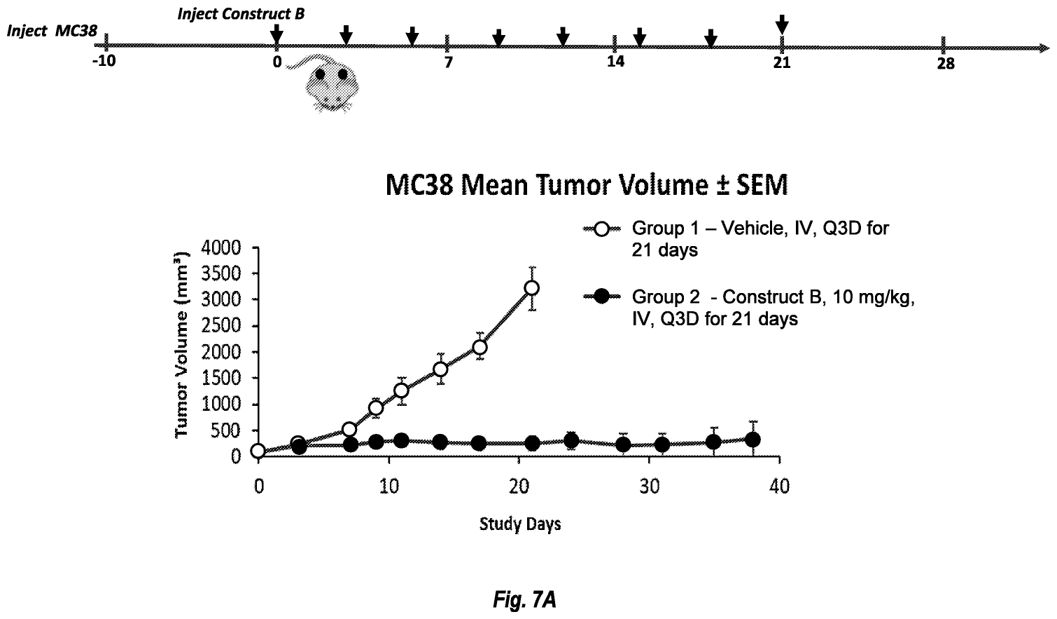

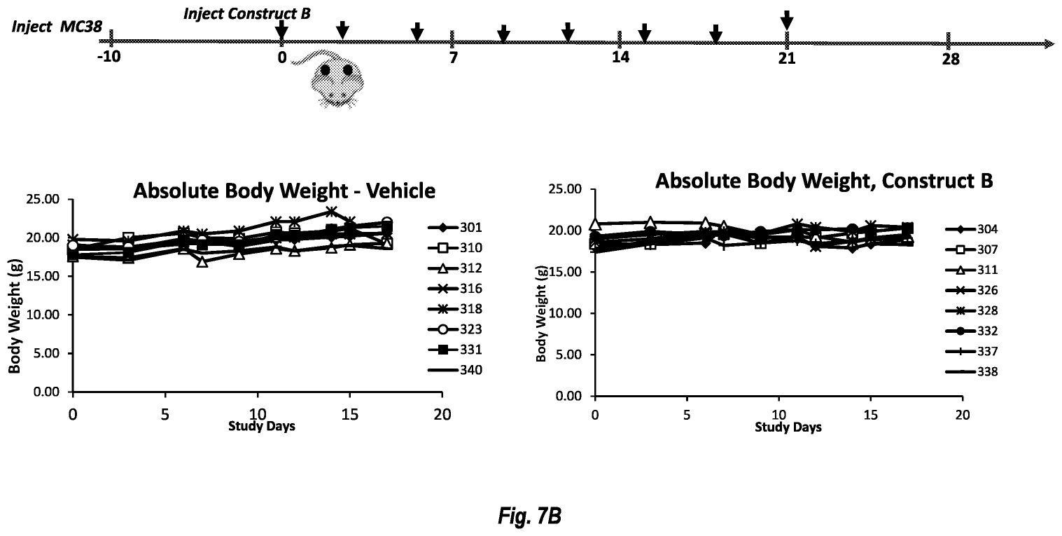

Mice were dosed over a 21 day period then further observed for an additional week. Tumors and body weights were measured twice a week for the duration of the study. Tumor volumes were calculated using the following equation: (longest diameter*shortest diameter²)/2. FIG. 7A-B show the mean tumor volume over time for both groups (FIG. 7A) and individual body weights of vehicle and treated (FIG. 7B) animals.

The results showed excellent efficacy for the treatment group, with 92% inhibition of tumor growth at Day 21, while no adverse effect was observed. Out of 8 cases, 3 complete tumor regressions (‘cures’) occurred in the colorectal cancer syngeneic setting.

### Example 10: Evaluation of Immune Cell Populations by Immunohistochemistry (IHC) in MC38 Colorectal Cancer Samples

Immune targets in tumor samples were evaluated by IHC, specifically, CD4+Foxp3 double immunofluorescence staining and CD8, CD25, CD3, CD4 and CD335 single IHC staining. Prior to performing IHC, H&E staining was run for all control and Construct B treated tumors to check the tissue quality.

7 tumor samples were selected from the systemic in vivo efficacy study and formalin-fixed paraffin embedded (FFPE) blocks were prepared following a standard embedding process.

TABLE 6
Model type: MC38

| | | Number of |
|---|---|---|
| Group | Treatment | FFPE blocks |
| 1 | Vehicle, IV, Q3D for 21 days | 4 |
| 2 | Construct B, 10 mg/kg, IV, Q3D for 21 days | 3 |

The following antibodies and other materials were used:

TABLE 7
Antibodies and Other Materials

| Antibody/ | | | | |
|---|---|---|---|---|
| other | | | | |
| material | Company | Cat# | Type | Reactivity |
| CD4 | Cell | 25229 | Rabbit IgG | Mouse |
| | Signaling | | mAb | |
| FoxP3 | Cell | 12653 | Rabbit IgG | Mouse |
| | Signaling | | mAb | |
| CD8 | Cell | 98941 | Rabbit IgG | Mouse |
| | Signaling | | mAb | |
| CD25 | abeam | ab227834 | Rabbit IgG | Mouse |
| | | | mAb | |
| CD3 | Cell | 99940 | Rabbit IgG | Mouse |
| | Signaling | | mAb | |
| CD335 | R&D | AF2225-SP | Goat IgG pAb | Mouse |
| | Systems | | | |
| Bond | Leica | DS9800 | Anti-rabbit Poly-HRP-IgG | |
| Polymer | | | (<25 μg/mL) containing 10% | |
| Refine | | | (v/v) animal serum in tris- | |
| Detection | | | buffered saline/0.09% | |
| | | | ProClin ™ 950 (ready-to-use) | |
| ImmPRESS | Vector | MP-7405 | Anti-goat Poly-HRP-IgG | |
| HRP Anti- | | | (<25 μg/mL) containing 10% | |
| Goat Ig | | | (v/v) animal serum in tris- | |
| | | | buffered saline (ready-to-use) | |
| | | | and House serum (2.5%) | |
| Rabbit | Cell | 3900 | Isotype control | |
| (DAIE) | Signaling | | | |
| mAb IgG | | | | |
| TRITC | PerkinElmer | NEL742001KT | Fluorescent | |
| TSA(Red) | | | double staining | |
| FITC | PerkinElmer | NEL741001KT | Fluorescent | |
| TSA(Green) | | | double staining | |

FFPE blocks were sectioned with a manual rotary microtome (4 μm thickness/section) and optimized IHC assay protocols for all the antibodies were used. All stained sections were scanned with NanoZoomer-S60 Image system with 40× magnification. High resolution picture for whole section was generated and further analyzed.

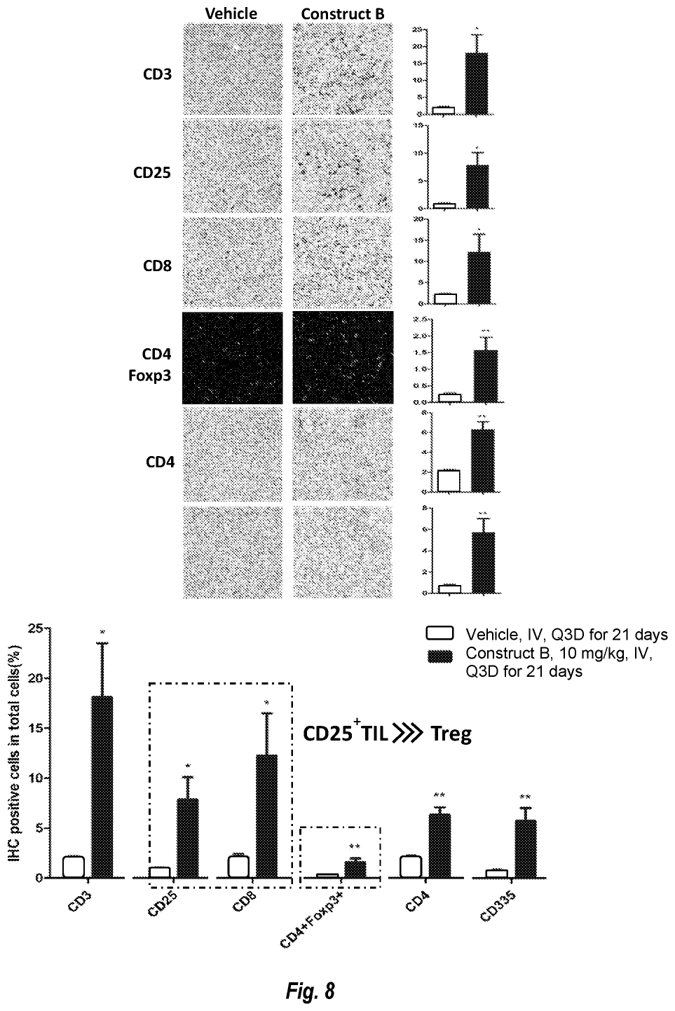

Scoring Method: All the images were analyzed with HALO′ Image Analysis platform. The whole slide image was analyzed and necrosis area was excluded. The total cells and IHC positive cells were counted. IHC score is presented as the ratio of the positive cell counts against the total cell numbers within whole section and shown in FIG. 8. Results show that there is a significant increase in tumor infiltrating immune cells post Construct B treatment.

### Example 11: In Vivo MMP Activity Evaluation in Diverse Syngeneic Tumor Models

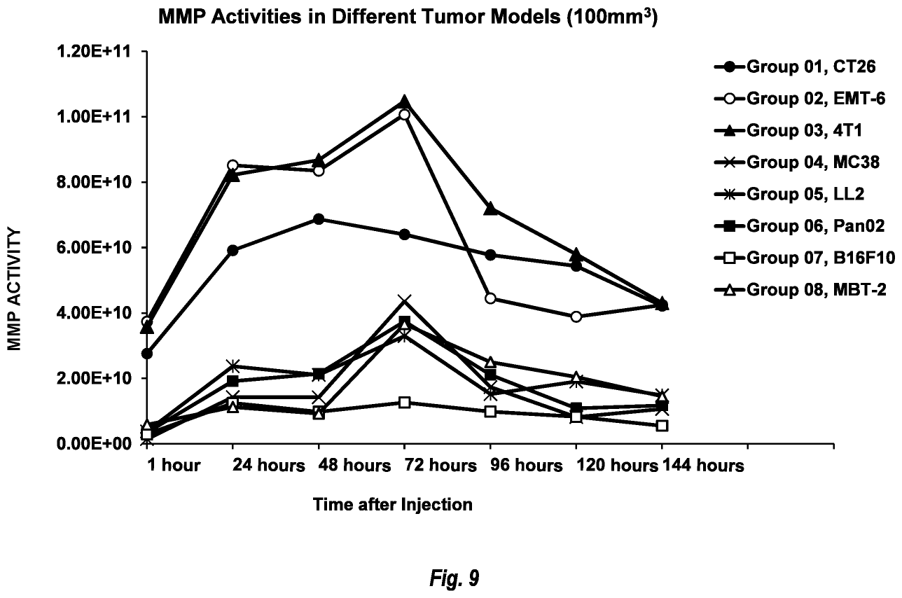

The degree of MMP activity was assessed in vivo utilizing an MMP-activatable fluorescent probe, MMPSense 680™. This probe is optically silent in its intact state and becomes highly fluorescent following MMP-mediated cleavage and is designed to be used as a real-time in vivo imaging tool (Perkin Elmer). Following a single dose IV injection of the probe to tumor-bearing mice, fluorescent images were captured over 6 days and the fluorescence intensity in tumor area, which is directly proportional to MMP activity present, was quantified (FIG. 9). All models showed MMP activity with some variation between different tumor types.

### Example 12: In Vivo Efficacy of Construct B in Diverse Syngeneic Tumor Models

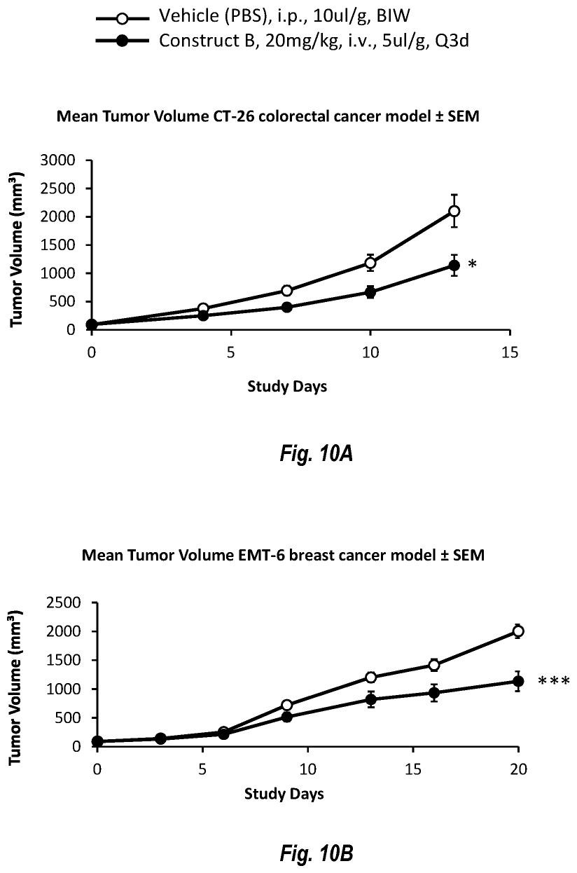

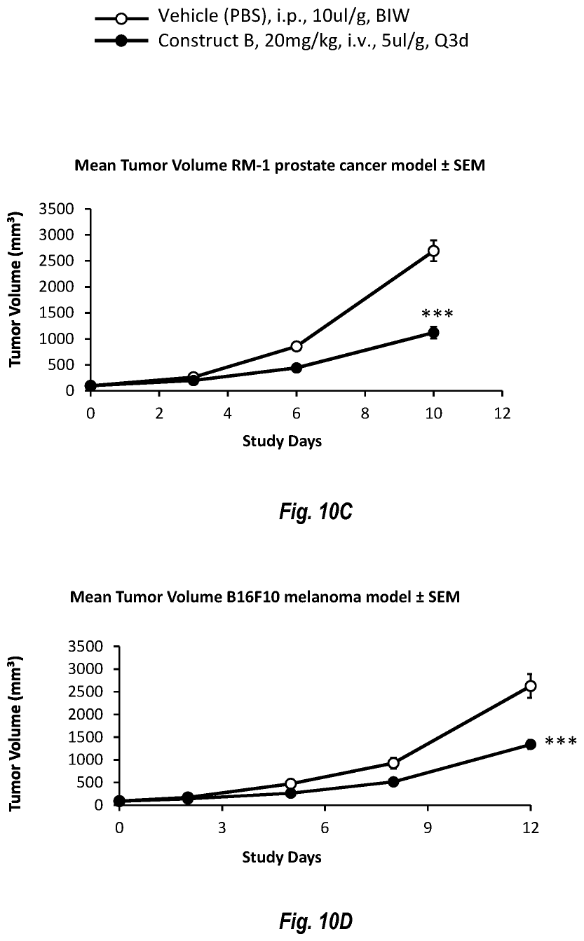

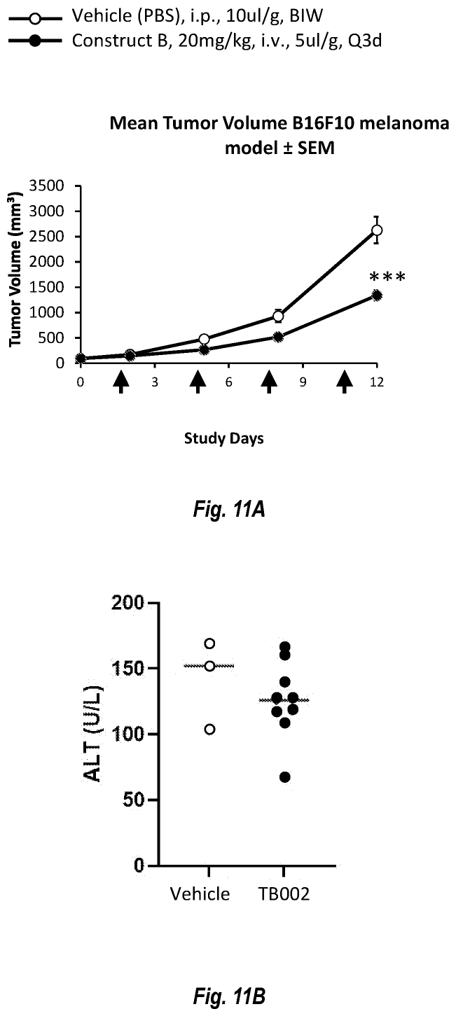

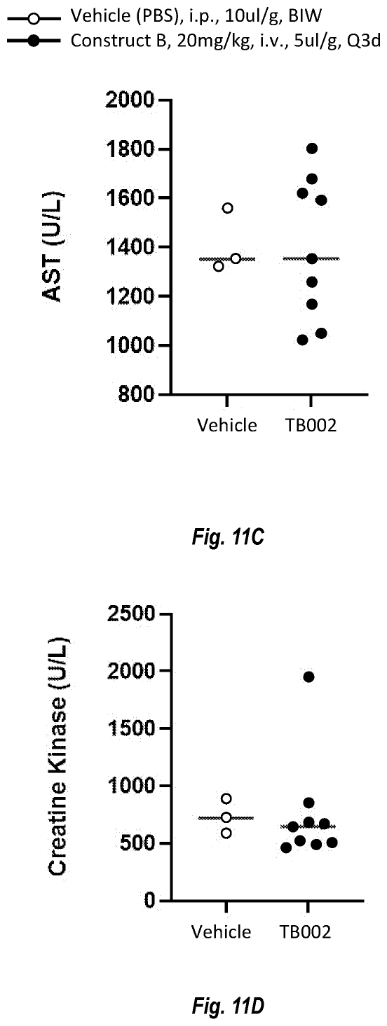

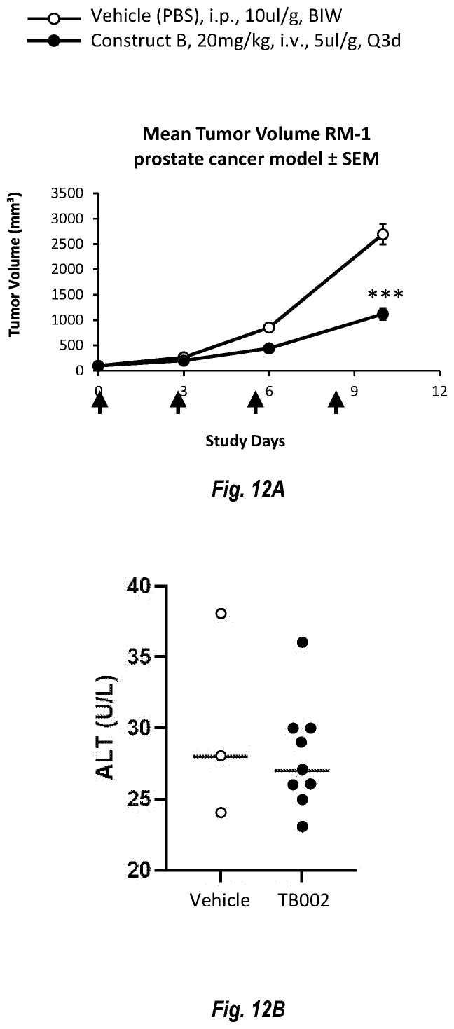

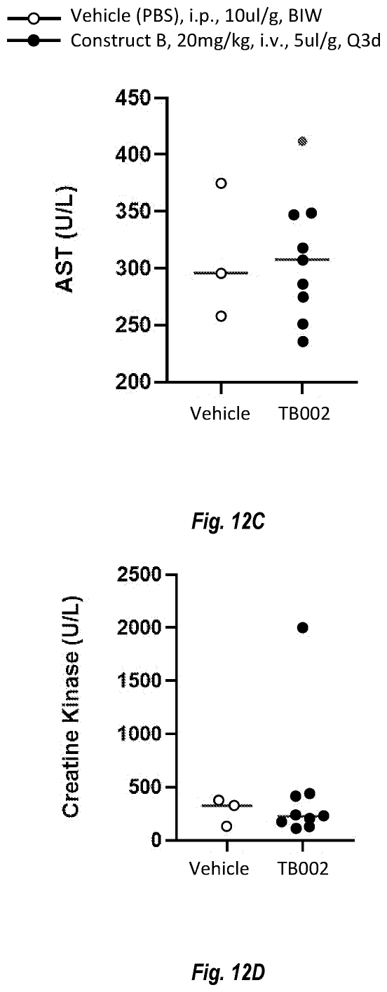

C57BL/6 or BALB/c mice were subcutaneously inoculated with malignant cells and when the average tumor volume reached on average 90 mm³, animals were randomized into 2 groups based on tumor volumes (n=10 mice per treatment group). Mice were dosed intravenously every 3 days (Q3D) at 20 mg/kg. Tumors, body weights and clinical observations were measured/collected twice a week for the duration of the study. Tumor volume is shown in FIGS. 10A-D, 11A, 12A, and 13B-C. Robust anti-tumor activity was observed in several models. Notably, 49% tumor growth inhibition (TGI) was observed at Day 12 in the B16F10 melanoma model and 58% tumor TGI was observed at Day 10 in the aggressive Ras/Myc transformed RM-1 prostate cancer model (FIG. 10C and Table 8). Notably, no signs of toxicity, including body weight loss and elevated levels of liver and/or kidney enzymes, were noted and clinical observations were normal in these models. Liver and kidney enzyme results corresponding to FIGS. 11A and 12A are shown in FIGS. 11B-D and 12B-D, respectively.

TABLE 8
Tumor growth inhibition (TGI) results

| | Mouse | | Max TGI % | p value* | |
|---|---|---|---|---|---|
| Cancer type | Strain | Model | (day observed) | (T test) | MMP score |
| Breast | BALB/c | EMT06 | 43 (20) | 0.0006 | High |
| Melanoma | C57BL/6 | B16F10 | 49 (12) | 0.0004 | Low |
| Colorectal | BALB/c | CT-26 | 46 (13) | 0.0114 | Med/high |
| Colorectal | C57BL/6 | MC-38 | 92 (21) | <0.0001 | Med |
| Prostate | C57BL/6 | RM_1 | 58 (10) | <0.0001 | Not |
| | | | | | determined |

*p values were determined using unpaired t test (graphpad prism) between vehicle and Construct B groups on day of max TGI.

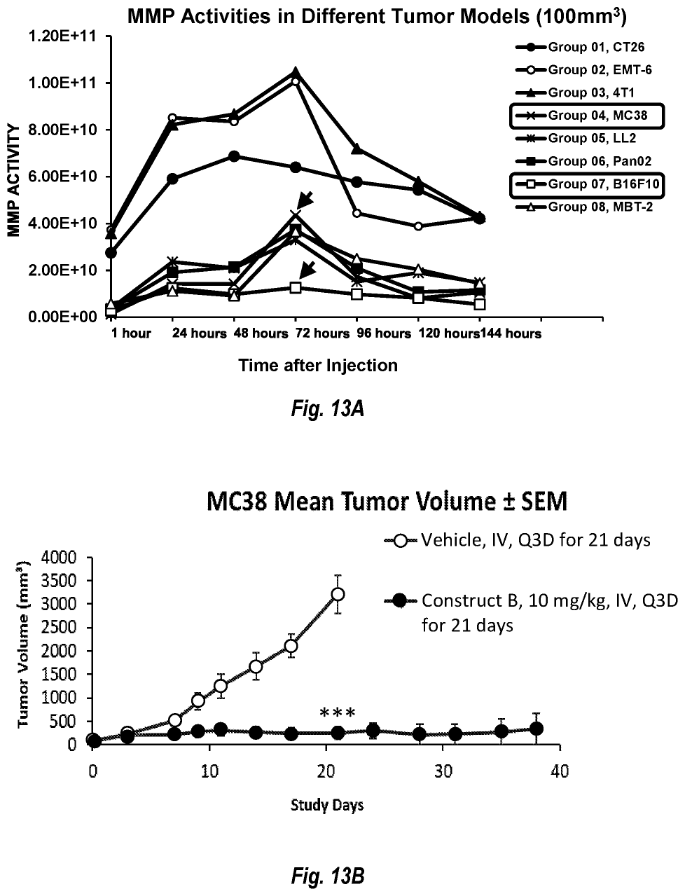

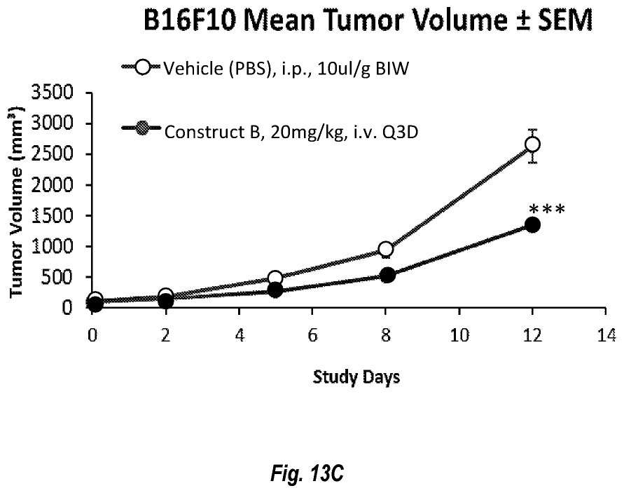

The difference in efficacy between MC-38 and B16F10 models may in part be due to the lower MMP activity measured in B16F10 tumors (FIG. 13A), resulting in less functional IL-2 being released in the TME relative to the MC38 setting.

We claim:

1. A protease-activated pro-cytokine comprising:
a cytokine polypeptide sequence;
a inhibitory polypeptide sequence capable of blocking an activity of the cytokine polypeptide sequence; and
a linker between the cytokine polypeptide sequence and the inhibitory polypeptide sequence, the linker comprising a protease-cleavable polypeptide sequence;
wherein:
i) the protease-cleavable polypeptide sequence is a protease-cleavable polypeptide sequence comprising any one of SEQ ID NOs: 80-94 or 201-242, or a variant having one or two mismatches relative to the sequence of any one of SEQ ID NOs: 80-90 or 201-242.

2. The protease-activated pro-cytokine of the immediately preceding claim, further comprising a pharmacokinetic modulator.

3. The protease-activated pro-cytokine of the immediately preceding claim, wherein the pharmacokinetic modulator comprises an immunoglobulin constant domain.

4. The protease-activated pro-cytokine of claim 2, wherein the pharmacokinetic modulator comprises an immunoglobulin Fc region, optionally wherein the Fc region is a knob-into-hole heterodimeric Fc region.

5. The protease-activated pro-cytokine of the immediately preceding claim, wherein the immunoglobulin Fc region is a human immunoglobulin Fc region.

6. The protease-activated pro-cytokine of any one of claims 4-5, wherein the immunoglobulin Fc region is an IgG Fc region.

7. The protease-activated pro-cytokine of the immediately preceding claim, wherein the IgG Fc region is an IgG1, IgG2, IgG3, or IgG4 Fc region.

8. The protease-activated pro-cytokine of claim 2, wherein the pharmacokinetic modulator comprises an albumin.

9. The protease-activated pro-cytokine of the immediately preceding claim, wherein the albumin is a serum albumin.

10. The protease-activated pro-cytokine of any one of claims 8-9, wherein the albumin is a human albumin.

11. The protease-activated pro-cytokine of claim 2, wherein the pharmacokinetic modulator comprises PEG.

12. The protease-activated pro-cytokine of claim 2, wherein the pharmacokinetic modulator comprises XTEN.

13. The protease-activated pro-cytokine of claim 2, wherein the pharmacokinetic modulator comprises CTP.

14. The protease-activated pro-cytokine of any one of claims 2-13, wherein the protease-cleavable polypeptide sequence is between the cytokine polypeptide sequence and the pharmacokinetic modulator.

15. The protease-activated pro-cytokine of any one of claims 2-13, wherein the pharmacokinetic modulator is between the cytokine polypeptide sequence and the protease-cleavable polypeptide sequence.

16. The protease-activated pro-cytokine of any one of the preceding claims, comprising a plurality of protease-cleavable polypeptide sequences.

17. The protease-activated pro-cytokine of the immediately preceding claim, wherein the cytokine polypeptide sequence is flanked by protease cleavable polypeptide sequences.

18. The protease-activated pro-cytokine of the immediately preceding claim, having the structure PM-CL-CY-CL-IN (from N- to C-terminus or from C- to N-terminus), where PM is the pharmacokinetic modulator, each CL independently is a protease-cleavable polypeptide sequence, CY is the cytokine polypeptide sequence, and IN is the inhibitory polypeptide sequence.

19. The protease-activated pro-cytokine of any one of the preceding claims, wherein the cytokine polypeptide sequence comprises a modification to prevent disulfide bond formation, and optionally otherwise comprises wild-type sequence.

20. The protease-activated pro-cytokine of any one of the preceding claims, wherein the cytokine polypeptide sequence has at least 80, 85, 90, 95, 97, 98, or 99 percent identity to the sequence of a wild-type cytokine polypeptide sequence or to a cytokine polypeptide sequence in Table 1.

21. The protease-activated pro-cytokine of the immediately preceding claim, wherein the cytokine polypeptide sequence is a wild-type cytokine polypeptide sequence.

22. The protease-activated pro-cytokine of any one of the preceding claims, wherein the cytokine is a monomeric cytokine or a dimeric cytokine, wherein the monomers are associated noncovalently or covalently directly or indirectly via a linker.

23. The protease-activated pro-cytokine of any one of the preceding claims, wherein the inhibitory polypeptide sequence comprises a cytokine-binding domain.

24. The protease-activated pro-cytokine of the immediately preceding claim, wherein the cytokine-binding domain is a cytokine-binding domain of a cytokine receptor or a cytokine-binding domain of a fibronectin.

25. The protease-activated pro-cytokine of the immediately preceding claim, wherein the cytokine-binding domain comprises the sequence of any one of SEQ ID NOs: 10-29 or 40-51.

26. The protease-activated pro-cytokine of claim 24, wherein the cytokine-binding domain is an immunoglobulin cytokine-binding domain.

27. The protease-activated pro-cytokine of the immediately preceding claim, wherein the immunoglobulin cytokine-binding domain comprises a light chain variable domain and a heavy chain variable domain that bind the cytokine.

28. The protease-activated pro-cytokine of any one of claims 26-27, wherein the immunoglobulin cytokine-binding domain is an scFv or Fab.

29. The protease-activated pro-cytokine of any one of the preceding claims, wherein the protease-cleavable polypeptide sequence is cleavable by at least one of a metalloprotease, a serine protease, a cysteine protease, an aspartate protease, a threonine protease, a glutamate protease, a gelatinase, an asparagine peptide lyase, a cathepsin, a kallikrein, a plasmin, a collagenase, a hK1, a hK10, a hK15, a stromelysin, a Factor Xa, a chymotrypsin-like protease, a trypsin-like protease, a elastase-like protease, a subtilisin-like protease, an actinidain, a bromelain, a calpain, a caspase, a Mir 1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, an ADAM 10, an ADAM17, an ADAM 12, an urokinase plasminogen activator (uPA), an enterokinase, a prostate-specific target (PSA, hK3), an interleukin-1b converting enzyme, a thrombin, a FAP (FAP-a), a dipeptidyl peptidase, or dipeptidyl peptidase IV (DPPIV/CD26), a type II transmembrane serine protease (TTSP), a neutrophil elastase, a proteinase 3, a mast cell chymase, a mast cell tryptase, or a dipeptidyl peptidase.

30. The protease-activated pro-cytokine of any one of the preceding claims, wherein the protease-cleavable polypeptide sequence comprises the sequence of any one of SEQ ID NOs: 201-242, or a variant having one or two mismatches relative to the sequence of any one of SEQ ID NOs: 201-242.

31. The protease-activated pro-cytokine of any one of the preceding claims, wherein the protease-cleavable polypeptide sequence is cleavable by a matrix metalloprotease.

32. The protease-activated pro-cytokine of any one of the preceding claims, wherein the protease-cleavable polypeptide sequence is cleavable by MMP-1.

33. The protease-activated pro-cytokine of any one of the preceding claims, wherein the protease-cleavable polypeptide sequence is cleavable by MMP-2.

34. The protease-activated pro-cytokine of any one of the preceding claims, wherein the protease-cleavable polypeptide sequence is cleavable by MMP-3.

35. The protease-activated pro-cytokine of any one of the preceding claims, wherein the protease-cleavable polypeptide sequence is cleavable by MMP-7.

36. The protease-activated pro-cytokine of any one of the preceding claims, wherein the protease-cleavable polypeptide sequence is cleavable by MMP-8.

37. The protease-activated pro-cytokine of any one of the preceding claims, wherein the protease-cleavable polypeptide sequence is cleavable by MMP-9.

38. The protease-activated pro-cytokine of any one of the preceding claims, wherein the protease-cleavable polypeptide sequence is cleavable by MMP-12.

39. The protease-activated pro-cytokine of any one of the preceding claims, wherein the protease-cleavable polypeptide sequence is cleavable by MMP-13.

40. The protease-activated pro-cytokine of any one of the preceding claims, wherein the protease-cleavable polypeptide sequence is cleavable by MMP-14.

41. The protease-activated pro-cytokine of any one of the preceding claims, wherein the protease-cleavable polypeptide sequence is cleavable by more than one MMP.

42. The protease-activated pro-cytokine of any one of the preceding claims, wherein the protease-cleavable polypeptide sequence is cleavable by two, three, four, five, six, or seven of MMP-2, MMP-7, MMP-8, MMP-9, MMP-12, MMP-13, and MMP-14.

43. The protease-activated pro-cytokine of any one of the preceding claims, wherein the protease-cleavable polypeptide sequence comprises the sequence of any one of SEQ ID NOs: 80-94 or a variant sequence having one or two mismatches relative to the sequence of any one of SEQ ID NOs: 80-90.

44. The protease-activated pro-cytokine of the immediately preceding claim, wherein the protease-cleavable polypeptide sequence comprises the sequence of SEQ ID NO: 80 or a variant sequence having one or two mismatches relative thereto.

45. The protease-activated pro-cytokine of any one of claims 1-43, wherein the protease-cleavable polypeptide sequence comprises the sequence of SEQ ID NO: 81 or a variant sequence having one or two mismatches relative thereto.

46. The protease-activated pro-cytokine of any one of claims 1-43, wherein the protease-cleavable polypeptide sequence comprises the sequence of SEQ ID NO: 82 or a variant sequence having one or two mismatches relative thereto.

47. The protease-activated pro-cytokine of any one of claims 1-43, wherein the protease-cleavable polypeptide sequence comprises the sequence of SEQ ID NO: 83 or a variant sequence having one or two mismatches relative thereto.

48. The protease-activated pro-cytokine of any one of claims 1-43, wherein the protease-cleavable polypeptide sequence comprises the sequence of SEQ ID NO: 84 or a variant sequence having one or two mismatches relative thereto.

49. The protease-activated pro-cytokine of any one of claims 1-43, wherein the protease-cleavable polypeptide sequence comprises the sequence of SEQ ID NO: 85 or a variant sequence having one or two mismatches relative thereto.

50. The protease-activated pro-cytokine of any one of claims 1-43, wherein the protease-cleavable polypeptide sequence comprises the sequence of SEQ ID NO: 86 or a variant sequence having one or two mismatches relative thereto.

51. The protease-activated pro-cytokine of any one of claims 1-43, wherein the protease-cleavable polypeptide sequence comprises the sequence of SEQ ID NO: 87 or a variant sequence having one or two mismatches relative thereto.

52. The protease-activated pro-cytokine of any one of claims 1-43, wherein the protease-cleavable polypeptide sequence comprises the sequence of SEQ ID NO: 88 or a variant sequence having one or two mismatches relative thereto.

53. The protease-activated pro-cytokine of any one of claims 1-43, wherein the protease-cleavable polypeptide sequence comprises the sequence of SEQ ID NO: 89 or a variant sequence having one or two mismatches relative thereto.

54. The protease-activated pro-cytokine of any one of claims 1-43, wherein the protease-cleavable polypeptide sequence comprises the sequence of SEQ ID NO: 90 or a variant sequence having one or two mismatches relative thereto.

55. The protease-activated pro-cytokine of any one of claims 1-43, wherein the protease-cleavable polypeptide sequence comprises the sequence of SEQ ID NO: 80-89 or 90.

56. The protease-activated pro-cytokine of any one of claims 1-43, wherein the protease-cleavable polypeptide sequence comprises the sequence of SEQ ID NO: 91.

57. The protease-activated pro-cytokine of any one of claims 1-43, wherein the protease-cleavable polypeptide sequence comprises the sequence of SEQ ID NO: 92.

58. The protease-activated pro-cytokine of any one of claims 1-43, wherein the protease-cleavable polypeptide sequence comprises the sequence of SEQ ID NO: 93.

59. The protease-activated pro-cytokine of any one of claims 1-43, wherein the protease-cleavable polypeptide sequence comprises the sequence of SEQ ID NO: 94.

60. The protease-activated pro-cytokine of any one of the preceding claims, wherein the cytokine polypeptide sequence is an IL-2 polypeptide sequence.

61. The protease-activated pro-cytokine of the immediately preceding claim, wherein the IL-2 polypeptide sequence has at least 80, 85, 90, 95, 97, 98, or 99 percent identity to the sequence of any one of SEQ ID NOs: 1-4.

62. The protease-activated pro-cytokine of the immediately preceding claim, wherein the IL-2 polypeptide sequence comprises the sequence of any one of SEQ ID NOs: 1-4.

63. The protease-activated pro-cytokine of any one of claims 60-62, wherein the IL-2 polypeptide sequence is a human IL-2 polypeptide sequence.

64. The protease-activated pro-cytokine of the immediately preceding claim, wherein the IL-2 polypeptide sequence comprises the sequence of SEQ ID NO: 1.

65. The protease-activated pro-cytokine of any one of claim 62, wherein the IL-2 polypeptide sequence comprises the sequence of SEQ ID NO: 2.

66. The protease-activated pro-cytokine of any one of claims 60-65, wherein the inhibitory polypeptide sequence comprises an IL-2 binding domain of an IL-2 receptor (IL-2R).

67. The protease-activated pro-cytokine of the immediately preceding claim, wherein the inhibitory polypeptide sequence comprises an amino acid sequence having at least 80, 85, 90, 95, 97, 98, or 99 percent identity to the sequence of any one of SEQ ID NOs: 10-29 or 40-51.

68. The protease-activated pro-cytokine of the immediately preceding claim, wherein the IL-2R is a human IL-2R.

69. The protease-activated pro-cytokine of any one of claims 60-65, wherein the inhibitory polypeptide sequence comprises an IL-2-binding immunoglobulin domain.

70. The protease-activated pro-cytokine of claim 69, wherein the IL-2-binding immunoglobulin domain is a human IL-2-binding immunoglobulin domain.

71. The protease-activated pro-cytokine of the immediately preceding claim, wherein the IL-2-binding immunoglobulin domain comprises a VL region comprising hypervariable regions (HVRs) HVR-1, HVR-2, and HVR-3 having the sequences of SEQ ID NOs: 33, 34, and 35, respectively, and a VH region comprising HVR-1, HVR-2, and HVR-3 having the sequences of SEQ ID NOs: 36, 37, and 38, respectively; or the IL-2-binding immunoglobulin domain comprises a VL region comprising hypervariable regions (HVRs) HVR-1, HVR-2, and HVR-3 having the sequences of SEQ ID NOs: 250, 251, and 252, respectively, and a VH region comprising HVR-1, HVR-2, and HVR-3 having the sequences of SEQ ID NOs: 253, 254, and 255, respectively.

72. The protease-activated pro-cytokine of any one of claims 69-71, wherein the IL-2-binding immunoglobulin domain comprises a VL region comprising an amino acid sequence having at least 80, 85, 90, 95, 97, 98, or 99 percent identity to the sequence of SEQ ID NO: 32 and a VH region comprising an amino acid sequence having at least 80, 85, 90, 95, 97, 98, or 99 percent identity to the sequence of SEQ ID NO: 33; or the IL-2-binding immunoglobulin domain comprises a VL region comprising an amino acid sequence having at least 80, 85, 90, 95, 97, 98, or 99 percent identity to the sequence of SEQ ID NO: 249 and a VH region comprising an amino acid sequence having at least 80, 85, 90, 95, 97, 98, or 99 percent identity to the sequence of SEQ ID NO: 248.

73. The protease-activated pro-cytokine of the immediately preceding claim, wherein the IL-2-binding immunoglobulin domain comprises a VL region comprising the sequence of SEQ ID NO: 32 and a VH region comprising the sequence of SEQ ID NO: 33; or the IL-2-binding immunoglobulin domain comprises a VL region comprising the sequence of SEQ ID NO: 249 and a VH region comprising the sequence of SEQ ID NO: 248.

74. The protease-activated pro-cytokine of any one of claims 69-73, wherein the IL-2-binding immunoglobulin domain is an scFv.

75. The protease-activated pro-cytokine of the immediately preceding claim, wherein the IL-2-binding immunoglobulin domain comprises an amino acid sequence having at least 80, 85, 90, 95, 97, 98, or 99 percent identity to the sequence of SEQ ID NO: 30, 31, or 247.

76. The protease-activated pro-cytokine of the immediately preceding claim, wherein the IL-2-binding immunoglobulin domain comprises the sequence of SEQ ID NO: 30, 31, or 247.

77. A pharmaceutical composition comprising the protease-activated pro-cytokine of any one of the preceding claims.

78. The protease-activated pro-cytokine or pharmaceutical composition of any one of the preceding claims, for use in therapy.

79. The protease-activated pro-cytokine or pharmaceutical composition of any one of the preceding claims, for use in treating a cancer.

80. A method of treating a cancer, comprising administering the protease-activated pro-cytokine or pharmaceutical composition of any one of the preceding claims to a subject in need thereof.

81. Use of the protease-activated pro-cytokine or pharmaceutical composition of any one of claims 1-77 for the manufacture of a medicament for treating cancer.

82. A method of creating a cytokine gradient in a subject, comprising administering the protease-activated pro-cytokine or pharmaceutical composition of any one of claims 1-77 to a subject, wherein the subject comprises a site having an abnormally high level of a protease that cleaves the protease-cleavable polypeptide sequence, optionally wherein the site comprises a cancer.

83. The protease-activated pro-cytokine or pharmaceutical composition of any one of claims 1-77, for use in a method of creating a cytokine gradient in a subject, comprising administering the protease-activated pro-cytokine or pharmaceutical composition to a subject, wherein the subject comprises a site having an abnormally high level of a protease that cleaves the protease-cleavable polypeptide sequence, optionally wherein the site comprises a cancer.

84. Use of the protease-activated pro-cytokine or pharmaceutical composition of any one of claims 1-77 for the manufacture of a medicament for creating a cytokine gradient in a subject, comprising administering the protease-activated pro-cytokine or pharmaceutical composition to a subject, wherein the subject comprises a site having an abnormally high level of a protease that cleaves the protease-cleavable polypeptide sequence, optionally wherein the site comprises a cancer.

85. The method, use, or protease-activated pro-cytokine for use of any one of claims 79-84, wherein the cancer is a solid tumor.

86. The method, use, or protease-activated pro-cytokine for use of the immediately preceding claim, wherein the solid tumor is metastatic and/or unresectable.

87. The method, use, or protease-activated pro-cytokine for use of any one of claims 79-86, wherein the cancer is a PD-L1-expressing cancer.

88. The method, use, or protease-activated pro-cytokine for use of any one of claims 79-87, wherein the cancer is a melanoma, a colorectal cancer, a breast cancer, a pancreatic cancer, a lung cancer, a prostate cancer, an ovarian cancer, a cervical cancer, a gastric or gastrointestinal cancer, a lymphoma, a colon or colorectal cancer, an endometrial cancer, a thyroid cancer, or a bladder cancer.

89. The method, use, or protease-activated pro-cytokine for use of any one of claims 79-88, wherein the cancer is a microsatellite instability-high cancer.

90. The method, use, or protease-activated pro-cytokine for use of any one of claims 79-89, wherein the cancer is mismatch repair deficient.

91. A nucleic acid encoding the protease-activated pro-cytokine of any one of claims 1-76.

92. An expression vector comprising the nucleic acid of claim 91.

93. A host cell comprising the nucleic acid of claim 91 or the vector of claim 92.

94. A method of producing a protease-activated pro-cytokine, comprising culturing the host cell of claim 93 under conditions wherein the protease-activated pro-cytokine is produced.

95. The method of the immediately preceding claim, further comprising isolating the protease-activated pro-cytokine.

96. A method of boosting T regulatory cells and/or reducing inflammation or autoimmune activity, comprising administering the protease-activated pro-cytokine of any one of claims 1-77 to an area of interest in a subject, e.g., an area of inflammation in the subject.

97. A method of treating an inflammatory or autoimmune disease or disorder in a subject, comprising administering the protease-activated pro-cytokine of any one of claims 1-77 to an area of interest in a subject, e.g., an area of inflammation or autoimmune activity in the subject.


SEQUENCE LISTING (257 sequences)

SEQ ID NO: 1 - protein, 133 aa, Homo sapiens [sequence omitted]

SEQ ID NO: 2 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 149 aa, Mus musculus
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 4 - protein, 149 aa, Mus musculus
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFSQSIISTSPQ

SEQ ID NO: 5-9 - skipped (5 entries)

SEQ ID NO: 10 - protein, 219 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 11 - protein, 63 aa, Homo sapiens
ELCDDDPPEIPHATFKAMAYKEGTMLNCECKRGFRRIKSGSLYMLCTGNSSHSSWDNQCQCTS

SEQ ID NO: 12 - protein, 219 aa, Homo sapiens
ELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 13 - protein, 219 aa, Homo sapiens
ELCDDDPPEIPHATFKAMAYKEGTMLNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 14 - protein, 219 aa, Homo sapiens
ELCDDDPPEIPHATFKAMAYKEGTILNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 15 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 16 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 17 - protein, 214 aa, Artificial sequence
  Homo sapiens / Mus musculus
ELCLYDPPEVPNATFKALSYKNGTILNCECKRGFRRLKELVYMRCLGNSWSSNCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 18 - protein, 215 aa, Mus musculus
ELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 19 - protein, 58 aa, Mus musculus
ELCLYDPPEVPNATFKALSYKNGTILNCECKRGFRRLKELVYMRCLGNSWSSNCQCTS

SEQ ID NO: 20 - protein, 219 aa, Homo sapiens
ELCLYDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 21 - protein, 219 aa, Homo sapiens
ELCLYDPPEIPHATFKAMAYKEGTMLNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 22 - protein, 219 aa, Homo sapiens
ELCLYDPPEIPHATFKAMAYKEGTILNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 23 - protein, 219 aa, Homo sapiens
ELCLYDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 24 - protein, 218 aa, Homo sapiens
ELCDDDPPEIPHATFKAMAYKEGTMLNCECKRGFRRIKELVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 25 - protein, 192 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 26 - protein, 192 aa, Homo sapiens
ELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 27 - protein, 192 aa, Homo sapiens
ELCDDDPPEIPHATFKAMAYKEGTMLNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 28 - protein, 192 aa, Homo sapiens
ELCLYDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 29 - protein, 191 aa, Homo sapiens
ELCDDDPPEIPHATFKAMAYKEGTMLNCECKRGFRRIKELVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 30 - protein, 247 aa, Homo sapiens
QSVLTQPPSVSGAPGQRVTISCTGTSSNIGAHYDVHWYQQFPGTAPKRLIYGNNNRPSGVPARFSGSKSGTSASLAITGLQAEDEADYYCQSYDRSLRGWVFGGGTKLTVLGEGKSSGSGSESKASEVQLVESGGGLVQPGRSLRLSCAASGFTFDDYAMHWVRQAPGKGLEWVSGISWNSGSIGYADSVKGRFTISRDNSKNTLYLQMNSLRAEDTAVYYCAKDVNWNYGYYFDYWGQGTLVTVSS

SEQ ID NO: 31 - protein, 251 aa, Homo sapiens
QSVLTQPPSVSGAPGQRVTISCTGTSSNIGAHYDVHWYQQFPGTAPKRLIYGNNNRPSGVPARFSGSKSGTSASLAITGLQAEDEADYYCQSYDRSLRGWVFGGGTKLTVLGGSTSGSGKPGSGEGSTKGEVQLVESGGGLVQPGRSLRLSCAASGFTFDDYAMHWVRQAPGKGLEWVSGISWNSGSIGYADSVKGRFTISRDNSKNTLYLQMNSLRAEDTAVYYCAKDVNWNYGYYFDYWGQGTLVTVSS

SEQ ID NO: 32 - protein, 112 aa, Homo sapiens
QSVLTQPPSVSGAPGQRVTISCTGTSSNIGAHYDVHWYQQFPGTAPKRLIYGNNNRPSGVPARFSGSKSGTSASLAITGLQAEDEADYYCQSYDRSLRGWVFGGGTKLTVLG

SEQ ID NO: 33 - protein, 121 aa, Homo sapiens
EVQLVESGGGLVQPGRSLRLSCAASGFTFDDYAMHWVRQAPGKGLEWVSGISWNSGSIGYADSVKGRFTISRDNSKNTLYLQMNSLRAEDTAVYYCAKDVNWNYGYYFDYWGQGTLVTVSS

SEQ ID NO: 34 - protein, 14 aa, Artificial sequence
  scFv2 VL HVR1
TGTSSNIGAHYDVH

SEQ ID NO: 35 - protein, 7 aa, Artificial sequence
  scFv2 VL HVR2
GNNNRPS

SEQ ID NO: 36 - protein, 11 aa, Artificial sequence
  scFv2 VL HVR3
QSYDRSLRGWV

SEQ ID NO: 37 - protein, 6 aa, Artificial sequence
  scFv2 VH HVR1
DDYAMH

SEQ ID NO: 38 - protein, 17 aa, Artificial sequence
  scFv2 VH HVR2
GISWNSGSIGYADSVKG

SEQ ID NO: 39 - protein, 13 aa, Artificial sequence
  scFv2 VH HVR3
KDVNWNYGYYFDY

SEQ ID NO: 40 - protein, 192 aa, Homo sapiens
ELCLYDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 41 - protein, 192 aa, Homo sapiens
ELCDDDPPEIPHATFKAMAYKEGTILNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 42 - protein, 192 aa, Homo sapiens
ELCLYDPPEIPHATFKAMAYKEGTMLNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 43 - protein, 192 aa, Homo sapiens
ELCLYDPPEIPHATFKAMAYKEGTILNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSC

SEQ ID NO: 44 - protein, 178 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 45 - protein, 178 aa, Homo sapiens
ELCDDDPPEIPHATFKAMAYKEGTILNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 46 - protein, 178 aa, Homo sapiens
ELCDDDPPEIPHATFKAMAYKEGTMLNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 47 - protein, 178 aa, Homo sapiens
ELCLYDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 48 - protein, 177 aa, Homo sapiens
ELCDDDPPEIPHATFKAMAYKEGTMLNCECKRGFRRIKELVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 49 - protein, 178 aa, Homo sapiens
ELCDDDPPEIPHATFKAMAYKEGTILNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 50 - protein, 178 aa, Homo sapiens
ELCLYDPPEIPHATFKAMAYKEGTMLNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 51 - protein, 178 aa, Homo sapiens
ELCLYDPPEIPHATFKAMAYKEGTILNCECKRGFRRIKSGSVYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKP

SEQ ID NO: 52-69 - skipped (18 entries)

SEQ ID NO: 70 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 72 - protein, 591 aa, Homo sapiens
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 73 - protein, 222 aa, Mus musculus
GCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 74 - protein, 221 aa, Mus musculus
GCKPCICTVPEVSSVFIFPPKPKDVLMITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 75-79 - skipped (5 entries)

SEQ ID NO: 80 - protein, 7 aa, Artificial sequence
  MMP cleavage site polypeptide sequence
GPLGVRG

SEQ ID NO: 81 - protein, 7 aa, Artificial sequence
  G112631 polypeptide sequence
GPLGVRG

SEQ ID NO: 82 - protein, 7 aa, Artificial sequence
  G112632  polypeptide sequence
GPLGLRG

SEQ ID NO: 83 - protein, 7 aa, Artificial sequence
  G112633  polypeptide sequence
GPLGLAR

SEQ ID NO: 84 - protein, 8 aa, Artificial sequence
  G112634  polypeptide sequence
GPAALVGA

SEQ ID NO: 85 - protein, 8 aa, Artificial sequence
  G112635  polypeptide sequence
GPAALIGG

SEQ ID NO: 86 - protein, 8 aa, Artificial sequence
  G112636  polypeptide sequence
GPLNLVGR

SEQ ID NO: 87 - protein, 8 aa, Artificial sequence
  G112637  polypeptide sequence
GPAGLVAD

SEQ ID NO: 88 - protein, 8 aa, Artificial sequence
  G112638  polypeptide sequence
GPANLVAP

SEQ ID NO: 89 - protein, 8 aa, Artificial sequence
  G112639  polypeptide sequence
VPLSLYSG

SEQ ID NO: 90 - protein, 10 aa, Artificial sequence
  G112640  polypeptide sequence
SGESPAYYTA

SEQ ID NO: 91 - protein, 4 aa, Artificial sequence
  MMP consensus motif
  MISC_FEATURE (2)..(3): X = Any amino acid
  MISC_FEATURE (4)..(4): X = Hydrophobic amino acid
PXXX

SEQ ID NO: 92 - protein, 4 aa, Artificial sequence
  MMP-2 consensus motif
  MISC_FEATURE (1)..(1): X = L or I
  MISC_FEATURE (2)..(3): X = Any amino acid
  MISC_FEATURE (4)..(4): X = Hydrophobic amino acid
XXXX

SEQ ID NO: 93 - protein, 4 aa, Artificial sequence
  MMP-2 consensus motif
  MISC_FEATURE (1)..(1): X = Hydrophobic amino acid
  MISC_FEATURE (3)..(3): X = Any amino acid
XSXL

SEQ ID NO: 94 - protein, 4 aa, Artificial sequence
  MMP-2 consensus motif
  MISC_FEATURE (2)..(3): X = Any amino acid
  MISC_FEATURE (4)..(4): X = Hydrophobic amino acid
HXXX

SEQ ID NO: 95-99 - skipped (5 entries)

SEQ ID NO: 100 - protein, 397 aa, Artificial sequence
  TB001 polypeptide sequence: mIL2- 2x(SG4) - MMPcs1 - 2x(G4S) - IL2Ralpha - 6His
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKHHHHHH

SEQ ID NO: 101 - protein, 613 aa, Artificial sequence
  TB002 polypeptide sequence: m IL2-2x(SG4) - MMPcs1 - 2x (G4S) - IL2Ralpha - mIgG1 Fc
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 102 - protein, 618 aa, Artificial sequence
  TB003 polypeptide sequence: mIL2(C140S) - 2x(SG4) - MMPcs1 - 2x(G4S) - IL2Ralpha - mIgG1 Fc(T252M)- 6xHIS
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFSQSIISTSPQSGGGGSGGGGGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKGCKPCICTVPEVSSVFIFPPKPKDVLMITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGHHHHHH

SEQ ID NO: 103 - protein, 623 aa, Artificial sequence
  TB004 polypeptide sequence: mIL2(C140S)- 2x(SG4) - MMPcs1 - 2x(G4S) - IL2Ralpha - hu IgG1 Fc- 6xHIS
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFSQSIISTSPQSGGGGSGGGGGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 104 - protein, 611 aa, Artificial sequence
  TB005 polypeptide Sequence: mIL2(C140S)- 2x(SG4) - MMPcs1 - 2x(G4S) - sIL2Ralpha - mIgG1 Fc(T252M)- 6xHIS
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGGGSGGGGGPLGVRGGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 105 - protein, 613 aa, Artificial sequence
  mIgG1 Fc - Murine IL2 -2x(SG4) - MMPcs1 - 2x (G4S) - IL2Ralpha (long kinetic IL2 post cleavage) polypeptide Sequence
GCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSGGGGSGGGGGPLGVRGGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 106 - protein, 611 aa, Artificial sequence; identical to SEQ ID NO: 104
  TB006 polypeptide sequence: HuIL2(C125S)- 2x(SG4) - MMPcs1 - 2x(G4S) - IL2Ralpha - hu IgG1 Fc - 6xHIS

SEQ ID NO: 107 - protein, 611 aa, Artificial sequence
  TB007a polypeptide sequence: hIL2- 2x(SG4) - MMPcs1 - 2x(G4S) - hIL2Ralpha - hIgG1Fc_mut1 (K392D; K409D)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGGGSGGGGGPLGVRGGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGHHHHHH

SEQ ID NO: 108 - protein, 232 aa, Artificial sequence
  TB007b polypeptide sequence including hIgG1 Fc_mut 2 (D356K; D399K)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGHHHHHH

SEQ ID NO: 109 - protein, 605 aa, Artificial sequence
  hu IgG1 Fc  - Hu IL2(C125S)- 2x(SG4) - MMPcs1 - 2x(G4S) - IL2Ralpha Polypeptide Sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGGGSGGGGGPLGVRGGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 110 - protein, 600 aa, Artificial sequence
  hIL2- 2x(SG4) - MMPcs1 - 2x(G4S) - hIL2Rbeta - hIgG1Fc (TB010) polypeptide Sequence
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGGGSGGGGGPLGVRGGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 626 aa, Artificial sequence
  hIL2- 2x(SG4) - MMPcs1 - 2x(G4S) - hIL2Rgamma - hIgG1Fc polypeptide sequence
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGGGSGGGGGPLGVRGGGGGSGGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 112-119 - skipped (8 entries)

SEQ ID NO: 120 - protein, 10 aa, Artificial sequence
  Gly-Ser rich linker polypeptide sequence
SGGGGSGGGG

SEQ ID NO: 121-129 - skipped (9 entries)

SEQ ID NO: 130 - DNA, 1254 bp, Artificial sequence
  TB001 DNA sequence (mIL2- 2x(SG4) - MMPcs1 - 2x(G4S) - IL2Ralpha - 6His)
atgggttggtcctgcatcatcctgttcctggtcgccaccgccactggggtccactccgcacctacatcatcatcaacttcatcctccaccgctgaggctcagcaacaacagcaacaacagcagcagcagcagcagcatctggagcagctgctgatggacctgcaggagctgctgtccagaatggagaactaccgcaatctgaagctgccaaggatgctgaccttcaagttttatctgcccaagcaggccacagagctgaaggacctgcagtgcctggaggatgagctgggcccactgaggcacgtgctggacctgacccagagcaagtctttccagctggaggatgctgagaactttatctccaatatccgggtgaccgtggtgaagctgaagggcagcgacaacacattcgagtgccagtttgacgatgagtctgccaccgtggtggatttcctgaggcggtggatcgctttttgtcagagcatcatctccacaagccctcagtctggaggaggtggcagcggaggaggaggtggcccactgggcgtgaggggtggcggcggcggctctggcggcggcggctccgagctgtgcctgtacgacccccctgaggtgcccaatgccaccttcaaggctctgtcttataagaacggcacaatcctgaattgcgagtgtaagaggggctttagacgcctgaaggagctggtgtacatgcggtgtctgggcaactcctggtccagcaattgccagtgtacctctaactcccatgacaagagcagaaagcaggtgacagcccagctggagcaccagaaggagcagcagaccacaaccgatatgcagaagcccacccagtctatgcaccaggagaatctgacaggccattgcagagagccacccccttggaagcacgaggatagcaagcgcatctatcatttcgtggagggccagtctgtgcactacgagtgtatccccggctataaggccctgcagagaggccctgctatctccatctgcaagatgaagtgtggcaagaccggctggacacagcctcagctgacctgcgtggacgagagggagcaccatcggttcctggctagcgaggagtctcagggctcccgcaactcttcccctgagagcgagacatcttgtccaatcacaaccacagattttccacagcccaccgagacaaccgctatgacagagaccttcgtgctgactatggaatacaaacaccaccaccaccaccactaatga

SEQ ID NO: 131 - DNA, 1902 bp, Artificial sequence
  TB002 DNA sequence: m IL2-2x(SG4) - MMPcs1 - 2x (G4S) - IL2Ralpha - mIgG1 Fc
atgggttggtcctgcatcatcctgttcctggtcgccaccgccactggggtccactccgcacctacatcatcatcaacttcatcctccaccgctgaggctcagcaacaacagcaacaacagcagcagcagcagcagcatctggagcagctgctgatggacctgcaggagctgctgtccagaatggagaactaccgcaatctgaagctgccaaggatgctgaccttcaagttttatctgcccaagcaggccacagagctgaaggacctgcagtgcctggaggatgagctgggcccactgaggcacgtgctggacctgacccagagcaagtctttccagctggaggatgctgagaactttatctccaatatccgggtgaccgtggtgaagctgaagggcagcgacaacacattcgagtgccagtttgacgatgagtctgccaccgtggtggatttcctgaggcggtggatcgctttttgtcagagcatcatctccacaagccctcagtctggaggaggtggcagcggaggaggaggtggcccactgggcgtgaggggtggcggcggcggctctggcggcggcggctccgagctgtgcctgtacgacccccctgaggtgcccaatgccaccttcaaggctctgtcttataagaacggcacaatcctgaattgcgagtgtaagaggggctttagacgcctgaaggagctggtgtacatgcggtgtctgggcaactcctggtccagcaattgccagtgtacctctaactcccatgacaagagcagaaagcaggtgacagcccagctggagcaccagaaggagcagcagaccacaaccgatatgcagaagcccacccagtctatgcaccaggagaatctgacaggccattgcagagagccacccccttggaagcacgaggatagcaagcgcatctatcatttcgtggagggccagtctgtgcactacgagtgtatccccggctataaggccctgcagagaggccctgctatctccatctgcaagatgaagtgtggcaagaccggctggacacagcctcagctgacctgcgtggacgagagggagcaccatcggttcctggctagcgaggagtctcagggctcccgcaactcttcccctgagagcgagacatcttgtccaatcacaaccacagattttccacagcccaccgagacaaccgctatgacagagaccttcgtgctgactatggaatacaaaggatgcaaaccctgtatctgtaccgtgcccgaggtctcttccgtctttattttcccccccaagcctaaggatgtgctgactattactctgacccccaaggtgacatgcgtggtggtggacatcagcaaggacgatcctgaggtgcagttctcttggtttgtggacgatgtggaggtgcacaccgcccagacacagccaagggaggagcagttcaatagcacctttcggtccgtgagcgagctgcccatcatgcatcaggattggctgaatggcaaggagttcaagtgcagagtgaactctgccgcttttcccgctcctatcgagaagaccatctccaagacaaagggccgcccaaaggctccacaggtgtacaccatcccacctccaaaggagcagatggctaaggacaaggtgtctctgacctgtatgatcacagacttctttcctgaggacatcacagtggagtggcagtggaacggccagcctgccgagaactataagaatacccagccaatcatggacacagatggctcttacttcgtgtattccaagctgaacgtgcagaagtccaattgggaggctggcaacacctttacatgtagcgtgctgcacgaaggtctgcataaccatcataccgaaaaatcactgtcacactcccctggaaaataatga

SEQ ID NO: 132 - DNA, 1917 bp, Artificial sequence
  TB003 DNA sequence: m IL2(C140S)- 2x(SG4) - MMPcs1 - 2x(G4S) - IL2Ralpha - mIgG1 Fc(T252M)- 6xHIS
atgggttggtcctgcatcatcctgttcctggtcgccaccgccactggggtccactccgcacctacatcatcatcaacttcatcctccaccgctgaggctcagcaacaacagcaacaacagcagcagcagcagcagcatctggagcagctgctgatggacctgcaggagctgctgtccagaatggagaactaccgcaatctgaagctgccaaggatgctgaccttcaagttttatctgcccaagcaggccacagagctgaaggacctgcagtgcctggaggatgagctgggcccactgaggcacgtgctggacctgacccagagcaagtctttccagctggaggatgctgagaactttatctccaatatccgggtgaccgtggtgaagctgaagggcagcgacaacacattcgagtgccagtttgacgatgagtctgccaccgtggtggatttcctgaggcggtggatcgctttttcccagagcatcatctccacaagccctcagtctggaggaggtggcagcggaggaggaggtggcccactgggcgtgaggggtggcggcggcggctctggcggcggcggctccgagctgtgcctgtacgacccccctgaggtgcccaatgccaccttcaaggctctgtcttataagaacggcacaatcctgaattgcgagtgtaagaggggctttagacgcctgaaggagctggtgtacatgcggtgtctgggcaactcctggtccagcaattgccagtgtacctctaactcccatgacaagagcagaaagcaggtgacagcccagctggagcaccagaaggagcagcagaccacaaccgatatgcagaagcccacccagtctatgcaccaggagaatctgacaggccattgcagagagccacccccttggaagcacgaggatagcaagcgcatctatcatttcgtggagggccagtctgtgcactacgagtgtatccccggctataaggccctgcagagaggccctgctatctccatctgcaagatgaagtgtggcaagaccggctggacacagcctcagctgacctgcgtggacgagagggagcaccatcggttcctggctagcgaggagtctcagggctcccgcaactcttcccctgagagcgagacatcttgtccaatcacaaccacagattttccacagcccaccgagacaaccgctatgacagagaccttcgtgctgactatggaatacaaaggatgcaaaccctgtatctgtaccgtgcccgaggtctcttccgtctttattttcccccccaagcctaaggatgtgctgatgattactctgacccccaaggtgacatgcgtggtggtggacatcagcaaggacgatcctgaggtgcagttctcttggtttgtggacgatgtggaggtgcacaccgcccagacacagccaagggaggagcagttcaatagcacctttcggtccgtgagcgagctgcccatcatgcatcaggattggctgaatggcaaggagttcaagtgcagagtgaactctgccgcttttcccgctcctatcgagaagaccatctccaagacaaagggccgcccaaaggctccacaggtgtacaccatcccacctccaaaggagcagatggctaaggacaaggtgtctctgacctgtatgatcacagacttctttcctgaggacatcacagtggagtggcagtggaacggccagcctgccgagaactataagaatacccagccaatcatggacacagatggctcttacttcgtgtattccaagctgaacgtgcagaagtccaattgggaggctggcaacacctttacatgtagcgtgctgcacgaaggtctgcataaccatcataccgaaaaatcactgtcacactcccctggacaccaccaccaccaccactaatga

SEQ ID NO: 133 - DNA, 1932 bp, Artificial sequence
  TB004 DNA sequence: mIL2(C140S)- 2x(SG4) - MMPcs1 - 2x(G4S) - IL2Ralpha - hu IgG1 Fc- 6xHIS
atgggttggtcctgcatcatcctgttcctggtcgccaccgccactggggtccactccgcacctacatcatcatcaacttcatcctccaccgctgaggctcagcaacaacagcaacaacagcagcagcagcagcagcatctggagcagctgctgatggacctgcaggagctgctgtccagaatggagaactaccgcaatctgaagctgccaaggatgctgaccttcaagttttatctgcccaagcaggccacagagctgaaggacctgcagtgcctggaggatgagctgggcccactgaggcacgtgctggacctgacccagagcaagtctttccagctggaggatgctgagaactttatctccaatatccgggtgaccgtggtgaagctgaagggcagcgacaacacattcgagtgccagtttgacgatgagtctgccaccgtggtggatttcctgaggcggtggatcgctttttcccagagcatcatctccacaagccctcagtctggaggaggtggcagcggaggaggaggtggcccactgggcgtgaggggtggcggcggcggctctggcggcggcggctccgagctgtgcctgtacgacccccctgaggtgcccaatgccaccttcaaggctctgtcttataagaacggcacaatcctgaattgcgagtgtaagaggggctttagacgcctgaaggagctggtgtacatgcggtgtctgggcaactcctggtccagcaattgccagtgtacctctaactcccatgacaagagcagaaagcaggtgacagcccagctggagcaccagaaggagcagcagaccacaaccgatatgcagaagcccacccagtctatgcaccaggagaatctgacaggccattgcagagagccacccccttggaagcacgaggatagcaagcgcatctatcatttcgtggagggccagtctgtgcactacgagtgtatccccggctataaggccctgcagagaggccctgctatctccatctgcaagatgaagtgtggcaagaccggctggacacagcctcagctgacctgcgtggacgagagggagcaccatcggttcctggctagcgaggagtctcagggctcccgcaactcttcccctgagagcgagacatcttgtccaatcacaaccacagattttccacagcccaccgagacaaccgctatgacagagaccttcgtgctgactatggaatacaaagataagactcatacctgtccaccctgtcctgctcctgaactgctgggcggtccttccgtgttcctgttccctccaaaacctaaagataccctgatgatctccaggacccctgaggtgacatgcgtggtggtggacgtgagccacgaggaccccgaggtgaagttcaactggtacgtggacggcgtggaggtgcataatgccaagacaaagcccagggaggagcagtacaacagcacctatcgggtggtgtctgtgctgacagtgctgcaccaggattggctgaacggcaaggagtataagtgcaaggtgtctaataaggccctgcctgctccaatcgagaagaccatctccaaggccaagggccagcccagagagcctcaggtgtacacactgccccctagccgcgacgagctgaccaagaaccaggtgtctctgacatgtctggtgaagggcttctatccatctgacatcgctgtggagtgggagtccaatggccagcccgagaacaattacaagaccacaccacccgtgctggactctgatggctccttctttctgtattccaagctgaccgtggacaagagcagatggcagcagggcaacgtgttctcctgtagcgtgatgcacgaagccctgcacaaccattacactcagaaaagcctgtccctgtcccctgggcaccaccaccaccaccactaatga

SEQ ID NO: 134 - DNA, 1794 bp, Artificial sequence
  TB005 DNA Sequence: mIL2(C140S)- 2x(SG4) - MMPcs1 - 2x(G4S) - sIL2Ralpha - mIgG1 Fc(T252M)- 6xHIS
atgggttggtcctgcatcatcctgttcctggtcgccaccgccactggggtccactccgcacctacatcatcatcaacttcatcctccaccgctgaggctcagcaacaacagcaacaacagcagcagcagcagcagcatctggagcagctgctgatggacctgcaggagctgctgtccagaatggagaactaccgcaatctgaagctgccaaggatgctgaccttcaagttttatctgcccaagcaggccacagagctgaaggacctgcagtgcctggaggatgagctgggcccactgaggcacgtgctggacctgacccagagcaagtctttccagctggaggatgctgagaactttatctccaatatccgggtgaccgtggtgaagctgaagggcagcgacaacacattcgagtgccagtttgacgatgagtctgccaccgtggtggatttcctgaggcggtggatcgctttttcccagagcatcatctccacaagccctcagtctggaggaggtggcagcggaggaggaggtggcccactgggcgtgaggggtggcggcggcggctctggcggcggcggctccgagctgtgcctgtacgacccccctgaggtgcccaatgccaccttcaaggctctgtcttataagaacggcacaatcctgaattgcgagtgtaagaggggctttagacgcctgaaggagctggtgtacatgcggtgtctgggcaactcctggtccagcaattgccagtgtacctctaactcccatgacaagagcagaaagcaggtgacagcccagctggagcaccagaaggagcagcagaccacaaccgatatgcagaagcccacccagtctatgcaccaggagaatctgacaggccattgcagagagccacccccttggaagcacgaggatagcaagcgcatctatcatttcgtggagggccagtctgtgcactacgagtgtatccccggctataaggccctgcagagaggccctgctatctccatctgcaagatgaagtgtggcaagaccggctggacacagcctcagctgacctgcgtggacgagagggagcaccatcggttcctggctagcgaggagtctggatgcaaaccctgtatctgtaccgtgcccgaggtctcttccgtctttattttcccccccaagcctaaggatgtgctgatgattactctgacccccaaggtgacatgcgtggtggtggacatcagcaaggacgatcctgaggtgcagttctcttggtttgtggacgatgtggaggtgcacaccgcccagacacagccaagggaggagcagttcaatagcacctttcggtccgtgagcgagctgcccatcatgcatcaggattggctgaatggcaaggagttcaagtgcagagtgaactctgccgcttttcccgctcctatcgagaagaccatctccaagacaaagggccgcccaaaggctccacaggtgtacaccatcccacctccaaaggagcagatggctaaggacaaggtgtctctgacctgtatgatcacagacttctttcctgaggacatcacagtggagtggcagtggaacggccagcctgccgagaactataagaatacccagccaatcatggacacagatggctcttacttcgtgtattccaagctgaacgtgcagaagtccaattgggaggctggcaacacctttacatgtagcgtgctgcacgaaggtctgcataaccatcataccgaaaaatcactgtcacactcccctggacaccaccaccaccaccactaatga

SEQ ID NO: 135 - DNA, 1896 bp, Artificial sequence
  TB006 DNA sequence: HuIL2(C125S)- 2x(SG4) - MMPcs1 - 2x(G4S) - IL2Ralpha - hu IgG1 Fc - 6xHIS
atgggctggtcctgcatcattctgtttctggtggctaccgccaccggcgtgcactctgctcctacatcctccagcaccaagaaaacccagctgcagttggagcatctgctgctggacctgcagatgatcctgaacggcatcaacaactacaagaaccccaagctgacccggatgctgaccttcaagttctacatgcccaagaaggccaccgagctgaaacatctgcagtgcctggaagaggaactgaagcccctggaagaagtgctgaatctggcccagtccaagaacttccacctgaggcctcgggacctgatctccaacatcaacgtgatcgtgctcgagctgaagggctccgagacaaccttcatgtgcgagtacgccgacgagacagctaccatcgtggaatttctgaaccggtggatcaccttcagccagtccatcatcagcaccctgacatctggcggcggaggatctggcggaggcggaggacctttgggagttcgcggcggtggtggtggcagcggaggtggtggatctgagctgtgtgacgacgaccctcctgagatccctcacgccacctttaaggccatggcttacaaagagggcaccatgctgaactgcgagtgcaagagaggcttccggcggatcaagtccggcagcctgtatatgctgtgcaccggcaactccagccactcctcttgggacaaccagtgccagtgcaccagctctgctacccggaacaccaccaagcaagtgacccctcagcctgaggaacagaaagagcgcaagaccaccgagatgcagagccccatgcagcctgtggatcaggcttctctgcctggccactgtagagagcctccaccttgggagaatgaggctaccgagagaatctaccacttcgtcgtgggacagatggtgtactaccagtgcgtgcagggctaccgcgctctgcatagaggaccagcagagtccgtgtgcaagatgacccacggcaagaccagatggacccagcctcagctgatctgcaccggcgagatggaaacctctcagttccccggcgaggaaaagcctcaggcctctcctgaaggcagacccgagtctgagacatcctgtctcgtgaccaccacagacttccagatccagaccgagatggccgctaccatggaaaccagcatcttcaccaccgagtaccaggacaagacccacacctgtcctccatgtcctgctccagaattgctcggcggaccctccgtgttcctgtttcctccaaagcctaaggacaccctgatgatctctcggacccctgaagtgacctgcgtggtggtcgatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaatggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctggtcaagggcttctacccctccgatatcgccgtggaatgggagtctaatggccagcctgaaaacaattacaagacaacccctcctgtgctggactccgacggctcattcttcctgtacagcaagctgacagtggacaagtccagatggcagcagggcaacgtgttctcctgctccgtgatgcatgaggccctgcacaaccactacacccagaagtccctgtctctgtcccctggccaccatcaccatcatcactgataa

SEQ ID NO: 136 - DNA, 1896 bp, Artificial sequence
  TB007a DNA sequence: hIL2- 2x(SG4) - MMPcs1 - 2x(G4S) - hIL2Ralpha - hIgG1Fc_mut1 (K392D; K409D)
atgggttggtcctgcatcatcctgttcctggtcgccaccgccactggggtccactccgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttctggtggcggtggctctggtggcggtggcggtcctctgggtgtcagaggtggtggcggtggctctggtggcggtggctctgagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtcaccaccaccaccaccactaatga

SEQ ID NO: 137 - DNA, 759 bp, Artificial sequence
  TB007b DNA sequence including hIgG1 Fc_mut 2 (D356K; D399K)
atgggttggtcctgcatcatcctgttcctggtcgccaccgccactggggtccactccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaagagctgaccaagaaccaggtcagcctgacctgcctggtcaaaggcttctatcccagcgacatcgccgtggagtgggagagcaatgggcagccggagaacaactacaagaccacgcctcccgtgctgaaatccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtcaccaccaccaccaccactaatga

SEQ ID NO: 138 - DNA, 1902 bp, Artificial sequence
  mIgG1 Fc - Murine IL2 -2x(SG4) - MMPcs1 - 2x (G4S) - IL2Ralpha (long kinetic IL2 post cleavage) DNA Sequence
atgggttggtcctgcatcatcctgttcctggtcgccaccgccactggggtccactccggatgcaaaccctgtatctgtaccgtgcccgaggtctcttccgtctttattttcccccccaagcctaaggatgtgctgactattactctgacccccaaggtgacatgcgtggtggtggacatcagcaaggacgatcctgaggtgcagttctcttggtttgtggacgatgtggaggtgcacaccgcccagacacagccaagggaggagcagttcaatagcacctttcggtccgtgagcgagctgcccatcatgcatcaggattggctgaatggcaaggagttcaagtgcagagtgaactctgccgcttttcccgctcctatcgagaagaccatctccaagacaaagggccgcccaaaggctccacaggtgtacaccatcccacctccaaaggagcagatggctaaggacaaggtgtctctgacctgtatgatcacagacttctttcctgaggacatcacagtggagtggcagtggaacggccagcctgccgagaactataagaatacccagccaatcatggacacagatggctcttacttcgtgtattccaagctgaacgtgcagaagtccaattgggaggctggcaacacctttacatgtagcgtgctgcacgaaggtctgcataaccatcataccgaaaaatcactgtcacactcccctggaaaagcacctacatcatcatcaacttcatcctccaccgctgaggctcagcaacaacagcaacaacagcagcagcagcagcagcatctggagcagctgctgatggacctgcaggagctgctgtccagaatggagaactaccgcaatctgaagctgccaaggatgctgaccttcaagttttatctgcccaagcaggccacagagctgaaggacctgcagtgcctggaggatgagctgggcccactgaggcacgtgctggacctgacccagagcaagtctttccagctggaggatgctgagaactttatctccaatatccgggtgaccgtggtgaagctgaagggcagcgacaacacattcgagtgccagtttgacgatgagtctgccaccgtggtggatttcctgaggcggtggatcgctttttgtcagagcatcatctccacaagccctcagtctggaggaggtggcagcggaggaggaggtggcccactgggcgtgaggggtggcggcggcggctctggcggcggcggctccgagctgtgcctgtacgacccccctgaggtgcccaatgccaccttcaaggctctgtcttataagaacggcacaatcctgaattgcgagtgtaagaggggctttagacgcctgaaggagctggtgtacatgcggtgtctgggcaactcctggtccagcaattgccagtgtacctctaactcccatgacaagagcagaaagcaggtgacagcccagctggagcaccagaaggagcagcagaccacaaccgatatgcagaagcccacccagtctatgcaccaggagaatctgacaggccattgcagagagccacccccttggaagcacgaggatagcaagcgcatctatcatttcgtggagggccagtctgtgcactacgagtgtatccccggctataaggccctgcagagaggccctgctatctccatctgcaagatgaagtgtggcaagaccggctggacacagcctcagctgacctgcgtggacgagagggagcaccatcggttcctggctagcgaggagtctcagggctcccgcaactcttcccctgagagcgagacatcttgtccaatcacaaccacagattttccacagcccaccgagacaaccgctatgacagagaccttcgtgctgactatggaatacaaataatga

SEQ ID NO: 139 - DNA, 1863 bp, Artificial sequence
  hIL2- 2x(SG4) - MMPcs1 - 2x(G4S) - hIL2Rbeta - hIgG1Fc (TB010) DNA Sequence
atgggttggtcctgcatcatcctgttcctggtcgccaccgccactggggtccactccgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttctggtggcggtggctctggtggcggtggcggtcctctgggtgtcagaggtggtggcggtggctctggtggcggtggctctgcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctcccactactttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggacaccgacaagacccacacctgtcctccatgtcctgctccagaattgctcggcggaccctccgtgttcctgtttcctccaaagcctaaggacaccctgatgatctctcggacccctgaagtgacctgcgtggtggtcgatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaatggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctggtcaagggcttctacccctccgatatcgccgtggaatgggagtctaatggccagcctgaaaacaattacaagacaacccctcctgtgctggactccgacggctcattcttcctgtacagcaagctgacagtggacaagtccagatggcagcagggcaacgtgttctcctgctccgtgatgcatgaggccctgcacaaccactacacccagaagtccctgtctctgtcccctggctaatga

SEQ ID NO: 140 - DNA, 1941 bp, Artificial sequence
  hIL2- 2x(SG4) - MMPcs1 - 2x(G4S) - hIL2Rgamma - hIgG1Fc DNA sequence
atgggttggtcctgcatcatcctgttcctggtcgccaccgccactggggtccactccgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttctggtggcggtggctctggtggcggtggcggtcctctgggtgtcagaggtggtggcggtggctctggtggcggtggctctctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgacaagacccacacctgtcctccatgtcctgctccagaattgctcggcggaccctccgtgttcctgtttcctccaaagcctaaggacaccctgatgatctctcggacccctgaagtgacctgcgtggtggtcgatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaatggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctggtcaagggcttctacccctccgatatcgccgtggaatgggagtctaatggccagcctgaaaacaattacaagacaacccctcctgtgctggactccgacggctcattcttcctgtacagcaagctgacagtggacaagtccagatggcagcagggcaacgtgttctcctgctccgtgatgcatgaggccctgcacaaccactacacccagaagtccctgtctctgtcccctggctaatga

SEQ ID NO: 141-149 - skipped (9 entries)

SEQ ID NO: 150 - DNA, 57 bp, Artificial sequence
  Murine Ig kappa chain leader DNA sequence
atgggttggtcctgcatcatcctgttcctggtcgccaccgccactggggtccactcc

SEQ ID NO: 151 - DNA, 447 bp, Artificial sequence
  Murine IL-2 DNA sequence
gcacctacatcatcatcaacttcatcctccaccgctgaggctcagcaacaacagcaacaacagcagcagcagcagcagcatctggagcagctgctgatggacctgcaggagctgctgtccagaatggagaactaccgcaatctgaagctgccaaggatgctgaccttcaagttttatctgcccaagcaggccacagagctgaaggacctgcagtgcctggaggatgagctgggcccactgaggcacgtgctggacctgacccagagcaagtctttccagctggaggatgctgagaactttatctccaatatccgggtgaccgtggtgaagctgaagggcagcgacaacacattcgagtgccagtttgacgatgagtctgccaccgtggtggatttcctgaggcggtggatcgctttttgtcagagcatcatctccacaagccctcag

SEQ ID NO: 152 - DNA, 21 bp, Artificial sequence
  MMP cleavage site GPLGVRG DNA sequence
ggcccactgggcgtgaggggt

SEQ ID NO: 153 - DNA, 30 bp, Artificial sequence
  Gly-Ser rich linker DNA sequence
tctggaggaggtggcagcggaggaggaggt

SEQ ID NO: 154 - DNA, 645 bp, Artificial sequence
  Murine IL-2Ralpha DNA sequence
gagctgtgcctgtacgacccccctgaggtgcccaatgccaccttcaaggctctgtcttataagaacggcacaatcctgaattgcgagtgtaagaggggctttagacgcctgaaggagctggtgtacatgcggtgtctgggcaactcctggtccagcaattgccagtgtacctctaactcccatgacaagagcagaaagcaggtgacagcccagctggagcaccagaaggagcagcagaccacaaccgatatgcagaagcccacccagtctatgcaccaggagaatctgacaggccattgcagagagccacccccttggaagcacgaggatagcaagcgcatctatcatttcgtggagggccagtctgtgcactacgagtgtatccccggctataaggccctgcagagaggccctgctatctccatctgcaagatgaagtgtggcaagaccggctggacacagcctcagctgacctgcgtggacgagagggagcaccatcggttcctggctagcgaggagtctcagggctcccgcaactcttcccctgagagcgagacatcttgtccaatcacaaccacagattttccacagcccaccgagacaaccgctatgacagagaccttcgtgctgactatggaatacaaa

SEQ ID NO: 155 - DNA, 18 bp, Artificial sequence
  His tag DNA Sequence
caccaccaccaccaccac

SEQ ID NO: 156 - DNA, 6 bp, Artificial sequence
  Stop codons
taatga

SEQ ID NO: 157 - DNA, 446 bp, Artificial sequence
  Murine IL-2 C140S DNA sequence
gcacctacatcatcatcaacttcatcctccaccgctgaggctcagcaacaacagcaacaacagcagcagcagcagcagcatctggagcagctgctgatggacctgcaggagctgctgtccagaatggagaactaccgcaatctgaagctgccaaggatgctgaccttcaagttttatctgcccaagcaggccacagagctgaaggacctgcagtgcctggaggatgagctgggcccactgaggcacgtgctggacctgacccagagcaagtctttccagctggaggatgctgagaactttatctccaatatccgggtgaccgtggtgaagctgaagggcagcgacaacacattcgagtgccagtttgacgatgagtctgccaccgtggtggatttcctgaggcggtggatcgctttttcccagagcatcatctccacaagccctca

SEQ ID NO: 158 - DNA, 663 bp, Artificial sequence
  Murine IgG1 T252M Fc domain DNA sequence
ggatgcaaaccctgtatctgtaccgtgcccgaggtctcttccgtctttattttcccccccaagcctaaggatgtgctgatgattactctgacccccaaggtgacatgcgtggtggtggacatcagcaaggacgatcctgaggtgcagttctcttggtttgtggacgatgtggaggtgcacaccgcccagacacagccaagggaggagcagttcaatagcacctttcggtccgtgagcgagctgcccatcatgcatcaggattggctgaatggcaaggagttcaagtgcagagtgaactctgccgcttttcccgctcctatcgagaagaccatctccaagacaaagggccgcccaaaggctccacaggtgtacaccatcccacctccaaaggagcagatggctaaggacaaggtgtctctgacctgtatgatcacagacttctttcctgaggacatcacagtggagtggcagtggaacggccagcctgccgagaactataagaatacccagccaatcatggacacagatggctcttacttcgtgtattccaagctgaacgtgcagaagtccaattgggaggctggcaacacctttacatgtagcgtgctgcacgaaggtctgcataaccatcataccgaaaaatcactgtcacactcccctgga

SEQ ID NO: 159 - DNA, 666 bp, Artificial sequence
  Murine IgG1 Fc domain DNA sequence
ggatgcaaaccctgtatctgtaccgtgcccgaggtctcttccgtctttattttcccccccaagcctaaggatgtgctgactattactctgacccccaaggtgacatgcgtggtggtggacatcagcaaggacgatcctgaggtgcagttctcttggtttgtggacgatgtggaggtgcacaccgcccagacacagccaagggaggagcagttcaatagcacctttcggtccgtgagcgagctgcccatcatgcatcaggattggctgaatggcaaggagttcaagtgcagagtgaactctgccgcttttcccgctcctatcgagaagaccatctccaagacaaagggccgcccaaaggctccacaggtgtacaccatcccacctccaaaggagcagatggctaaggacaaggtgtctctgacctgtatgatcacagacttctttcctgaggacatcacagtggagtggcagtggaacggccagcctgccgagaactataagaatacccagccaatcatggacacagatggctcttacttcgtgtattccaagctgaacgtgcagaagtccaattgggaggctggcaacacctttacatgtagcgtgctgcacgaaggtctgcataaccatcataccgaaaaatcactgtcacactcccctggaaaa

SEQ ID NO: 160 - DNA, 399 bp, Artificial sequence
  Human IL-2 C125S DNA sequence
gctcctacatcctccagcaccaagaaaacccagctgcagttggagcatctgctgctggacctgcagatgatcctgaacggcatcaacaactacaagaaccccaagctgacccggatgctgaccttcaagttctacatgcccaagaaggccaccgagctgaaacatctgcagtgcctggaagaggaactgaagcccctggaagaagtgctgaatctggcccagtccaagaacttccacctgaggcctcgggacctgatctccaacatcaacgtgatcgtgctcgagctgaagggctccgagacaaccttcatgtgcgagtacgccgacgagacagctaccatcgtggaatttctgaaccggtggatcaccttcagccagtccatcatcagcaccctgaca

SEQ ID NO: 161 - DNA, 678 bp, Artificial sequence
  Human IgG1 Fc domain DNA sequence
gacaagacccacacctgtcctccatgtcctgctccagaattgctcggcggaccctccgtgttcctgtttcctccaaagcctaaggacaccctgatgatctctcggacccctgaagtgacctgcgtggtggtcgatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaatggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctggtcaagggcttctacccctccgatatcgccgtggaatgggagtctaatggccagcctgaaaacaattacaagacaacccctcctgtgctggactccgacggctcattcttcctgtacagcaagctgacagtggacaagtccagatggcagcagggcaacgtgttctcctgctccgtgatgcatgaggccctgcacaaccactacacccagaagtccctgtctctgtcccctggc

SEQ ID NO: 162 - DNA, 399 bp, Artificial sequence
  Human IL-2 DNA sequence
gcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 163 - DNA, 678 bp, Artificial sequence
  Human IgG1 K392D K409D Fc domain DNA sequence
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggt

SEQ ID NO: 164 - DNA, 657 bp, Artificial sequence
  Human IL-2Ralpha DNA Sequence
gagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccag

SEQ ID NO: 165 - DNA, 30 bp, Artificial sequence
  Gly-Ser Linker DNA sequence
tctggtggcggtggctctggtggcggtggc

SEQ ID NO: 166 - DNA, 21 bp, Artificial sequence
  Human MMP Cleavage Site DNA sequence
ggtcctctgggtgtcagaggt

SEQ ID NO: 167-200 - skipped (34 entries)

SEQ ID NO: 201 - protein, 8 aa, Artificial sequence
  MMP7-cleavable sequence
KRALGLPG

SEQ ID NO: 202 - protein, 40 aa, Artificial sequence
  MMP7-cleavable sequence
DEDEDEDEDEDEDEDERPLALWRSDRDRDRDRDRDRDRDR

SEQ ID NO: 203 - protein, 5 aa, Artificial sequence
  MMP9-cleavable sequence
  MISC_FEATURE (3)..(3): X = S or T
  MISC_FEATURE (4)..(4): X = L or I
  MISC_FEATURE (5)..(5): X = S or T
PRXXX

SEQ ID NO: 204 - protein, 5 aa, Artificial sequence
  MMP9-cleavable sequence
LEATA

SEQ ID NO: 205 - protein, 10 aa, Artificial sequence
  MMP11-cleavable sequence
GGAANLVRGG

SEQ ID NO: 206 - protein, 10 aa, Artificial sequence
  MMP14-cleavable sequence
SGRIGFLRTA

SEQ ID NO: 207 - protein, 6 aa, Artificial sequence
  MMP-cleavable sequence
PLGLAG

SEQ ID NO: 208 - protein, 6 aa, Artificial sequence
  MMP-cleavable sequence
  misc_feature (6)..(6): Xaa can be any naturally occurring amino acid
PLGLAX

SEQ ID NO: 209 - protein, 6 aa, Artificial sequence
  MMP-cleavable sequence
  MOD_RES (4)..(4): METHYLATION
PLGCAG

SEQ ID NO: 210 - protein, 8 aa, Artificial sequence
  MMP-cleavable sequence
ESPAYYTA

SEQ ID NO: 211 - protein, 6 aa, Artificial sequence
  MMP-cleavable sequence
RLQLKL

SEQ ID NO: 212 - protein, 7 aa, Artificial sequence
  MMP-cleavable sequence
RLQLKAC

SEQ ID NO: 213 - protein, 7 aa, Artificial sequence
  MMP-, MMP9-, MMP14-cleavable sequence
  MISC_FEATURE (3)..(3): X = Citrulline
  MISC_FEATURE (5)..(5): X = Homophenylalanine
EPXGXYL

SEQ ID NO: 214 - protein, 5 aa, Artificial sequence
  Urokinase plasminogen activator (uPA)-cleavable sequence
SGRSA

SEQ ID NO: 215 - protein, 4 aa, Artificial sequence
  Urokinase plasminogen activator (uPA)-cleavable sequence
DAFK

SEQ ID NO: 216 - protein, 5 aa, Artificial sequence
  Urokinase plasminogen activator (uPA)-cleavable sequence
GGGRR

SEQ ID NO: 217 - protein, 4 aa, Artificial sequence
  Lysomal Enzyme-cleavable sequence
GFLG

SEQ ID NO: 218 - protein, 4 aa, Artificial sequence
  Lysomal Enzyme-cleavable sequence
ALAL

SEQ ID NO: 219 - protein, 2 aa, Artificial sequence
  Lysomal Enzyme-cleavable sequence
FK

SEQ ID NO: 220 - protein, 3 aa, Artificial sequence
  Cathepsin B-cleavable sequence
NLL

SEQ ID NO: 221 - protein, 6 aa, Artificial sequence
  Cathepsin D-cleavable sequence
  MISC_FEATURE (4)..(4): X = Ethionine
PICXFF

SEQ ID NO: 222 - protein, 8 aa, Artificial sequence
  Cathepsin K-cleavable sequence
GGPRGLPG

SEQ ID NO: 223 - protein, 6 aa, Artificial sequence
  Prostate Specific Antigen-cleavable sequence
HSSKLQ

SEQ ID NO: 224 - protein, 7 aa, Artificial sequence
  Prostate Specific Antigen
HSSKLQL

SEQ ID NO: 225 - protein, 9 aa, Artificial sequence
  Prostate Specific Antigen-cleavable sequence
HSSKLQEDA

SEQ ID NO: 226 - protein, 10 aa, Artificial sequence
  Herpes Simplex Virus Protease-cleavable sequence
LVLASSSFGY

SEQ ID NO: 227 - protein, 10 aa, Artificial sequence
  HIV Protease-cleavable sequence
GVSQNYPIVG

SEQ ID NO: 228 - protein, 10 aa, Artificial sequence
  CMV Protease-cleavable sequence
GVVQASCRLA

SEQ ID NO: 229 - protein, 4 aa, Artificial sequence
  Thrombin-cleavable sequence
  MISC_FEATURE (2)..(2): X = Piperidine
FXRS

SEQ ID NO: 230 - protein, 6 aa, Artificial sequence
  Thrombin-cleavable sequence
DPRSFL

SEQ ID NO: 231 - protein, 6 aa, Artificial sequence
  Thrombin-cleavable sequence
PPRSFL

SEQ ID NO: 232 - protein, 4 aa, Artificial sequence
  Caspase-3-cleavable sequence
DEVD

SEQ ID NO: 233 - protein, 5 aa, Artificial sequence
  Caspase-3-cleavable sequence
DEVDP

SEQ ID NO: 234 - protein, 8 aa, Artificial sequence
  Caspase-3-cleavable sequence
KGSGDVEG

SEQ ID NO: 235 - protein, 6 aa, Artificial sequence
  Interleukin 1beta converting enzyme-cleavable sequence
GWEHDG

SEQ ID NO: 236 - protein, 7 aa, Artificial sequence
  Enterokinase-cleavable sequence
EDDDDKA

SEQ ID NO: 237 - protein, 9 aa, Artificial sequence
  FAP-cleavable sequence
KQEQNPGST

SEQ ID NO: 238 - protein, 6 aa, Artificial sequence
  Kallikrein 2-cleavable sequence
GKAFRR

SEQ ID NO: 239 - protein, 4 aa, Artificial sequence
  Plasmin-cleavable sequence
DAFK

SEQ ID NO: 240 - protein, 4 aa, Artificial sequence
  Plasmin-cleavable sequence
DVLK

SEQ ID NO: 241 - protein, 4 aa, Artificial sequence
  Plasmin-cleavable sequence
DAFK

SEQ ID NO: 242 - protein, 7 aa, Artificial sequence
  TOP-cleavable sequence
ALLLALL

SEQ ID NO: 243 - protein, 10 aa, Artificial sequence
  Gly-Ser rich linker polypeptide sequence
SGGGGSGGGG

SEQ ID NO: 244 - protein, 10 aa, Artificial sequence
  Gly-Ser rich linker polypeptide sequence
GGGGSGGGGS

SEQ ID NO: 245 - protein, 6 aa, Artificial sequence
  Gly-Ser rich linker polypeptide sequence
GSGGGG

SEQ ID NO: 246 - protein, 6 aa, Artificial Sequence
  Hexa-His tag
HHHHHH

SEQ ID NO: 247 - protein, 248 aa, Homo sapiens
DIVMTQSPDSLAVSLGERATINCKSSQSVLYSNNNKNYLAWYQQKPGQPPKLLIYGASTRESWVPDRFSGSGSGTDFTLTISSLQAEDVAVYYCQQWYYYPYTFGQGTKVEIKGGGGSGGGGSGGGSGGGGSEVQLLESGGGLVQPGGSLRLSCAASGFTFSSYYMSWVRQAPGKGLEWVSDISGRGGQTNYADSVKGRFTISRDNSKNTLYLQMNSLRAEDTAVYYCARGGGSFANWGRGTLVTVSS

SEQ ID NO: 248 - protein, 113 aa, Homo sapiens
DIVMTQSPDSLAVSLGERATINCKSSQSVLYSNNNKNYLAWYQQKPGQPPKLLIYGASTRESWVPDRFSGSGSGTDFTLTISSLQAEDVAVYYCQQWYYYPYTFGQGTKVEIK

SEQ ID NO: 249 - protein, 116 aa, Homo sapiens
EVQLLESGGGLVQPGGSLRLSCAASGFTFSSYYMSWVRQAPGKGLEWVSDISGRGGQTNYADSVKGRFTISRDNSKNTLYLQMNSLRAEDTAVYYCARGGGSFANWGRGTLVTVSS

SEQ ID NO: 250 - protein, 17 aa, Artificial Sequence
  scFv183 VL HVR1
KSSQSVLYSNNNKNYLA

SEQ ID NO: 251 - protein, 7 aa, Artificial Sequence
  scFv183 VL HVR2
GASTRES

SEQ ID NO: 252 - protein, 9 aa, Artificial Sequence
  scFv183 VL HVR3
QQWYYYPYT

SEQ ID NO: 253 - protein, 6 aa, Artificial Sequence
  scFv183 VH HVR1
SSYYMS

SEQ ID NO: 254 - protein, 17 aa, Artificial Sequence
  scFv183 VH HVR2
DISGRGGQTNYADSVKG

SEQ ID NO: 255 - protein, 8 aa, Artificial Sequence
  scFv183 VH HVR3
RGGGSFAN

SEQ ID NO: 256 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 257 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPG